(19) United States
(12) Patent Application Publication
(10) Pub. No.: US 2018/0273538 A1
(43) Pub. Date: Sep. 27, 2018

(54) PYRROLO[2,3-d]PYRIMIDINE DERIVATIVES, A PROCESS FOR THEIR PREPARATION AND PHARMACEUTICAL COMPOSITIONS CONTAINING THEM

(71) Applicants: LES LABORATOIRES SERVIER, SURESNES CEDEX (FR); VERNALIS (R&D) LTD, Winnersh (GB)

(72) Inventors: Andrea FIUMANA, Ely (GB); Nicolas FOLOPPE, Cambridge (GB); Stuart RAY, Milton (GB); David WALMSLEY, Ely (GB); András KOTSCHY, Törökbálint (HU); Michael BURBRIDGE, Verneuil Sur Seine (FR); Francisco Humberto CRUZALEGUI, Brax (FR)

(21) Appl. No.: 15/763,626

(22) PCT Filed: Sep. 30, 2016
(86) PCT No.: PCT/EP2016/073403

§ 371 Date: Mar. 27, 2018

(30) Foreign Application Priority Data
Sep. 30, 2015 (FR) 15/59259

(51) Int. Cl. C07D 487/04; A61P 35/00; A61K 31/519; A61K 45/06; A61K 31/5377; C07D 519/00; C07D 471/04; A61K 31/437; A61K 31/444

(52) U.S. Cl. CPC C07D 487/04; A61P 35/00; A61K 31/519; A61K 45/06; A61K 31/444; C07D 519/00; C07D 471/04; A61K 31/437; A61K 31/5377

(57) ABSTRACT

Compounds of formula (I):

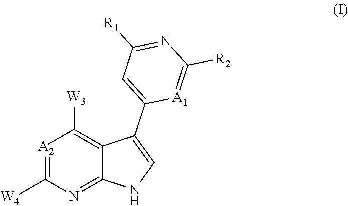

wherein R₁, R₂, W₃, W₄, A₁, and A₂ are as defined in the description.

Medicinal products containing the same which are useful in treating cancer, neurodegenerative disorders and metabolic disorders.

The present invention relates to new pyrrolo[2,3-d]pyrimidine derivatives, to a process for their preparation and to pharmaceutical compositions containing them.

The compounds of the present invention are new and have very valuable pharmacological characteristics in the field of oncology.

The present invention relates to the use of dual DYRK1/CLK1 inhibitors in the treatment of cancer, neurodegenerative disorders and metabolic disorders.

In cancer, the dual-specificity tyrosine-phosphorylation-regulated kinases DYRK1A and DYRK1B have been demonstrated to control several pathways that enhance cancer cell proliferation, migration and metastasis, induce resistance to cell death and repress responses to conventional and targeted anti-cancer therapies [Abbassi et al, Pharmacol Ther. 2015; 151:87-98; Ionescu et al Mini Rev Med Chem. 2012; 12(13): 1315-29; Friedman et al, J Cell Biochem. 2007; 102(2):274-9: Yoshida et al, Biochem Pharmacol. 2008; 76(11):1389-94]. Reported substrates of DYRK1A that are involved in this regulation of cancer progression and resistance to therapy include the transcription factors GLI1, STAT3 and FOXO1 [Mao et al, J Biol Chem. 2002; 277(38):35156-61; Matsuo et al, J Immunol Methods 2001; 247:141-51; Woods et al, Biochem J. 2001; 355(Pt 3):597-607]. DYRK1A is also believed to stabilise cancer-associated tyrosine kinase receptors such as EGFR and FGFR via interaction with the protein Sprouty2 [Ferron et al Cell Stem Cell. 2010:7(3):367-79; Aranda et al Mol Cell Biol. 2008; 28(19):5899-911]. DYRK1A, and also DYRK1B have been shown to be required for the induction of cell quiescence in response to treatment of cancer cells by chemotherapeutic agents and targeted therapies. This is important since it is known that quiescent cancer cells are relatively insensitive to most anti-cancer drugs and radiation [Ewton et al, Mol Cancer Ther. 2011; 10(11):2104-14; Jin et al, J Biol Chem. 2009; 284(34):22916-25]. For example, DYRK1A activates the DREAM multisubunit protein complex, which maintains cells in quiescence and protects against apoptosis [Litovchick et al, Genes Dev. 2011; 25(8):801-13]. DYRK1B has been demonstrated to prevent cell-cycle exit in response to chemotherapy via phosphorylation of Cyclin D1 [Zou et al, J Biol Chem. 2004; 279(26):27790-8]. DYRK1B has also been shown to protect against chemotherapy through a reduction in reactive oxygen species content [Hu et al, Genes Cancer. 2010; 1(8):803-811].

It is thus clear that the use of DYRK1A /DYRK1B inhibitors would constitute a novel anti-cancer treatment in a wide variety of cancers when used either alone or in combination with conventional therapy, radiation or targeted therapies as a strategy to combat resistance.

The role of DYRK1A in neurological disorders is well established. DYRK1A is associated with neurodegenerative disorders such as Alzheimer's, Parkinson's and Huntington's diseases, as well as with Down's syndrome, mental retardation and motor defects and [Abbassi et al, Pharmacol Ther. 2015; 151:87-98; Beker et al, CNS Neurol Disord Drug Targets. 2014; 13(1):26-33; Dierssen, Nat Rev Neurosci. 2012 December; 13(12):844-58]. DYRK1A has been identified as a major kinase phosphorylating the microtubule-associated protein TAU, leading to the formation of neurotoxic neurofibrillary tangles and neurodegeneration as seen in Alzheimer's [Azorsa et al, BMC Genomics. 2010; 11:25]. DYRK1A also alters the splicing of TAU pre-mRNA leading to an imbalance between TAU isoforms which is sufficient to cause neurodegeneration and dementia [Liu et al, Mol Neurodegener. 2008; 3:8]. It is not surprising, therefore, that DYRK1A is believed to be causally involved in the development of Alzheimer-like neurodegenerative diseases in Down Syndrome patients, where three copies of the DYRK1A gene are present on chromosome 21. In these individuals, increased DYRK1A activity also causes premature neuronal differentiation and a decrease in mature neurones [Hämmerle et al, Development. 2011; 138(12):2543-54].

It is thus clear that the use of DYRK1A inhibitors would offer a novel therapeutic approach for the treatment of neurodegenerative disorders, in particular Alzheimer's disease, as well as for other neurological conditions such as Down's syndrome. The CDC2-like kinase (CLK) family contains four isoforms (CLK1-4) which are important in regulating the function of the spliceosome complex [Fedorov et al, Chem Biol. 2011; 18(1):67-76]. This complex, comprised of small nuclear RNAs (snRNA) and a large number of associated proteins, regulates the splicing of pre-mRNAs to give mature protein-encoding mRNAs. CLK1 is known to regulate the activity of the spliceosome via phosphorylation of the constituent serine-arginine-rich (SR) proteins [Bullock et al, Structure. 2009; 17(3):352-62]. By controlling the activity of the spliceosome in this way, many genes are able express more than one mRNA leading to diversity in the translated proteins. The alternative protein isoforms transcribed from the same gene will often have different activities and physiological functions. Deregulation of alternative splicing has been linked to cancer, where a number of cancer-related proteins are known to be alternatively spliced [Druillennec et al, J Nucleic Acids. 2012; 2012:639062]. An example of an alternatively spliced protein in cancer is Cyclin D1, important for the progression of cancer cells through the cell cycle [Wang et al, Cancer Res. 2008; 68(14):5628-38]. It is thus clear that the use of CLK1 inhibitors would constitute a novel anti-cancer treatment in a wide variety of cancers when used either alone or in combination with conventional therapy, radiation or targeted therapies.

Alternative splicing regulated by CLK1 has also been described to play a role in neurodegenerative diseases, including Alzheimer's and Parkinson's, via phosphorylation of the SR proteins of the spliceosome [Jain et al, Curr Drug Targets. 2014; 15(5):539-50]. In the case of Alzheimer's, CLK1 is known to regulate the alternative splicing of the microtubule-associated protein TAU leading to an imbalance between TAU isoforms which is sufficient to cause neurodegeneration and dementia [Liu et al, Mol Neurodegener. 2008, 3:8].

It is thus clear that the use of CLK1 inhibitors would offer a novel therapeutic approach for the treatment of neurodegenerative disorders, in particular Alzheimer's disease, as well as for other neurological conditions such as Parkinson's.

In the treatment of both cancer and neurological disease, there is thus undoubtedly an urgent need for compounds which potently inhibit the DYRK1 and CLK1 kinases whilst not affecting other closely-related kinases. The DYRK1 and CLK1 kinases are members of the CMGC group, which includes the CDK and the GSK kinases, the chronic inhibition of which is believed to be a cause of toxicity to the patient. For example, common toxicities observed in the clinic with CDK inhibition are similar to those observed with conventional cytotoxic therapy, and include hematologic toxicity (leukopenia and thrombocytopenia), gastrointestinal toxicity (nausea and diarrhea), and fatigue [Kumar et al, Blood. 2015; 125(3):443-8]. The present invention describes a new class of DYRK1/CLK1 inhibitors which are highly selective for DYRK1 and CLK1 over these other kinases and which would thus be suitable for use in the treatment of these pathologies.

Diabetes type 1 and type 2 both involve deficiency of functional pancreatic insulin-producing beta cells. Restoring functional beta-cell mass is thus an important therapeutic goal for these diseases which affect 380 million people worldwide. Recent studies have shown that DYRK1A inhibition promotes human beta-cell proliferation in vitro and in vivo and, following prolonged treatment, can increase glucose-dependent insulin secretion [Dirice et al, Diabetes. 2016: 65(6): 1660-71; Wang et al, Nat Med. 2015; 21(4):383-8]. These observations clearly suggest that the use of potent and selective DYRK1A inhibitors would offer a novel therapeutic approach for the treatment and/or prevention of metabolic disorder's including diabetes and obesity.

The present invention relates more especially to compounds of formula (I):

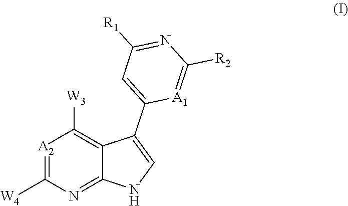

wherein:
R₁ and R₂ , each independently of the other, represent a hydrogen atom, a halogen atom, —NR₅R₅′ or a linear or branched (C₁-C₆)alkyl group,
W₃ represents a linear or branched (C₁-C₆)alkoxy, —O—(C₁-C₆)alkylene-Cy₁, —O—(C₀-C₆)-Cy₁-Cy₂, —NRₐR_{b}, —NRₐ—(C₀-C₆)alkylene-Cy₁, —NRₐ(C₀-C₆)alkylene-Cy₁-Cy₂, —NRₐ—(C₀-C₆)alkylene-Cy₁ -O—(C₁-C₆)alkylene-Cy₂, -Cy₁, -Cy₁-(C₀-C₆)alkylene-Cy₂, -Cy₁ -O—(C₀-C₆)alkylene-Cy₂, —(C₁-C₆)alkylene-Cy₁, —(C₂-C₆)alkenylene-Cy₁, —(C₂-C₆)alkynylene-Cy₁, —(C₁-C₆)alkylene-O—Cy₁, it being understood that the alkylene moieties defined hereinbefore may be linear or branched,
W₄ represents a cyano group, a cycloalkyl group, a linear or branched (C₁-C₆)alkyl group, a linear or branched (C₂-C₆)alkenyl group, a linear or branched (C₂-C₆)alkynyl group optionally substituted by a cycloalkyl group,
R₅ and R₅′ each independently of the others, represent a hydrogen atom or a linear or branched (C₁-C₆)alkyl group,
Rₐ and R_{b}, each independently of the other, represent a hydrogen atom or a linear or branched (C₁-C₆)alkyl group,
A₁ and A₂, each independently of the other, represent CH or a nitrogen atom,
Cy₁, Cy₂and Cy₃, independently of one another, represent a cycloalkyl group, a heterocycloalkyl group, an aryl or an heteroaryl group,
   wherein:
“aryl” means a phenyl, naphthyl, biphenyl or indenyl group,
“heteroaryl” means any mono- or bi-cyclic group composed of from 5 to 10 ring members, having at least one aromatic moiety and containing from 1 to 4 hetero atoms selected from oxygen, sulphur and nitrogen.
“cycloalkyl” means any mono- or bi-cyclic, non-aromatic, carbocyclic group containing from 3 to 11 ring members, which may include fused, bridged or spiro ring systems,
“heterocycloalkyl” means any mono- or bi-cyclic, non-aromatic, condensed or spiro group composed of from 3 to 10 ring members and containing from 1 to 3 hetero atoms or groups selected from oxygen, sulphur, SO, SO₂ and nitrogen, which may include fused, bridged or spiro ring systems,
“—(C₀-C₆)alkylene-” refers either to a covalent bond (—C₀alkylene-) or to an alkylene group containing 1, 2, 3, 4, 5 or 6 carbon atoms,
it being possible for the aryl, heteroaryl, cycloalkyl and heterocycloalkyl groups so defined and the alkyl, alkenyl, alkynyl, alkylene, alkenylene, alkynylene to be substituted by from 1 to 4 groups selected from linear or branched (C₁-C₆)alkyl, linear or branched (C₂-C₆)alkenyl group, linear or branched (C₂-C₆)alkynyl group, linear or branched (C₁-C₆)alkoxy optionally substituted by —NR_{c}R_{d} or by from 1 to 3 halogen atoms, linear or branched (C₁-C₆)alkyl-S—, hydroxy, oxo (or N-oxide where appropriate), nitro, cyano, —C(O)—OR_{c}, —C(O)—R_{c}, —O—C(O)—R_{d}, —C(O)—NR_{c}R_{d}, —NR_{c}—C(O)—R_{d}, —NR_{c}R_{d}, linear or branched (C₁-C₆)polyhaloalkyl, or halogen, it being understood that R_{c} and R_{d} independently of one another represent a hydrogen atom or a linear or branched (C₁-C₆)alkyl group,
to their enantiomers and diastereoisomers, and to addition salts thereof with a pharmaceutically acceptable acid or base.

Among the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid etc.

Among the pharmaceutically acceptable bases there may be mentioned, without implying any limitation, sodium hydroxide, potassium hydroxide, triethylamine, tert-butylamine etc.

Advantageously, R₁ represents a hydrogen and R₂ a —NH₂ group.

In one embodiment of the invention, A₁ represents a CH group.

In another embodiment of the invention, A₁ represents a nitrogen atom.

In a preferred embodiment of the invention, A₂ represents a nitrogen atom.

Alternatively, A₂ represents a CH group. When A₂ represents a CH group, A₁ represents preferably a CH group.

In another embodiment of the invention, W₃ represents a linear or branched (C₁-C₆)alkoxy, —O—(C₀-C₆)alkylene-Cy₁, —O—(C₀-C₆)alkylene-Cy₁-Cy₂, —NRₐ—(C₁-C₆)alkylene-Cy₁-Cy₂, —NRₐ—(C₀-C₆)alkylene-Cy₁—O—(C₁-C₆)alkylene-Cy₂, -Cy₁—O—(C₁-C₆)alkylene-Cy₂, -(C₁-C₆)alkylene-Cy₁, —(C₂-C₆)alkenylene-Cy₁, —(C₂-C₆)alkynylene-Cy₁, —(C₁-C₆)alkylene-O-Cy₁, it being understood that the alkylene moieties defined hereinbefore may be linear or branched.

Alternatively, W₃ represents a Cy₁ group selected from: 1,3-benzodioxolyl, 1H-indolyl, phenyl, pyridinyl, 2,3-dihydro-1,4-benzodioxinyl, 1-benzothiophenyl, 1-benzofuranyl, 3,4-dihydronaphthalenyl, 1,2,3,4-tetrahydronaphthalenyl, 3,4-dihydro-2H-1,4-benzoxazinyl, wherein the preceding groups are optionally substituted according to the definition mentioned previously.

In an other embodiment, W₃ represents: (i) a —NRₐ-Cy₁ group, wherein Cy₁ represents a group selected from: phenyl, 2,3-dihydro-1H-indene and 1,2,3,4-tetrahydronaphthalene, wherein the preceding groups are optionally substituted according to the definition mentioned previously; or (ii) a —NRₐ—(C₁-C₆)alkylene-Cy₁ group, wherein Cy₁ represents a group selected from: phenyl, pyridinyl, furanyl, thiophenyl, 1H-pyrazolyl, 1,3-thiazolyl, 1,2-oxazolyl, cyclohexyl, cyclopropyl and 1H-indolyl, wherein the preceding groups are optionally substituted according to the definition mentioned previously.

In a specific embodiment, W₃ represents a -phenylene-(C₀-C₆)alkylene-Cy₂.

More preferably, W₃ represents —O—(C₁-C₆)alkylene-Cy₁ or —NRₐ—(C₁-C₆)alkylene-Cy₁, wherein Cy₁ is a phenyl or a pyridinyl group, these latter group being optionally substituted by one or two groups selected from methoxy, methyl or halogen.

Preferred W₄ groups are as follows: methyl; propan-2-yl; prop-1-en-2-yl; ethenyl; cyano; ethynyl; cyclopropyl; cyclopropylethynyl. Methyl group is even more preferred.

Preferred compounds according to the invention are included in the following group:
5-(2-aminopyridin-4-yl)-N-(2-methoxybenzyl)-2-methyl-7H-pyrrolo[2,3-d]-pyrimidin-4-amine,
4-[2-methyl-4-(thiophen-3-ylmethoxy)-7H-pyrrolo[2,3-d]pyrimidin-5-yl]pyridin-2-amine,
5-(2-aminopyridin-4-yl)-N-(2,6-dichlorobenzyl)-2-methyl-7H-pyrrolo[2,3-d]pyrimidin-4-amine,
5-(2-aminopyridin-4-yl)-N-(2,6-difluorobenzyl)-2-methyl-7H-pyrrolo[2,3-d]pyrimidin-4-amine,
5-(2-aminopyridin-4-yl)-2-methyl-N-(2-methylbenzyl)-7H-pyrrolo[2,3-d]pyrimidin-4-amine,
5-(2-aminopyridin-4-yl)-N-(2-chloro-6-fluorobenzyl)-2-methyl-7H-pyrrolo[2,3-d]pyrimidin-4-amine,
5-(2-aminopyridin-4-yl)-2-methyl-N-[(3-methylpyridin-2-yl)methyl]-7H-pyrrolo[2,3-d]pyrimidin-4-amine,
5-(2-aminopyridin-4-yl)-N-[(3-fluoropyridin-2-yl)methyl]-2-methyl-7H-pyrrolo[2,3-d]pyrimidin-4-amine,
5-(2-aminopyridin-4-yl)-N-(2,6-difluorobenzyl)-2-methyl-7H-pyrrolo[2,3-d]pyrimidin-4-amine,
   their enantiomers and diastereoisomers, and addition salts thereof with a pharmaceutically acceptable acid or base.
   The invention relates also to a process for the preparation of compounds of formula (I), which process is characterised in that there is used as starting material the compound of formula (II):

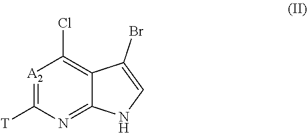

wherein T represents a halogen atom, a methane-sulfanyl group, a cycloalkyl group or a linear or branched (C₁-C₆)alkyl group, and A₂ is as defined in formula (I), which, compound is subjected to a nucleophilic substitution in the presence of an appropriate alcohol or amine derivative, or subjected to coupling with an appropriate boronic acid derivative,
to yield the compound of formula (III):

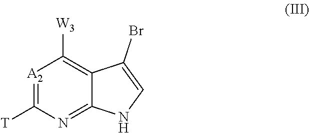

wherein T is as defined previously, A₂ and W₃ are as defined in formula (I),
which compound of formula (III) is either:
   (i) converted into its methanesulfonyl derivative when T represents a methanesulfanyl group, then reacted with NaCN and further subjected to coupling with an appropriate boronic acid derivative,
   (ii) or directly subjected to coupling with an appropriate boronic acid derivative,
   (iii) or subjected to coupling with 4,4,4′,4′,5,5,5′, 5′-octamethyl-2,2′,-bi-1,3,2-dioxaborolane to yield:

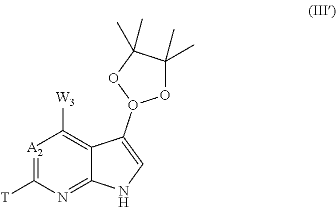

which compound of formula (III′) is further reacted with the appropriate halide,
   to yield compound of formula (IV):

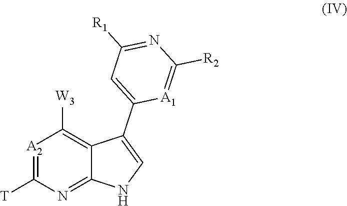

wherein T′ represents represents a halogen atom, a cyano group, a cycloalkyl group or a linear or branched (C₁-C₆)alkyl group, and A₁, A₂, R₁, R₂ and W₃ are as defined is formula (I),
which compound of formula (IV):
may be subjected to coupling with an appropriate alkynyl (or alkenyl) boronic acid derivative or alkynyl (or alkenyl) (trifluoro)borate derivative salt, when T′ represents a halogen atom,
to yield the compounds of formula (I),
which compound of formula (I) may be purified according to a conventional separation technique, which is converted, if desired, into its addition salts with a pharmaceutically acceptable acid or base and which is optionally separated into its isomers according to a conventional separation technique,
it being understood that, at any time considered appropriate in the course of the above-described process, certain groups (hydroxy, amino, . . . ) of the reagents or intermediates of synthesis may be protected and then deprotected according to the requirements of synthesis.

The invention relates also to an alternative process for the preparation of compounds of formula (I), which process is characterised in that there is used as stalling material the compound of formula (II):

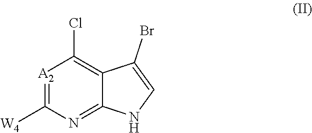

wherein W₄ and A₂ are as defined in formula (I),
which compound of formula (II) is subjected to coupling with an appropriate boronic acid derivative,
to yield compound of formula (V):

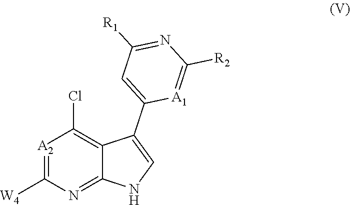

wherein A₁, A₂, R₁, R₂ , and W₄ are as defined in formula (I),
which compound of formula (V) is either subjected to a nucleophilic substitution, or subjected to a coupling reaction with an appropriate boronic acid derivative, or subjected to a coupling with a compound or formula

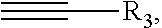

wherein R₃ represents a hydrogen or Cy₁,
to yield the compounds of formula (I),
which compound of formula (I) may be purified according to a conventional separation technique, which is converted, if desired, into its addition salts with a pharmaceutically acceptable acid or base and which is optionally separated into its isomers according to a conventional separation technique,
it being understood that, at any time considered appropriate in the course of the above-described process, certain groups (hydroxy, amino . . . ) of the reagents or intermediates of synthesis may be protected and then deprotected according to the requirements of synthesis.

The compound of formula (II), the alcohol and amino derivatives the boronic acid derivatives, the borate salt derivatives and

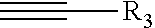

mentioned above are either commercially available or can be obtained by the person skilled in the art using conventional chemical reactions described in the literature.

Pharmacological study of the compounds of the invention has shown that they are powerful DYRK1/CLK1 inhibitors which are highly selective for DYRK1 and CLK1 over other kinases such as CDK9.

More especially, the compounds according to the invention will be useful in the treatment of chemo- or radio-resistant cancers.

Among the cancer treatments envisaged there may be mentioned, without implying any limitation, haematological cancer (lymphoma and leukemia) and solid tumors including carcinoma, sarcoma, or blastoma. There may be mentioned more preferably acute megakaryoblastic leukaemia (AMKL), acute lymphoblastic leukaemia (ALL), ovarian cancer, pancreatic cancer, gastrointestinal stromal tumours (GIST), osteosarcoma (OS), colorectal carcinoma (CRC), neuroblastoma and glioblastoma.

In another embodiment, the compounds of the invention will useful in the treatment of neurodegenerative disorders such as Alzheimer's, Parkinson's and Huntington's diseases, as well as with Down's syndrome, mental retardation and motor defects.

Alternatively, the compounds of the invention could be used in the treatment and/or prevention of metabolic disorders including diabetes and obesity.

The present invention relates also to pharmaceutical compositions comprising at least one compound of formula (I) in combination with one or more pharmaceutically acceptable excipients.

Among the pharmaceutical compositions according to the invention there may be mentioned more especially those that are suitable for oral, parenteral, nasal, per- or trans-cutaneous, rectal, perlingual, ocular or respiratory administration, especially tablets or dragées, sublingual tablets, sachets, paquets, capsules, glossettes, lozenges, suppositories, creams, ointments, dermal gels, and drinkable or injectable ampoules.

The dosage varies according to the sex, age and weight of the patient, the administration

route, the nature of the therapeutic indication, or of any associated treatments, and ranges from 0.01 mg to 5 g per 24 hours in one or more administrations.

Furthermore, the present invention relates also to the combination of a compound of formula (I) with an anticancer agent selected from genotoxic agents, mitotic poisons, anti-metabolites, proteasome inhibitors, kinase inhibitors, signaling pathway inhibitors, phosphatase inhibitors, apoptosis inducers and antibodies, and also to pharmaceutical compositions comprising that type of association and then use in the manufacture of medicaments for use in the treatment of cancer.

The combination of a compound of formula (I) with an anticancer agent may be administered simultaneously or sequentially. The administration route is preferably the oral route, and the corresponding pharmaceutical compositions may allow the instantaneous or delayed release of the active ingredients. The compounds of the combination may moreover be administered in the form of two separate pharmaceutical compositions, each containing one of the active ingredients, or in the form of a single pharmaceutical composition, in which the active ingredients are in admixture.

The compounds of the invention may also be used in association with radiotherapy in the treatment of cancer.

### List of Abbreviations

| Abbreviation | Name |
|---|---|
| Ac | acetyl |
| aq. | Aqueous |
| Bn | benzyl |
| Boc | tert-butyloxycarbonyl protecting group |
| dppf | 1,1′-bis(diphenylphosphino)ferrocene |
| DCM | dichloromethane |
| DEAD | diethyl azodicarboxylate |
| DIBAL | diisobutylaluminium hydride |
| DMAP | 4-diméthylaminopyridine |
| DMF | N,N-dimethylformamide |
| DMSO | dimethyl sulfoxide |
| dtbpf | 1,1′-bis(di-tert-butylphosphino)ferrocene |
| eq. | equivalent |
| Et | ethyl |
| IPA | isopropanol |
| HPLC-MS | liquid chromatography-mass spectrometry |
| LiHMDS | lithium bis(trimethylsilyl)amide |
| mCBPA | meta-chloroperoxybenzoic acid |
| Me | methyl |
| NBS | N-bromosuccinimide |
| ⁿBu | n-butyl |
| ⁿBuPAd₂ | n-butyldiademantylphosphine |
| Pd/C | palladium on carbon |
| Ph | phenyl |
| PPh₃ | triphenylphosphine |
| pTSA | para-toluenesulfonic acid |
| RT | retention time |
| sat. | saturated |
| SEM | [2-(trimethylsilyl)ethoxy]methyl |
| ^{t}Bu | tert-butyl |
| TFA | trifuoroacetic acid |
| THF | tetrahydrofurane |

### General Procedures

All reagents obtained from commercial sources were used without further purification. Anhydrous solvents were obtained from commercial sources and used without further drying. Flash chromatography was performed with pre-packed silica gel cartridges (Strata SI-1; 61Å, Phenomenex, Cheshire UK or 1ST Flash II, 54Å, Argonaut, Hengoed, UK) or by automated flash chromatography using a Combiflash R_{f} apparatus (Teledyne Isco Inc.) using RediSep R_{f} prepacked silica columns (Teledyne Isco Inc.) or SilaSep pre-packed columns (Silicycle Inc.). Thin layer chromatography was conducted with 5×10 cm plates coated with Merck Type 60 F₂₅₄ silica gel.

The compounds of the present invention were characterized by high performance liquid chromatography-mass spectroscopy (HPLC-MS) on either an Agilent HP1200 Rapid Resolution Mass detector 6140 multimode source M/z range 150 to 1000 amu or an Agilent HP1100 Mass detector 1946D ESI source M/z range 150 to 1000 amu. The conditions and methods listed below are identical for both machines.
Column for 7.5 min run: GeminiNX, 5 μm, C18, 30×2.1 mm (Phenomenex) or Zorbax Eclipse Phis, 3.5 μm, C18, 30×2.1 mm (Agilent). Temperature: 35° C.
Column for 3.75 min run: GeminiNX, 5 μm, C18, 30×2.1 mm (Phenomenex) or Zorbax Eclipse Plus, 3.5 μm, C18, 30×2.1 mm (Agilent). Temperature: 35° C.
Column for 1.9 min run: Inetex, 2.5 μm, C18, 50×2.1 mm (Phenomenex) or Accucore, 2.6 μm, C18, 50×2.1 mm.
Temperature: 55° C.
Mobile Phase: A—H₂O+10 mmol/ammonium formate+0.08% (v/v) formic acid at pH ca 3.5.
B—95% Acetonitrile+5% A+0.08% (v/v) formic acid.
Injection Volume: 1 μL
Method A “Short” method gradient table, either positive (pos) or positive and negative (pos/neg) ionisation

| Time | Solvent A | Solvent B | Flow |
|---|---|---|---|
| (min) | (%) | (%) | (mL/min) |
| 0 | 95 | 5 | 1 |
| 0.25 | 95 | 5 | 1 |
| 2.50 | 5 | 95 | 1 |
| 2.55 | 5 | 95 | 1.7 |
| 3.60 | 5 | 95 | 1.7 |
| 3.65 | 5 | 95 | 1 |
| 3.70 | 95 | 5 | 1 |
| 3.75 | 95 | 5 | 1 |

Method B “Super Short” method gradient table, either positive (pos) or positive and negative (pos/neg) ionisation

| Time | Solvent A | Solvent B | Flow |
|---|---|---|---|
| (min) | (%) | (%) | (mL/min) |
| 0 | 95 | 5 | 1.3 |
| 0.12 | 95 | 5 | 1.3 |
| 1.30 | 5 | 95 | 1.3 |
| 1.35 | 5 | 95 | 1.6 |
| 1.85 | 5 | 95 | 1.6 |
| 1.90 | 5 | 95 | 1.3 |
| 1.95 | 95 | 5 | 1.3 |

Detection: UV detection at 230, 254 and 270 nm.

The compounds of the present invention were also characterized by Nuclear Magnetic Resonance (NMR). Analysis was performed with a Broker DPX-400 spectrometer and proton NMR spectra were measured at 400 MHz. The spectral reference was the known chemical shift of the solvent. Proton NMR data is reported as follows: chemical shift (δ) in ppm, followed by the multiplicity, where s=singlet, d=doublet, t=triplet, q=quartet, m=multiplet, dd=doublet of doublets, dt=doublet of triplets, dm=doublet of multiplets, ddd=doublet of double doublets, td=triplet of doublets, qd=quartet of doublets and br=broad, and finally the integration.

Some compounds of the invention were purified by preparative HPLC. These were performed on a Waters FractionLynx MS autopurification system, with a Gemini® 5 μm C18(2) 100 mm×20 mm i.d. column from Phenomenex, running at a flow rate of 20 cm³min⁻¹ with UV diode array detection (210-400 nm) and mass-directed collection At pH 4: solvent A=10 mM ammonium acetate in HPLC grade water+0.08% v/v formic acid. Solvent B=95% v/v HPLC grade acetonitrile+5% v/v solvent A+0.08% v/v formic acid.

At pH 9: solvent A=10 mM ammonium acetate in HPLC grade water+0.08% v/v ammonia solution. Solvent B=95% v/v HPLC grade acetonitrile+5% v/v solvent A+0.08% v/v ammonia solution.

The mass spectrometer was a Waters Micromass ZQ2000 spectrometer, operating in positive or negative ion electrospray ionisation modes, with a molecular weight scan range of 150 to 1000.

Some compounds of the present invention were characterised using an Agilent 1290 Infinity II series instrument connected to an Agilent TOF 6230 single quadrupole with an ESI source. High resolution mass spectra were recorded in positive-negative switching mode ionization unless otherwise stated. UV detection was by diode array detector at 230, 254 and 270 nm. Column: Thermo Accucore 2.6 μM C18, 50×2 mm, at 55° C. column temperature. Buffer A: Water/10 mM ammonium formate/0.04% (v/v) formic acid pH=3.5. Buffer B: Acetonitrile/5.3 % (v/v) A/0.04% (v/v) formic. (Injection volume: 1 μL).

The following Preparations and Examples illustrate the invention without limiting it in any way.

### General Procedure I

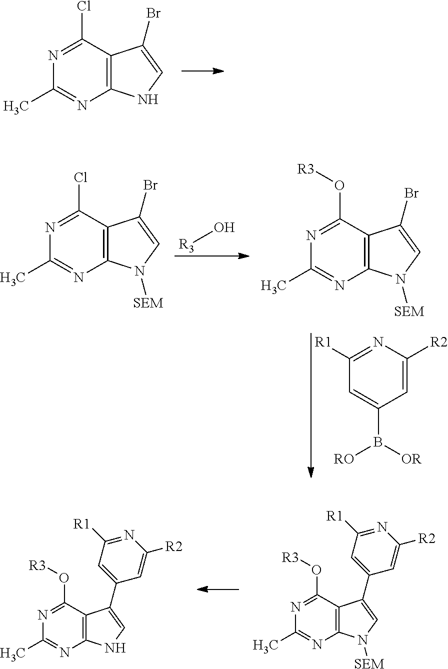

### General Procedure II

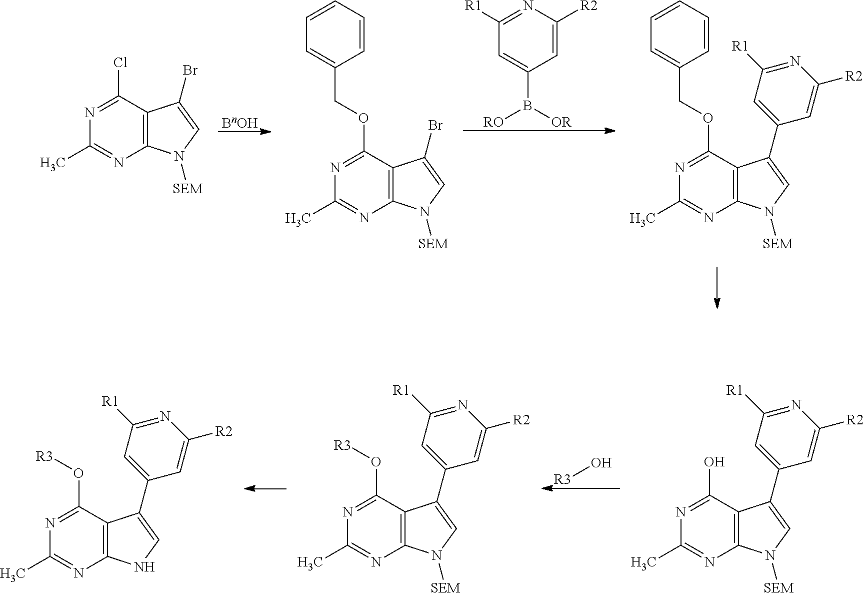

### General Procedure III

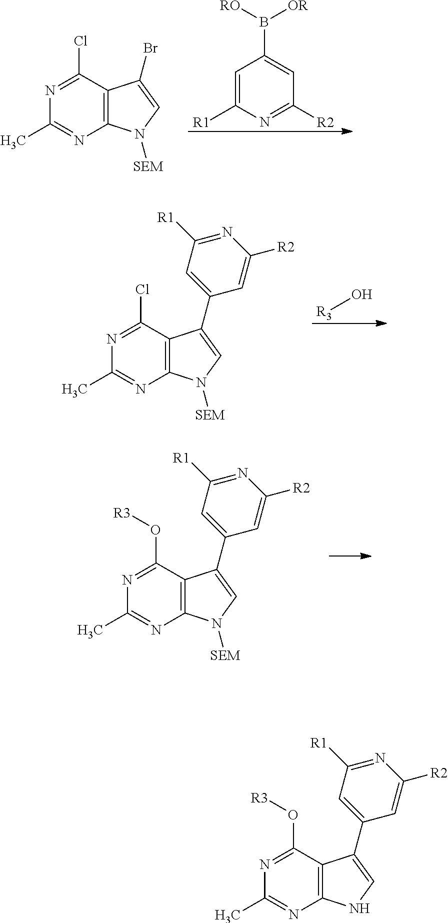

In General Procedures I, II and III:
R₁ and R₂ are as defined in formula (I),
R₃ represents a linear or branched (C₁-C₆)alkyl group, —(C₀-C₆)alkylene-Cy₁, —(C₀-C₆)alkylene-Cy₁-Cy₂, it being understood that Cy₁ and Cy₂, independently of one another, represent a cycloalkyl group, a heterocycloalkyl group, an aryl or an heteroaryl group.

### EXAMPLE 1

### 4-methoxy-2-methyl-5-(pyridin-4-yl)-7H-pyrrolo[2,3-d]pyrimidine

### Step 1: 5-bromo-4-chloro-2-methyl-7-{[2-(trimethylsilyl)ethoxy]methyl}-7H-pyrrolo[2,3-d]pyrimidine (Preparation 1)

To a solution of 5-bromo-4-chloro-2-methyl-7H-pyrrolo[2,3-d]pyrimidine (1 g, 4.06 mmol) in DMF (30 mL) was added NaH (60% in mineral oil, 1 eq) at 0° C. under N₂. The reaction mixture was stirred for 30 min before adding SEM-Cl (1.1 eq) at 0° C. and allowed to warm to room temperature overnight under N₂. The reaction mixture was diluted with diethyl ether (100 mL), washed with brine (4×50 mL), dried over MgSO₄ and concentrated in vacuo. The residue was purified via flash chromatography using EtOAc and isohexane as eluent to give the product (1.18 g, 3.13 mmol, 77%) as a white solid.

¹H NMR (399 MHz, DMSO-d6) δ 8.12 (s, 1H), 5.65 (s, 2H), 3.67-3.57 (m, 2H), 2.74 (s, 3H), 0.98-0.87 (m, 2H), 0.00 (s, 9H).

LC/MS (method B): RT=1.59 min; m/z=RT=1.59 min; m/z=377 [M+H]⁺

### Step 2: 5-bromo-4-methoxy-2-methyl-7-{[2-(trimethylsilyl)ethoxy]methyl}-7H-pyrrolo [2,3-d]pryimidine (Preparation 2)

To a suspension of NaH (60% in mineral oil, 2 eq) in THF (10 mL) was added MeOH (1.3 eq) dropwise at 0° C. under N₂. Stirred for 10 min before adding a solution of the compound obtained in Step 1 (0.5 g, 1.3 mmol) in THF (3 mL). The reaction mixture was stirred at 0° C. for 30 min and allowed to warm to room temperature over 1 hour. The reaction mixture was diluted with sat. aq. NH₄Cl solution (20 mL) and EtOAc (20 mL). The organic layer was separated, washed with brine, dried over MgSO₄ and concentrated in vacuo to give the product (0.494 g, 1.3 mmol, 100%) as a clear oil. The compound was used without further purification.

¹H NMR (399 MHz, DMSO-d6) δ 7.75 (s, 1H), 5.59 (s, 2H), 4.12 (s, 3H), 3.64-3.55 (m, 2H), 2.65 (s, 3H), 0.97-0.87 (m, 2H), 0.00 (s, 9H).

LC/MS (method B): RT=1.53 min; m/z=374 [M+H]⁺

### Step 3: 4-methoxy-2-methyl-5-(pyridin-4-yl)-7-{[2-(trimethylsilyl)ethoxy]methyl}-7H-pyrrolo[2,3-d]pyrimidine (Preparation 3)

The compound obtained in Step 2 and (pyridin-4-yl)boronic acid (1.5 eq) were dissolved in THF/water (6:1, 5 mL) under N₂. Potassium carbonate (3 eq) and Pd(dtbpf)Cl₂ (10% wt) were added and the resulting mixture was degassed under N₂ for 5 minutes. The reaction mixture was heated at 120° C. on a CEM microwave reactor for 1 hour. The reaction mixture was diluted with water (10 mL) and EtOAc (20 mL). The organic layer was separated, washed with brine, dried over MgSO₄ and concentrated in vacuo. The residue was purified via flash chromatography using EtOAc and isohexane as eluent to give the product (0.11 g, 0.30 mmol, 44%) as an oil.

¹H NMR (399 MHz, DMSO-d6) δ 8.67-8.61 (m, 2H), 8.11 (s, 1H), 7.83-7.77 (m, 2H), 5.68 (s, 2H), 4.13 (s, 3H), 3.70-3.61 (m, 2H), 2.68 (s, 3H), 0.99-0.88 (m, 2H), 0.00 (s, 9H).

LC/MS (method A): RT=1.37 mm; m/z=371 [M+H]⁺

### Step 4: 4-methoxy-2-methyl-5-(pyridin-4-yl)-7H-pyrrolo[2,3-d]pyrimidine (Preparation 4)

To a solution of the compound obtained in Step 3 (0.11 g, 0.3 mmol) in THF (3 mL) was added ethylenediamine (5 eq) followed by TBAF (1M solution in THF, 5 eq). The reaction was heated at 120° C. on a CEM microwave reactor for 1 hour. The reaction mixture was diluted with water (10 mL) and EtOAc (10 mL). The organic layer was separated, washed with brine, dried over MgSO₄ and concentrated in vacuo. The residue was then triturated with EtOAc to give the product (15 mg, 0.06 mmol, 21%) as a white solid.

¹H NMR (399 MHz, DMSO-d6) δ 12.33 (s, 1H), 8.58-8.50 (m, 2H), 7.85 (s, 1H), 7.78-7.72 (m, 2H), 4.05 (s, 3H), 2.57 (s, 3H).

LC/MS (method A): RT=1.49 mm; m/z=241 [M+H]⁺

### EXAMPLE 6

### 2-methyl-5-(2-methylpyridin-4-yl)-4-[(3R)-piperidin-3-ylmethoxy]-7H-pyrrolo[2,3-d]pyrimidine

### Step 1: 4-(benzyloxy)-5-bromo-2-methyl-7-{[2-(trimethylsilyl)ethoxy]methyl}-7H-pyrrolo[2,3-d]pyrimidine

Starting from 5-bromo-4-chloro-2-methyl-7-{[2-(trimethylsilyl)ethoxy]methyl}-7H pyrrolo[2,3-d]pyrimidine (Example 1, Step 1) (5 g, 13.27 mmol) and benzyl alcohol (1.3 eq) following procedure described in Preparation 2, the desired product (5.4 g, 12 mmol, 91%) was obtained as a light yellow oil.

¹H NMR (399 MHz, DMSO-d6) δ 7.77 (s, 1H), 7.68-7.60 (m, 2H), 7.54-7.45 (m, 2H), 7.47-7.38 (m, 1H), 5.67 (s, 2H), 5.60 (s, 2H), 3.65-3.55 (m, 2H), 2.67 (s, 3H), 0.97-0.87 (m, 2H), 0.00 (s, 9H).

LC/MS (method A): RT=3.04 min; m/z=450 [M+H]⁺

### Step 2: 4(benzyloxy)-2-methyl-5-(2-methylpyridin-4-yl)-7-{[2-(trimethylsilyl)ethoxy]methyl}-7H-pyrrolo[2,3-d]pyrimidine

Starting from the compound obtained in Step 1 (2 g, 4.46 mmol) and (2-methylpyridin-4-yl)boronic acid (1.2 eq) following procedure described in Preparation 3. The residue was purified via flash chromatography using EtOAc and isohexane as eluent to give the product (1.311 g, 2.8 mmol, 64%) as a brown oil.

¹H NMR (399 MHz, DMSO-d6) δ 8.36 (dd, 1H), 8.08 (s, 1H), 7.66-7.40 (m, 7H), 5.67 (s, 2H), 5.63 (s, 2H), 3.69-3.60 (m, 2H), 2.71 (s, 3H), 2.31 (s, 3H), 0.99-0.90 (m, 2H), 0.00 (s, 9H).

### Step 3: 2-methyl-5-(2-methylpyridin-4-yl)-7-{[2-(trimethylsilyl)ethoxy]methyl}-7H-pyrrolo[2,3-d]pyrimidin-4-ol (Preparation 5)

A suspension of the compound obtained in Step 2 (1.311 g, 2.8 mmol) and Pd/C (10% in wt) in EtOH (40 mL) was agitated under H₂ at room temperature for 2 h. The suspension was filtered through a plug of celite and concentrated in vacuo. the residue was triturated with isohexane to give the product (0.886 g, 2.39 mmol, 84%) as an off-white solid

¹H NMR (399 MHz, DMSO-d6) δ 12.14 (s, 1H), 8.47-8.40 (m, 1H), 8.01-7.91 (m, 3H), 5.54 (s, 2H), 3.62 (dd, 2H), 2.53 (s, 3H), 2.43 (s, 3H), 0.92 (dd, 2H), 0.00 (s, 9H).

### Step 4: tert-butyl (3R)-3-({[2-methyl-5-(2-methylpyridin-4-yl)-7-{[2-(trimethylsilyl) ethoxy]methyl}-7H-pyrrolo[2,3-d]pyrimidin-4-yl]oxy}methyl)piperidine-1-carboxylate (Preparation 6)

To a solution of the compound obtained in Step 3 (100 mg, 0.27 mmol) and tert-butyl (3R)-3-(hydroxymethyl)piperidine-1-carboxylate (1.5 eq) in THF (5 mL) was added PPh₃ (1.5 eq) at room temperature under N₂. The reaction mixture was allowed to stir at room temperature for 10 minutes and then cooled in an ice-bath before adding DEAD (1.5 eq). The ice-bath was removed and the reaction mixture allowed to stir for 2 hours at room temperature. The reaction mixture was concentrated in vacuo and the residue purified via flash chromatography using EtOAc and isohexane as eluent to give the product (122 mg, 0.214 mmol, 80%) as a clear oil.

¹H NMR (399 MHz, DMSO-d6) δ 8.51 (d, 1H), 8.08 (s, 1H), 7.72 (s, 1H), 7.61 (d, 1H), 5.67 (s, 2H), 4.51 (dd, 1H), 4.40 (dd, 1H), 3.68-3.59 (m, 2H), 3.43 (s, 9H), 2.66 (s, 3H), 2.56 (s, 3H), 1.88 (d, 1H), 1.69 (s, 1H), 1.47-1.19 (m, 7H), 0.99-0.87 (m, 2H), 0.00 (s, 9H).

### Step 5: 2-methyl-5-(2-methylpyridin-4-yl)-4-[(3R)-piperidin-3-ylmethoxy]-7H-pyrrolo[2,3-d]pyrimidin (Preparation 7)

To a solution of the compound obtained in Step 4 (78 mg, 0.137 mmol) in DCM (5 mL) was added TFA (3 mL) under N₂ at room temperature and stirred for 3 hours. The reaction mixture was loaded directly into a scx-2 column (10 g), washed with MeOH and DCM and eluted with 1N NH₃ solution in MeOH. The fractions were concentrated in vacuo and the residue was purified via flash chromatography using 2N NH₃ solution m MeOH and DCM as eluent to give the desired product (18 mg, 0.024 mmol, 17%) as a white solid.

¹H NMR (399 MHz, DMSO-d6) δ 12.24 (s, 1H), 8.38 (d, 1H), 7.80 (s, 1H), 7.66 (d, 1H), 7.54 (dd, 1H), 4.30 (qd, 2H), 3.05-2.96 (m, 1H), 2.84 (dt, 1H), 2.54 (s, 3H), 2.51 (s, 3H), 2.47-2.36 (m, 1H), 2.32 (dd, 1H), 1.97-1.86 (m, 1H), (m, 1H), 1.79 (dd, 1H), 1.62-1.49 (m, 1H), 1.46-1.02 (m, 3H).

LC/MS (method A): RT=1.35 mm; m/z=338 [M+H]⁺

### EXAMPLE 20

### 4-[2-methyl-4-(1-phenylethoxy)-7H-pyrrolo[2,3-d]pyrimidin-5-yl]pyridin-2-amine

### Step 1: 4-(4-chloro-2-methyl-7-{[2-(trimethylsilyl)ethoxy]methyl}-7H-pyrrolo[2,3-d]pyrimidin-5-yl)pyridin-2-amine

Starting from 5-bromo-4-chloro-2-methyl-7-{[2-(trimethylsilyl)ethoxy]methyl}-7H-pyrrolo[2,3-d]pyrimidin (0.91 g, 2.42 mmol) and 4-(tetramethyl-1,3,2-dioxaborolan-2-yl)pyridin-2-amine (1.1 eq) following procedure described in Preparation 3, the desired product (0.257 g, 0.659 mmol, 27%) was obtained as a white solid.

¹H NMR (399 MHz, DMSO-d6) δ 8.02 (t, 2H), 6.74-6.63 (m, 2H), 6.08 (s, 2H), 5.72 (s, 2H), 3.66 (dd, 2H), 2.76 (s, 3H), 0.99-0.88 (m, 2H), 0.00 (s, 9H).

LC/MS (method A): RT=2.16 min; m/z=390 [M+H]⁺

### Step 2: 4-[2-methyl-(1-phenylethoxy)-7-{[2-trimethylsilyl)ethoxy]methyl}-7H-pyrrolo[2,3-d]pyrimidin-5-yl]pyridin-2-amine

Starting from the compound obtained in Step 1 (100 mg, 0.25 mmol) and 1-phenylethan-1-ol (1.3 eq) following procedure described in Preparation 2, the product (107 mg, 0.224 mmol, 90%) was obtained as an oil.

LC/MS (method B): RT=1.38 min; m/z=476 [M+H]⁺

### Step 3: 4-[2-methyl-4-(1-phenylethoxy)-7H-pyrrolo[2,3-d]pyrimidin-5-yl]pyridin-2-amine

Starting from the compound obtained in Step 2 (107 mg, 0.224 mmol) following procedure described in Preparation 4, the desired product (40 mg, 0.115 mmol) was obtained as a white solid.

¹H NMR (399 MHz, DMSO-d6) δ 12.22 (s, 1H), 7.96 (d, 1H), 7.67 (s, 1H), 7.59-7.51 (m, 2H), 7.47-7.38 (m, 2H), 7.40-7.31 (m, 1H), 6.99-6.88 (m, 2H), 6.54 (q, 1H), 5.86 (s, 2H), 2.6 (s, 3H), 1.76 (d, 3H).

LC/MS (method B): RT=1.09 min; m/z=346 [M+H]⁺

Examples 1-28 in the following Table 1 were prepared by methods outlined in General Procedure I-III using appropriate commercially available boronate esters and alcohols. The compounds of Example 1, 6, 20 are also included.

TABLE 1
HRMS (TOF, ESI) data

| | | | Calcd Exact | | |
|---|---|---|---|---|---|
| Example | Structure | Mol Formula | Mass | Found m/z | Adduct |
| 1 | 4-methoxy-2-methyl-5-(pyridin-4-yl)-7H- | C13H12N4O | 240.1011 | 241.1082 | [M + H]⁺ |
| | pyrrolo[2,3-d]pyrimidine | | | | |
| 2 | 4-(4-methoxy-2-methyl-7H-pyrrolo[2,3- | C13H13N5O | 255.1120 | 256.1196 | [M + H]⁺ |
| | d]pyridin-5-yl)pyrimidin-2-amine | | | | |
| 3 | 5-(2-fluoropyridin-4-yl)-4-methoxy-2-methyl- | C13H11FN4O | 258.0917 | 259.0996 | [M + H]⁺ |
| | 7H-pyrrolo[2,3-d]pyrimidine | | | | |
| 4 | 4-methoxy-2-methyl-5-(2-methylpyridin-4-yl)- | C14H14N4O | 254.1168 | 255.1238 | [M + H]⁺ |
| | 7H-pyrrolo[2,3-d]pyrimidine | | | | |
| 5 | 2-methyl-5-(2-methylpyridin-4-yl)-4- | C18H16N4OS | 336.1045 | 337.1129 | [M + H]⁺ |
| | (thiophen-3-ylmethoxy)-7H-pyrrolo[2,3- | | | | |
| | d]pyrimidine | | | | |
| 6 | 2-methyl-5-(2-methylpyridin-4-yl)-4-[(3R)- | C19H23N5O | 337.1903 | 338.1982 | [M + H]⁺ |
| | piperidin-3-ylmethoxy]-7H-pyrrolo[2,3- | | | | |
| | d]pyrimidine | | | | |
| 7 | 4-(cyclopropylmethoxy)-2-methyl-5-(pyridin- | C16H16N4O | 280.1324 | 281.1400 | [M + H]⁺ |
| | 4-yl)-7H-pyrrolo[2,3-d]pyrimidine | | | | |
| 8 | 4-(2-cyclopropylethoxy)-2-methyl-5-(pyridin- | C17H18N4O | 294.1481 | 293.1409 | [M − H]⁻ |
| | 4-yl)-7H-pyrrolo[2,3-d]pyrimidine | | | | |
| 9 | 4-[2-(1H-indol-3-yl)ethoxy]-2-methyl-5- | C22H19N5O | 369.1590 | 370.1657 | [M + H]⁺ |
| | (pyridin-4-yl)-7H-pyrrolo[2,3-d]pyrimidine | | | | |
| 10 | 2-methyl-4-(2-phenylethoxy)-5-(pyridin-4-yl)- | C20H18N4O | 330.1481 | 331.1547 | [M + H]⁺ |
| | 7H-pyrrolo[2,3-d]pyrimidine | | | | |
| 11 | 4-(benzyloxy)-2-methyl-5-(pyridin-4-yl)-7H- | C19H16N4O | 316.1324 | 317.1391 | [M + H]⁺ |
| | pyrrolo[2,3-d]pyrimidine | | | | |
| 12 | 2-methyl-5-(pyridin-4-yl)-4-[2-(pyrrolidin-1- | C18H21N5O | 323.1746 | 324.1818 | [M + H]⁺ |
| | yl)ethoxy]-7H-pyrrolo[2,3-d]pyrimidine | | | | |
| 13 | 2-methyl-4-[2-(piperidin-1-yl)ethoxy]-5- | C19H23N5O | 337.1903 | 338.1975 | [M + H]⁺ |
| | (pyridin-4-yl)-7H-pyrrolo[2,3-d]pyrimidine | | | | |
| 14 | 2-methyl-5-(pyridin-4-yl)-4-(tetrahydrofuran- | C17H18N4O2 | 310.1430 | 311.1508 | [M + H]⁺ |
| | 2-ylmethoxy)-7H-pyrrolo[2,3-d]pyrimidine | | | | |
| 15 | 4-(cyclopentylmethoxy)-2-methyl-5-(pyridin- | C18H20N4O | 308.1637 | 309.1711 | [M + H]⁺ |
| | 4-yl)-7H-pyrrolo[2,3-d]pyrimidine | | | | |
| 16 | 2-methyl-4-[(5-methyl-1,2-oxazol-3-yl) | C17H15N5O2 | 321.1226 | 322.1299 | [M + H]⁺ |
| | methoxy]-5-(pyridin-4-yl)-7H-pyrrolo[2,3- | | | | |
| | d]pyrimidine | | | | |
| 17 | 4-[2-methyl-4-(thiophen-3-ylmethoxy)-7H- | C17H15N5OS | 337.0997 | 338.1068 | [M + H]⁺ |
| | pyrrolo[2,3-d]pyrimidin-5-yl]pyridin-2-amine | | | | |
| 18 | 4-[2-methyl-4-(1,3-thiazol-5-ylmethoxy)-7H- | C16H14N6OS | 338.0950 | 339.1025 | [M + H]⁺ |
| | pyrrolo[2,3-d]pyrimidin-5-yl]pyridin-2-amine | | | | |
| 19 | 4-[2-methyl-4-(thiophen-2-ylmethoxy)-7H- | C17H15N5OS | 337.0997 | 338.1072 | [M + H]⁺ |
| | pyrrolo[2,3-d]pyrimidin-5-yl]pyridin-2-amine | | | | |
| 20 | 4-[2-methyl-4-(1-phenylethoxy)-7H- | C20H19N5O | 345.1590 | 346.1654 | [M + H]⁺ |
| | pyrrolo[2,3-d]pyrimidin-5-yl]pyridin-2-amine | | | | |
| 21 | 4-{2-methyl-4-[2-(4-methyl-1,3-thiazol-5- | C18H18N6OS | 366.1263 | 367.1343 | [M + H]⁺ |
| | yl)ethoxy]-7H-pyrrolo[2,3-d]pyrimidin-5- | | | | |
| | yl}pyridin-2-amine | | | | |
| 22 | 4-[2-methyl-4-(pyridin-3-ylmethoxy)-7H- | C18H16N6O | 332.1386 | 333.1453 | [M + H]⁺ |
| | pyrrolo[2,3-d]pyrimidin-5-yl]pyridin-2-amine | | | | |
| 23 | 4-[2-methyl-4-(pyridin-4-ylmethoxy)-7H- | C18H16N6O | 332.1386 | 333.1452 | [M + H]⁺ |
| | pyrrolo[2,3-d]pyrimidin-5-yl]pyridin-2-amine | | | | |
| 24 | 4-(4-{[5-(4-fluorophenyl)-1,2-oxazal-3- | C22H17FN6O2 | 416.1397 | 417.1459 | [M + H]⁺ |
| | yl]methoxy}-2-methyl-7H-pyrrolo[2,3- | | | | |
| | d]pyrimidin-5-yl)pyridin-2-amine | | | | |
| 25 | 4-({[5-(2-aminopyridin-4-yl)-2-methyl-7H- | C20H16N6O | 356.1386 | 357.1464 | [M + H]⁺ |
| | pyrrolo[2,3-d]pyrimidin-4- | | | | |
| | yl]oxy}methyl)benzonitrile | | | | |
| 26 | 4-{4-[(4-methoxybenzyl)oxy]-2-methyl-7H- | C20H19N5O2 | 361.1539 | 360.1463 | [M − H]⁻ |
| | pyrrolo[2,3-d]pyrimidin-5-yl}pyridin-2-amine | | | | |
| 27 | 4-{2-methyl-4-{(4-{propan-2-yl)benzyl]oxy}- | C22H23N5O | 373.1903 | 374.1972 | [M + H]⁺ |
| | 7H-pyrrolo[2,3-d]pyrimidin-5-yl)pyridin-2- | | | | |
| | amine | | | | |
| 28 | 4-[2-methyl-4-(1,3-thiazol-4-ylmethoxy)-7H- | C16H14N6OS | 338.0950 | 339.1019 | [M + H]⁺ |
| | pyrrolo[2,3-d]pyrimidin-5-yl]pyridin-2-amine | | | | |

### General Procedure IV

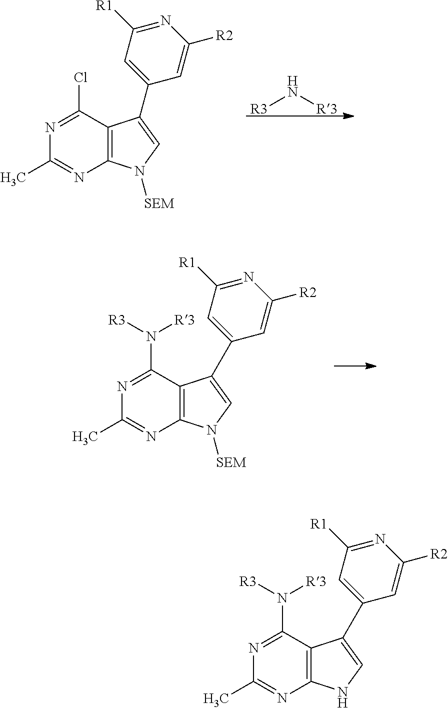

### General Procedure V

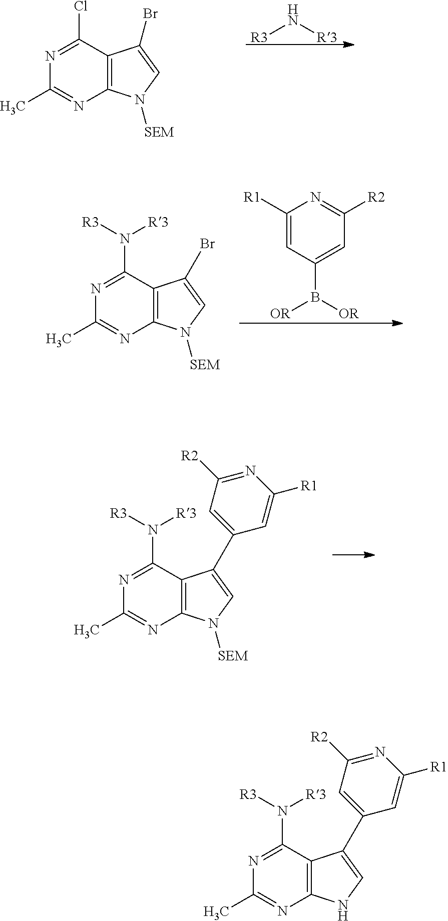

### General Procedure VI

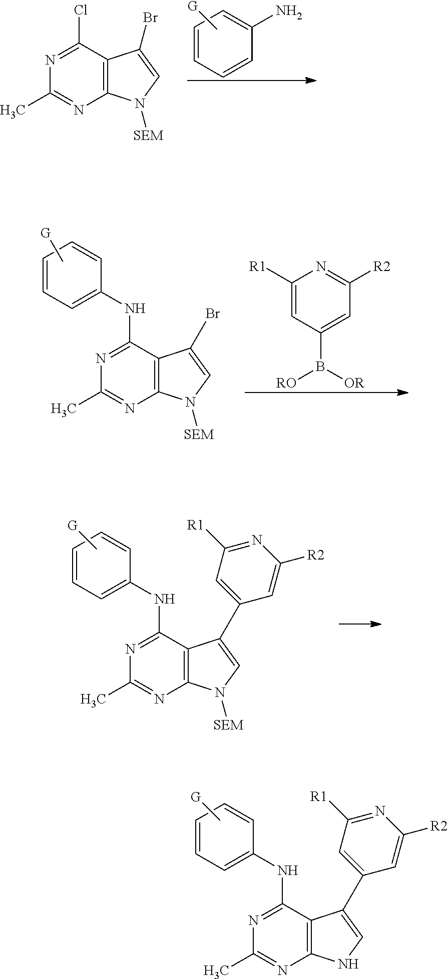

In General Procedures IV, V and VI:
R₁ and R₂ are as defined in formula (I),
R₃ represents a hydrogen atom, a linear or branched (C₁-C₆)alkyl group, —(C₀-C₆)alkylene-Cy₁, —(C₀-C₆)alkylene-Cy₁-Cy₂, —(C₀-C₆)alkylene-Cy₁-O—(C₁-C₆)alkylene-Cy₂, it being understood that Cy₁ and Cy₂, independently of one another, represent a cycloalkyl group, a heterocycloalkyl group, an aryl or an heteroaryl group,
and R′₃ represents a hydrogen atom or a linear or branched (C₁-C₆)alkyl group,
or R₃ and R′₃ form with the nitrogen atom carrying them a heterocycloalkyl or an heteroaryl.
   G represents a group selected from the list of substituents defined in formula (I), it being understood that the phenyl may be substituted by from 1 to 4 independent G groups.

### EXAMPLE 30

### 4-[2-methyl-4-(pyrrolidin-1-yl)-7H-pyrrolo[2,3-d]pyrimidin-5-yl]pyridin-2-amine

### Step 1: 4-[2-methyl-4-(pyrrolidin-1-yl)-7-{[2-(trimethylsilyl)ethoxy]methyl}-7H-pyrrolo[2,3-d]pyrimidin-5-yl]pyridin-2-amine (Preparation 8)

To a solution of 4-(4-chloro-2-methyl-7-{[2-(trimethylsilyl)ethoxy]methyl}-7H-pyrrolo[2,3-d]pyrimidin-5-yl)pyridin-2-amine (Example 20, Step 1) (50 mg, 0.128 mmol) in THF (3 mL) was added pyrrolidine (3 eq). The reaction mixture was heated at 90° C. on a CEM microwave reactor for 1 hour (reaction monitored by LC-MS). The reaction mixture was diluted with DCM (10 mL) and water (10 mL). The organic layer was separated, washed with brine, dried over MgSO₄ and concentrated in vacuo to give the desired product (58 mg, >100%). Purity estimated around 90% by LCMS. The compound was used without further purification.

LC/MS (method A): RT=2.08 mm; m/z=425 [M+H]⁺

### Step 2: 4-[2-methyl-4-(pyrrolidin-1-yl)-7H-pyrrolo[2,3-d]pyrimidin-5-yl]pyridin-2-amine

Starting from the compound obtained in Step 1 (58 mg) following procedure described in Preparation 4, the desired product (23 mg, 0.078 mmol, 61% over two steps) was obtained as a white solid.

¹H NMR (399 MHz, DMSO-d6) δ 11.71 (s, 1H), 7.86 (d, 1H), 7.17 (d, 1H), 6.56-6.44 (m, 2H), 5.89 (s, 2H), 3.31 (m, 4H), 2.41 (s, 3H), 1.72-1.63 (m, 4H)

### EXAMPLE 32

### 5-(2-aminopyridin-4-yl)-N-benzyl-2-methyl-7H-pyrrolo[2,3-d]pyrimidin-4-amine

### Step 1: N-benzyl-5-bromo-2-methyl-7-{[2-(trimethylsilyl)ethoxy]methyl}-7H-pyrrolo[2,3-d]pyrimidin-4-amine

Starting from 5-bromo-4-chloro-2-methyl-7-{[2-(trimethylsilyl)ethoxy]methyl}-7H-pyrrolo[2,3-d]pyrimidin (Example 1, Step 1) (1 g, 2.65 mmol) and phenylmethanamine (4 eq) following procedure described in Preparation 8. The residue was purified via flash chromatography using EtOAc and isohexane as eluent to give the product (1.08 g, 2.41 mmol, 91%) as a clear oil.

¹H NMR (399 MHz, DMSO-d6) δ 7.55 (s, 1H), 7.49-7.26 (m, 5H), 7.04 (t, 1H), 5.51 (s, 2H), 4.85 (d, 2H), 3.62-3.53 (m, 2H), 2.47 (s, 3H), 0.99-0.85 (m, 2H), 0.00 (s, 9H).

LC/MS (method A): RT=2.95 min; m/z=449 [M+H]⁺

### Step 2: 5-(2-aminopyridin-4-yl)-N-benzyl-2-methyl-7-{[2-(trimethylsilyl)ethoxy]methyl}-7H-pyrrolo[2,3-d]pyrimidin-4-amine

Starting from the compound obtained in Step 1 (0.702 g, 1.57 mmol) and 4-(tetramethyl-1,3,2-dioxaborolan-2-yl)pyridin-2-amine (1.1 eq) following procedure described in Preparation 3, the desired product (0.335 g, 0.727 mmol, 46%) was obtained as a light brown oil.

¹H NMR (399 MHz, DMSO-d6) δ 7.97 (dd, 1H), 7.50-7.34 (m, 5H), 7.35-7.26 (m, 1H), 6.65-6.56 (m, 2H), 6.09 (t, 1H), 6.06 (s, 2H), 5.58 (s, 2H), 4.77 (d, 2H), 3.67-3.58 (m, 2H), 2.51 (s, 3H), 0.98-0.84 (m, 2H), 0.00 (s, 9H).

LC/MS (method A): RT=2.33 mm; m/z=461 [M+H]⁺

### Step 3: 5-(2-aminopyridin-4-yl)-N-benzyl-2-methyl-7H-pyrrolo[2,3-d]pyrimidin-4-amine

Starting from the compound obtained in Step 2 (0.335 g, 0.727 mmol) following procedure described in Preparation 4, the desired product (51 mg, 0.154 mmol, 21%) was obtained as a white solid.

¹H NMR (399 MHz, DMSO-d6) δ 11.73 (s, 1H), 7.89 (d, 1H), 7.42-7.28 (m, 4H), 7.29-7.19 (m, 2H), 6.60-6.49 (m, 2H), 5.92 (d, 3H), 4.70 (d, 2H), 2.42 (s, 3H).

LC/MS (method A): RT=1.65 min; m/z=331 [M+H]⁺

### EXAMPLE 52

### 5-(2-aminopyridin-4-yl)-N-(2,6-difluorobenzyl-2-methyl-7H-pyrrolo[2,3-d]pyrimidin-4-amine

### Step-1: 5-bromo-N-[(2,6-difluorophenyl)methyl]-2-methyl-7-{[2-(trimethylsilyl)ethoxy]methyl}-7H-pyrrolo[2,3-d]pyrimidin-4-amine

Starting from 5-bromo-4-chloro-2-methyl-7-{[2-(trimethylsilyl)ethoxy]methyl}-7H-pyrrolo[2,3-d]pyrimidine (Example 1, Step 1) (1.2 g, 3.19 mmol) and (2,6-difluorophenyl)methanamine (4 eq) following procedure described in Preparation 8. The residue was purified via flash chromatography using EtOAc and isohexane as eluent to give the desired product as a clear oil.

¹H NMR (399 MHz, DMSO-d6) δ 7.56 (s, 1H), 7.46 (tt, 1H), 7.24-7.11 (m, 2H), 6.81 (t, 1H), 5.51 (s, 2H), 4.92 (d, 2H), 3.62-3.53 (m, 2H), 2.49 (s, 3H), 0.97-0.85 (m, 2H), 0.00 (s, 9H).

LC/MS (method A): RT=2.96 min; m/z=485 [M+H]⁺

### Step 2: 5-(2-aminopyridin-4-yl)-N-(2,6-difluorobenzyl)-2-methyl-7-{[2-(trimethylsilyl) ethoxy]methyl}-7H-pyrrolo[2,3-d]pyrimidin-4-amine

Starting from the compound obtained in Step 1 (1 g, 2.07 mmol) and 4-(tetramethyl-1,3,2-dioxaborolan-2-yl)pyridin-2-amine (1.1 eq) following procedure described in Preparation 3, the desired product (0.422 g. 0.849 mmol, 41%) was obtained as a light brown oil.

¹H NMR (399 MHz, DMSO-d6) δ 7.99 (dd, 1H), 7.52-7.39 (m, 2H), 7.22-7.11 (m, 2H), 6.61-6.53 (m, 2H), 6.05 (d, 3H), 5.57 (s, 2H), 4.85 (d, 2H), 3.66-3.57 (m, 2H), 2.53 (s, 3H), 1.00-0.86 (m, 2H), 0.00 (s, 9H).

LC/MS (method B): RT=1.32 min; m/z=497 [M+H]⁺

### Step 3: 5-(2-aminopyridin-4-yl)-N-(2,6-difluorobenzyl)-2-methyl-7H-pyrrolo[2,3-d]pyrimidin-4-amine

Starting from the compound obtained in Step 2 (0.422 g, 0.849 mmol) following procedure described in Preparation 4, the product (0.104 g, 0.284 mmol, 33%) was obtained as a white solid.

¹H NMR (399 MHz, DMSO-d6) δ 11.74 (s, 1H), 7.90 (d, 1H), 7.37 (tt, 1H), 7.24 (d, 1H), 7.09 (t, 2H), 6.54-6.45 (m, 2H), 5.93 (s, 2H), 5.85 (t, 1H), 4.77 (d, 2H), 2.43 (s, 3H).

LC/MS (method B): RT=0.96 min; m/z=367 [M+H]⁺

### EXAMPLE 129

### 5-(2-aminopyridin-4-yl)-2-methyl-N-phenyl-7H-pyrrolo[2,3-d]pyrimidin-4-amine

Step 1: 5-bromo-2-methyl-N-phenyl-{[2-(trimethylsilyl)ethoxy]methyl}-7H-pyrrolo [2,3-d]pyrimidin-4-amine (Preparation 9)

To a solution of 5-bromo-4-chloro-2-methyl-7-{[2-(trimethylsilyl)ethoxy]methyl}-7H-pyrrolo[2,3-d]pyrimidine (Example 1, Step 1) (0.2 g, (0.53 mmol) in DMF (2 mL) was added aniline (1.2 eq) followed by t-BuOK (2 eq) at room temperature under N₂. The reaction mixture was stirred at room temperature for 2 hours. The reaction mixture was diluted with water (10 mL) and EtOAc (20 mL). The organic layer was separated, washed with brine, dried over MgSO₄ and concentrated in vacuo. The residue was purified via flash chromatography using EtOAc and isohexane as eluent to give the product (0.109 g, 0.251 mmol, 47%) as a clear oil.

LC/MS (method B): RT=1.68 min; m/z=433 [M+H]⁺

Step 2: tert-butyl N-{4-[2-methyl-4-(phenylamino)-7-{[2-(trimethylsilyl)ethoxy]methyl}-7H-pyrrolo[2,3-d]pyrimidin-5-yl]pyridin-2-yl}carbamate

Starting from the compound obtained in Step 1 (0.109 g, 0.251 mmol) and tert-butyl N-[4-(tetramethyl-1,3,2-dioxaborolan-2-yl)pyridin-2-yl]carbamate (1.2 eq) following procedure described in Preparation 3, the product (0.118 g, 0.215 mmol, 86%) was obtained as clear oil.

LC/MS (method B): RT=1.68 min; m/z=547 [M+H]⁺

### Step 3: 5-(2-aminopyridin-4-yl)-2-methyl-N-phenyl-7H-pyrrolo[2,3-d]pyrimidin-4-amine

Starting from the compound obtained in Step 2 (0.118 g, 0.215 mmol) following procedure described in Preparation 7, the desired product (37 mg, 0.117 mmol, 54%) was obtained as a pale yellow solid.

¹H NMR (399 MHz, DMSO-d6) δ 12.00 (d, 1H), 8.00 (d, 1H), 7.72-7.65 (m, 3H), 7.45 (d, 1H), 7.35-7.28 (m, 2H), 7.00 (m, 1H), 6.71 (dd, 1H), 6.63 (d, 1H), 6.25 (s, 2H), 2.53 (s, 3H).

LC/MS (method B): RT=0.87 min; m/z=317 [M+H]⁺

Examples 29-146 in the following Table 2 were prepared by methods outlined in General Procedure IV-VI using appropriate commercially available boronate esters and amines. The compounds of Example 30, 32, 129 are also included.

TABLE 2
HRMS (TOF, ESI) data

| | | | Calcd Exact | | |
|---|---|---|---|---|---|
| Example | Structure | Mol Formula | Mass | Found m/z | Adduct |
| 29 | 5-(2-aminopyridin-4-yl)-N,N,2-trimethyl- | C14H16N6 | 268.1436 | 269.1519 | [M + H]⁺ |
| | 7H-pyrrolo[2,3-d]pyrimidin-4-amine | | | | |
| 30 | 4-[2-methyl-4-(pyrrolidin-1-yl)-7H- | C16H18N6 | 294.1593 | 295.1672 | [M + H]⁺ |
| | pyrrolo[2,3-d]pyrimidin-5-yl]pyridin-2- | | | | |
| | amine | | | | |
| 31 | 4-{4-[3-(dimethylamino)pyrrolidin-1-yl]-2- | C18H23N7 | 337.2015 | 338.2085 | [M + H]⁺ |
| | methyl-7H-pyrrolo[2,3-d]pyrimidin-5- | | | | |
| | yl}pyridin-2-amine | | | | |
| 32 | 5-(2-aminopyridin-4-yl)-N-benzyl-2- | C19H18N6 | 330.1593 | 331.1661 | [M + H]⁺ |
| | methyl-7H-pyrrolo[2,3-d]pyrimidin-4- | | | | |
| | amine | | | | |
| 33 | 4-[2-methyl-4-(4-methypiperazin-1-yl)-7H- | C17H21N7 | 323.1858 | 324.1932 | [M + H]⁺ |
| | pyrrolo[2,3-d]pyrimidin-5-yl]pyridin-2- | | | | |
| | amine | | | | |
| 34 | 5-(2-aminopyridin-4-yl)-2-methyl-N- | C18H17N7 | 331.1545 | 332.1612 | [M + H]⁺ |
| | (pyridin-3-ylmethyl)-7H-pyrrolo[2,3- | | | | |
| | d]pyrimidin-4-amine | | | | |
| 35 | 5-(2-aminopyridin-4-yl)-N-(furan-3- | C17H16N6O | 320.1386 | 321.1466 | [M + H]⁺ |
| | ylmethyl)-2-methyl-7H-pyrrolo[2,3- | | | | |
| | d]pyrimidin-4-amine | | | | |
| 36 | 5-(2-aminopyridin-4-yl)-2-methyl-N- | C17H16N6S | 336.1157 | 337.1231 | [M + H]⁺ |
| | (thiophen-3-ylmethyl)-7H-pyrrolo[2,3- | | | | |
| | d]pyrimidin-4-amine | | | | |
| 37 | 5-(2-aminopyridin-4-yl)-2-methyl-N- | C17H16N6S | 336.1157 | 337.1222 | [M + H]⁺ |
| | (thiophen-2-ylmethyl)-7H-pyrrolo[2,3- | | | | |
| | d]pyrimidin-4-amine | | | | |
| 38 | 5-(2-aminopyridin-4-yl)-2-methyl-N-[(1- | C17H18N8 | 334.1654 | 335.1722 | [M + H]⁺ |
| | methyl-1H-pyrazol-5-yl)methyl]-7H- | | | | |
| | pyrrolo[2,3-d]pyrimidin-4-amine | | | | |
| 39 | 5-(2-aminopyridin-4-yl)-2-methyl-N-(1,3- | C16H15N7S | 337.1110 | 338.1179 | [M + H]⁺ |
| | thiazol-2-ylmethyl)-7H-pyrrolo[2,3- | | | | |
| | d]pyrimidin-4-amine | | | | |
| 40 | 5-(2-aminopyridin-4-yl)-2-methyl-N-(1,3- | C16H15N7S | 337.1110 | 338.1189 | [M + H]⁺ |
| | thiazol-4-ylmethyl)-7H-pyrrolo[2,3-d] | | | | |
| | pyrimidin-4-amine | | | | |
| 41 | 5-(2-aminopyridin-4-yl)-2-methyl-N-(1,3- | C16H15N7S | 337.1110 | 338.1179 | [M + H]⁺ |
| | thiazol-5-ylmethyl)-7H-pyrrolo[2,3-d] | | | | |
| | pyrimidin-4-amine | | | | |
| 42 | N-benzyl-2-methyl-5-(pyridin-4-yl)-7H- | C19H17N5 | 315.1484 | 316.1547 | [M + H]⁺ |
| | pyrrolo[2,3-d]pyrimidin-4-amine | | | | |
| 43 | N-benzyl-2-methyl-5-(2-methylpyridin-4- | C20H19N5 | 329.1640 | 330.1709 | [M + H]⁺ |
| | yl)-7H-pyrrolo[2,3-d]pyrimidin-4-amine | | | | |
| 44 | 2-methyl-5-(pyridin-4-yl)-N-(thiophen-3- | C17H15N5S | 321.1048 | 322.1121 | [M + H]⁺ |
| | ylmethyl)-7H-pyrrolo[2,3-d]pyrimidin-4- | | | | |
| | amine | | | | |
| 45 | 2-methyl-5-(2-methylpyridin-4-yl)-N- | C18H17N5S | 335.1205 | 336.1283 | [M + H]⁺ |
| | (thiophen-3-ylmethyl)-7H-pyrrolo[2,3-d] | | | | |
| | pyrimidin-4-amine | | | | |
| 46 | 2-methyl-N-[(5-methyl-1,2-oxazol-3- | C17H16N6O | 320.1386 | 321.1450 | [M + H]⁺ |
| | yl)methyl]-5-(pyridin-4-yl)-7H-pyrrolo[2,3-d] | | | | |
| | pyrimidin-4-amine | | | | |
| 47 | 2-methyl-N-[(5-methyl-1,2-oxazol-3-yl) | C18H18N6O | 334.1542 | 335.1613 | [M + H]⁺ |
| | methyl]-5-(2-methylpyridin-4-yl)-7H- | | | | |
| | pyrrolo[2,3-d]pyrimidin-4-amine | | | | |
| 48 | 5-(2-aminopyridin-4-yl)-N- | C19H24N6 | 336.2062 | 337.2134 | [M + H]⁺ |
| | (cyclohexylmethyl)-2-methyl-7H-pyrrolo | | | | |
| | [2,3-d]pyrimidin-4-amine | | | | |
| 49 | 5-(2-aminopyridin-4-yl)-2-methyl-N-(1- | C20H20N6 | 344.1749 | 345.1814 | [M + H]⁺ |
| | phenylethyl)-7H-pyrrolo[2,3-d]pyrimidin-4- | | | | |
| | amine | | | | |
| 50 | 5-(2-aminopyridin-4-yl)-N-(3- | C19H17FN6 | 348.1499 | 349.1567 | [M + H]⁺ |
| | fluorobenzyl)-2-methyl-7H-pyrrolo[2,3- | | | | |
| | d]pyrimidin-4-amine | | | | |
| 51 | 5-(2-aminopyridin-4-yl)-N-(2- | C19H17FN6 | 348.1499 | 347.1430 | [M − H]⁻ |
| | fluorobenzyl)-2-methyl-7H-pyrrolo[2,3- | | | | |
| | d]pyrimidin-4-amine | | | | |
| 52 | 5-(2-aminopyridin-4-yl)-N-(2,6- | C19H16F2N6 | 366.1405 | 365.1341 | [M − H]⁻ |
| | difluorobenzyl)-2-methyl-7H-pyrrolo[2,3- | | | | |
| | d]pyrimidin-4-amine | | | | |
| 53 | 5-(2-aminopyridin-4-yl)-2-methyl-N- | C18H17N7 | 331.1545 | 330.1471 | [M − H]⁻ |
| | (pyridin-2-ylmethyl)-7H-pyrrolo[2,3- | | | | |
| | d]pyrimidin-4-amine | | | | |
| 54 | 5-(2-aminopyridin-4-yl)-N-(4- | C19H17FN6 | 348.1499 | 347.1416 | [M − H]⁻ |
| | fluorobenzyl)-2-methyl-7H-pyrrolo[2,3- | | | | |
| | d]pyrimidin-4-amine | | | | |
| 55 | 5-(2-aminopyridin-4-yl)-N-(2- | C20H20N6O | 360.1699 | 359.1611 | [M − H]⁻ |
| | methoxybenzyl)-2-methyl-7H-pyrrolo[2,3- | | | | |
| | d]pyrimidin-4-amine | | | | |
| 56 | 5-(2-aminopyridin-4-yl)-2-methyl-N-(2- | C20H20N6 | 344.1749 | 343.1675 | [M − H]⁻ |
| | methylbenzyl)-7H-pyrrolo[2,3-d]pyrimidin- | | | | |
| | 4-amine | | | | |
| 57 | 5-(2-aminopyridin-4-yl)-N-(2- | C19H17ClN6 | 364.1203 | 363.1139 | [M − H]⁻ |
| | chlorobenzyl)-2-methyl-7H-pyrrolo[2,3- | | | | |
| | d]pyrimidin-4-amine | | | | |
| 58 | 5-(2-aminopyridin-4-yl)-N-(2-chloro-6- | C20H19ClN6 | 378.1360 | 377.1292 | [M − H]⁻ |
| | methylbenzyl)-2-methyl-7H-pyrrolo[2,3- | | | | |
| | d]pyrimidin-4-amine | | | | |
| 59 | 5-(2-aminopyridin-4-yl)-2-methyl-N-[(5- | C17H17N7O | 335.1495 | 334.1417 | [M − H]⁻ |
| | methyl-1,2-oxazol-3-yl)methyl]-7H- | | | | |
| | pyrrolo[2,3-d]pyrimidin-4-amine | | | | |
| 60 | 5-(2-aminopyridin-4-yl)-2-methyl-N-[(3- | C19H19N7 | 345.1702 | 344.1630 | [M − H]⁻ |
| | methylpyridin-2-yl)methyl]-7H-pyrrolo[2,3- | | | | |
| | d]pyrimidin-4-amine | | | | |
| 61 | 5-(2-aminopyridin-4-yl)-N-(2,6- | C19H16Cl2N6 | 398.0813 | 397.0746 | [M − H]⁻ |
| | dichlolorobenzyl)-2-methyl-7H-pyrrolo[2,3- | | | | |
| | d]pyrimidin-4-amine | | | | |
| 62 | 5-(2-aminopyridin-4-yl)-N-(2-chloro-6- | C19H16ClFN6 | 382.1109 | 381.1045 | [M − H]⁻ |
| | fluorobenzyl)-2-methyl-7H-pyrrolo[2,3- | | | | |
| | d]pyrimidin-4-amine | | | | |
| 63 | 5-(2-aminopyridin-4-yl)-N-(2,4- | C19H16F2N6 | 366.1405 | 365.1323 | [M − H]⁻ |
| | difluorobenzyl)-2-methyl-7H-pyrrolo[2,3- | | | | |
| | d]pyrimidin-4-amine | | | | |
| 64 | 5-(2-aminopyridin-4-yl)-2-methyl-N-[2- | C20H17F3N6 | 398.1467 | 397.1402 | [M − H]⁻ |
| | (trifluoromethyl)benzyl]-7H-pyrrolo[2,3- | | | | |
| | d]pyrimidin-4-amine | | | | |
| 65 | 5-(2-aminopyridin-4-yl)-N- | C16H18N6 | 294.1593 | 293.1535 | [M − H]⁻ |
| | (cyclopropymethyl)-2-methyl-7H- | | | | |
| | pyrrolo[2,3-d]pyrimidin-4-amine | | | | |
| 66 | 5-(2-aminopyridin-4-yl)-2-methyl-N-[1- | C19H19N7 | 345.1702 | 344.1636 | [M − H]⁻ |
| | (pyridin-2-yl)ethyl]-7H-pyrrolo[2,3- | | | | |
| | d]pyrimidin-4-amine | | | | |
| 67 | 5-(2-aminopyridin-4-yl)-2-methyl-N-[(1S)- | C20H20N6 | 344.1749 | 343.1688 | [M − H]⁻ |
| | 1-phenylethyl]-7H-pyrrolo[2,3-d]pyrimidin- | | | | |
| | 4-amine | | | | |
| 68 | 5-(2-aminopyridin-4-yl)-2-methyl-N-[(1R)- | C20H20N6 | 344.1749 | 343.1680 | [M − H]⁻ |
| | 1-phenylethyl]-7H-pyrrolo[2,3-d]pyrimidin- | | | | |
| | 4-amine | | | | |
| 69 | 5-(2-aminopyridin-4-yl)-N-(2-fluoro-6- | C20H19FN6O | 378.1604 | 377.1534 | [M − H]⁻ |
| | methoxybenzyl)-2-methyl-7H-pyrrolo[2,3- | | | | |
| | d]pyrimidin-4-amine | | | | |
| 70 | 5-(2-aminopyridin-4-yl)-N-(2-fluoro-6- | C20H19FN6 | 362.1655 | 361.1593 | [M − H]⁻ |
| | methylbenzyl)-2-methyl-7H-pyrrolo[2,3- | | | | |
| | d]pyrimidin-4-amine | | | | |
| 71 | 5-(2-aminopyridin-4-yl)-N-[(3- | C18H16FN7 | 349.1451 | 348.1370 | [M − H]⁻ |
| | fluoropyridin-2-yl)methyl]-2-methyl-7H- | | | | |
| | pyrrolo[2,3-d]pyrimidin-4-amine | | | | |
| 72 | 5-(2-aminopyridin-4-yl)-N-(1H-indol-6- | C21H19N7 | 369.1702 | 368.1629 | [M − H]⁻ |
| | ylmethyl)-2-methyl-7H-pyrrolo[2,3- | | | | |
| | d]pyrimidin-4-amine | | | | |
| 73 | 5-(2-aminopyridin-4-yl)-2-methyl-N-(2,3,5- | C19H15F3N6 | 384.1310 | 383.1246 | [M − H]⁻ |
| | trifluorobenzyl)-7H-pyrrolo[2,3- | | | | |
| | d]pyrimidin-4-amine | | | | |
| 74 | 5-(2-aminopyridin-4-yl)-N-(2,3- | C19H16F2N6 | 366.1405 | 365.1328 | [M − H]⁻ |
| | difluorobenzyl)-2-methyl-7H-pyrrolo[2,3- | | | | |
| | d]pyrimidin-4-amine | | | | |
| 75 | 5-(2-ammopyridin-4-yl)-N-[4-fluoro-2- | C20H16F4N6 | 416.1373 | 415.1297 | [M − H]⁻ |
| | (trifluoromethyl)benzyl]-2-methyl-7H- | | | | |
| | pyrrolo[2,3-d]pyrimidin-4-amine | | | | |
| 76 | 5-(2-aminopyridin-4-yl)-N-[(1R)-2,3- | C21H20N6 | 356.1749 | 355.1685 | [M − H]⁻ |
| | dihydro-1H-inden-1-yl]-2-methyl-7H- | | | | |
| | pyrrolo[2,3-d]pyrimidin-4-amine | | | | |
| 77 | 5-(2-aminopyridin-4-yl)-N-[(1S)-2,3- | C21H20N6 | 356.1749 | 355.1677 | [M − H]⁻ |
| | dihydro-1H-inden-1-yl]-2-methyl-7H- | | | | |
| | pyrrolo[2,3-d]pyrimidin-4-amine | | | | |
| 78 | 5-(2-aminopyridin-4-yl)-2-methyl-N-{[3- | C19H16F3N7 | 399.1419 | 398.1370 | [M − H]⁻ |
| | (trifluoromethyl)pyridin-2-yl]methyl}-7H- | | | | |
| | pyrrolo[2,3-d]pyrimidin-4-amine | | | | |
| 79 | 5-(2-aminopyridin-4-yl)-N-(2- | C21H22N6O | 374.1855 | 373.1783 | [M − H]⁻ |
| | ethoxybenzyl)-2-methyl-7H-pyrrolo[2,3- | | | | |
| | d]pyrimidin-4-amine | | | | |
| 80 | 5-(2-aminopyridin-4-yl)-N-[2-methoxy-6- | C21H19F3N6O | 428.1572 | 427.1491 | [M − H]⁻ |
| | (trifluoromethyl)benzyl]-2-methyl-7H- | | | | |
| | pyrrolo[2,3-d]pyrimidin-4-amine | | | | |
| 81 | 5-(2-aminopyridin-4-yl)-N-(2,3- | C19H16Cl2N6 | 398.0813 | 397.0744 | [M − H]⁻ |
| | dichlorbenzyl)-2-methyl-7H-pyrrolo[2,3- | | | | |
| | d]pyrimidin-4-amine | | | | |
| 82 | 5-(2-aminopyridin-4-yl)-N-[1-(2,6- | C20H18F2N6 | 380.1561 | 379.1500 | [M − H]⁻ |
| | difluorophenyl)ethyl]-2-methyl-7H- | | | | |
| | pyrrolo[2,3-d]pyrimidin-4-amine | | | | |
| 83 | 5-(2-aminopyridin-4-yl)-N-[(1R,2R,4S)- | C19H22N6 | 334.1906 | 333.1845 | [M − H]⁻ |
| | bicyclo[2.2.1]hept-2-yl]-2-methyl-7H- | | | | |
| | pyrrolo[2,3-d]pyrimidin-4-amine | | | | |
| 84 | 5-(2-aminopyridin-4-yl)-N-(4-fluoro-2- | C20H19FN6O | 378.1604 | 377.1540 | [M − H]⁻ |
| | methoxybenzyl)-2-methyl-7H-pyrrolo[2,3- | | | | |
| | d]pyrimidin-4-amine | | | | |
| 85 | 5-(2-aminopyridin-4-yl)-N-[(1R)-1-(2- | C21H22N6O | 374.1855 | 373.1791 | [M − H]⁻ |
| | methoxyphenyl)ethyl]-2-methyl-7H- | | | | |
| | pyrrolo[2,3-d]pyrimidin-4-amine | | | | |
| 86 | 5-(2-aminopyridin-4-yl)-N-[(1R)-1-(2- | C20H19FN6 | 362.1655 | 361.1593 | [M − H]⁻ |
| | fluorophenyl)ethyl]-2-methyl-7H- | | | | |
| | pyrrolo[2,3-d]pyrimidin-4-amine | | | | |
| 87 | 5-(2-aminopyridin-4-yl)-2-methyl-N-{[5- | C22H19N7S | 413.1423 | 452.1358 | [M − H]⁻ |
| | (pyridin-2-yl)thiophen-2-yl]methyl}-7H- | | | | |
| | pyrrolo[2,3-d]pyrimidin-4-amine | | | | |
| 88 | 5-(2-aminopyridin-4-yl)-2-methyl N-[(3- | C22H19N7O | 397.1651 | 396.1584 | [M − H]⁻ |
| | phenyl-1,2-oxazol-5-yl)methyl]-7H- | | | | |
| | pyrrolo[2,3-d]pyrimidin-4-amine | | | | |
| 89 | 5-(2-aminopyridin-4-yl)-2-methyl-N-[2- | C20H17F3N6O | 414.1416 | 413.1342 | [M − H]⁻ |
| | (trifluoromethoxy)benzyl]-7H-pyrrolo[2,3- | | | | |
| | d]pyrimidin-4-amine | | | | |
| 90 | 5-(2-aminopyridin-4-yl)-N-[(1R,2R)-2- | C18H19N7O | 428.2325 | 427.2252 | [M − H]⁻ |
| | (benzyloxy)cyclohexyl]-2-methyl-7H- | | | | |
| | pyrrolo[2,3-d]pyrimidin-4-amine | | | | |
| 91 | 5-(2-aminopyridin-4-yl)-2-methyl-N-[(1R)- | C22H22N6 | 370.1906 | 369.1829 | [M − H]⁻ |
| | 1,2,3,4-tetrahydronaphthalen-1-yl]-7H- | | | | |
| | pyrrolo[2,3-d]pyrimidin-4-amine | | | | |
| 92 | 5-(2-aminopyridin-4-yl)-N-(2,5- | C19H16Cl2N6 | 398.0813 | 397.0746 | [M − H]⁻ |
| | dichlorobenzyl)-2-methyl-7H-pyrrolo[2,3- | | | | |
| | d]pyrimidin-4-amine | | | | |
| 93 | 5-(2-aminopyridin-4-yl)-N-cyclohexyl-2- | C18H22N6 | 322.1906 | 321.1830 | [M − H]⁻ |
| | methyl-7H-pyrrolo[2,3-d]pyrimidin-4- | | | | |
| | amine | | | | |
| 94 | 5-(2-aminopyridin-4-yl)-N-(3-chloro-2- | C20H19ClN6 | 378.1360 | 377.1288 | [M − H]⁻ |
| | methylbenzyl)-2-methyl-7H-pyrrolo[2,3- | | | | |
| | d]pyrimidin-4-amine | | | | |
| 95 | 5-(2-aminopyridin-4-yl)-N-[(3,5-dimethyl- | C18H19N7O | 349.1651 | 348.1587 | [M − H]⁻ |
| | 1,2-oxazol-4-yl)methyl]-2-methyl-7H- | | | | |
| | pyrrolo[2,3-d]pyrimidin-4-amine | | | | |
| 96 | 4-[4-(3,4-dihydroisoquinolin-2(1H)-yl)-2- | C21H20N6 | 356.1749 | 355.1680 | [M − H]⁻ |
| | methyl-7H-pyrrolo[2,3-d]pyrimidin-5- | | | | |
| | yl]pyridin-2-amine | | | | |
| 97 | 4-[2-methyl-4-{3-methylpiperidin-1-yl)-7H- | C18H22N6 | 322.1906 | 323.1978 | [M − H]⁺ |
| | pyrrolo[2,3-d]pyrimidin-5-yl]pyridin-2- | | | | |
| | amine | | | | |
| 98 | 5-(2-aminopyridin-4-yl)-N-[(3- | C19H19N7O | 361.1651 | 362.1728 | [M − H]⁺ |
| | methoxypyridin-2-yl)methyl]-2-methyl-7H- | | | | |
| | pyrrolo[2,3-d]pyrimidin-4-amine | | | | |
| 99 | 5-(2-aminopyridin-4-yl)-N-(2,3-dihydro- | C21H20N6 | 356.1749 | 357.1823 | [M + H]⁺ |
| | 1H-inden-2-yl)-2-methyl-7H-pyrrolo[2,3- | | | | |
| | d]pyrimidin-4-amine | | | | |
| 100 | 5-(2-aminopyridin-4-yl)-2-methyl-N-(3,3,3- | C15H15F3N6 | 336.1310 | 335.1250 | [M − H]⁻ |
| | trifluoropropyl)-7H-pyrrolo[2,3- | | | | |
| | d]pyrimidin-4-amine | | | | |
| 101 | 4-[4-(3-azaspiro[5.5]undec-3-yl)-2-methyl- | C22H28N6 | 376.2375 | 375.2309 | [M − H]⁻ |
| | 7H-pyrrolo[2,3-d]pyrimidin-5-yl]pyridin-2- | | | | |
| | amine | | | | |
| 102 | 4-[2-methyl-4-(8-methyl-2-azaspiro[4.5] | C22H28N6 | 376.2375 | 377.2441 | [M − H]⁺ |
| | dec-2-yl)-7H-pyrrolo[2,3-d]pyrimidin-5- | | | | |
| | yl]pyridin-2-amine | | | | |
| 103 | 4-[2-methyl-4-(2-phenylazetidin-1-yl)-7H- | C21H20N6 | 356.1749 | 357.1821 | [M − H]⁺ |
| | pyrrolo[2,3-d]pyrimadin-5-yl]pyridin-2- | | | | |
| | amine | | | | |
| 104 | 4-[2-methyl-4-(octahydroisoquinolin-2(1H)- | C21H26N6 | 362.2219 | 363.2285 | [M − H]⁺ |
| | yl)-7H-pyrrolo[2,3-d]pyrimidin-5- | | | | |
| | yl]pyridin-2-amine | | | | |
| 105 | 4-{2-methyl-4-[4- | C18H19F3N6 | 376.1623 | 375.1563 | [M − H]⁻ |
| | (trifluoromethyl)piperidin-1-yl]-7H- | | | | |
| | pyrrolo[2,3-d]pyrimidin-5-yl}pyridin-2- | | | | |
| | amine | | | | |
| 106 | 5-(2-aminopyridin-4-yl)-N-[(1S)-1-(2- | C21H22N6O | 374.1855 | 375.1920 | [M − H]⁺ |
| | methoxyphenyl)ethyl]-2-methyl-7H- | | | | |
| | pyrrolo[2,3-d]pyrimidin-4-amine | | | | |
| 107 | 4-{2-methyl-4-[2- | C17H17F3N6 | 362.1467 | 363.1528 | [M − H]⁺ |
| | (trifluoromethyl)pyrrolidin-1-yl]-7H- | | | | |
| | pyrrolo[2,3-d]pyrimidin-5-yl}pyridin-2- | | | | |
| | amine | | | | |
| 108 | 4-[4-(6,7-dihydrothieno[3,2-c]pyridin- | C19H18N6S | 362.1314 | 363.1393 | [M − H]⁺ |
| | 5(4H)-yl)-2-methyl-7H-pyrrolo[2,3- | | | | |
| | d]pyrimidin-5-yl]pyridin-2-amine | | | | |
| 109 | 4-[4-(2-azaspiro[3.5]non-2-yl)-2-methyl- | C20H24N6 | 348.2062 | 347.1993 | [M − H]⁻ |
| | 7H-pyrrolo[2,3-d]pyrimidin-5-yl]pyridin-2- | | | | |
| | amine | | | | |
| 110 | 4-{2-methyl-4-[(4aR,8aR)- | C21H26N6 | 362.2219 | 361.2154 | [M − H]⁻ |
| | octahydroisoquinolin-2(1H-yl]-7H- | | | | |
| | pyrrolo[2,3-d]pyrimidin-5-yl}pyridin-2- | | | | |
| | amine | | | | |
| 111 | 4-{2-methyl-4-[(4aR,8aS)-octahydro- | C21H26N6 | 362.2219 | 361.2145 | [M − H]⁻ |
| | isoquinolin-2(1H)-yl]-7H-pyrrolo[2,3- | | | | |
| | d]pyrimidin-5-yl}pyridin-2-amine | | | | |
| 112 | 5-(2-aminopyridin-4-yl)-N-(2,3-dihydro-1- | C21H20N6O | 372.1699 | 373.1761 | [M − H]⁺ |
| | benzofuran-3-ylmethyl)-2-methyl-7H- | | | | |
| | pyrrolo[2,3-d]pyrimidin-4-amine | | | | |
| 113 | 5-(2-aminopyridin-4-yl)-N-[1-(3- | C20H21N7O | 375.1808 | 376.1876 | [M − H]⁺ |
| | methoxypyridin-2-yl)ethyl]-2-methyl-7H- | | | | |
| | pyrrolo[2,3-d]pyrimidin-4-amine | | | | |
| 114 | 4-[4-(3,4-dihydroisoquinolin-2(1H)-yl)-2- | C21H21N7 | 371.1858 | 370.1786 | [M − H]⁻ |
| | methyl-7H-pyrrolo[2,3-d]pyrimidin-5- | | | | |
| | yl]pyridine-2,6-diamine | | | | |
| 115 | 4-{4-[(2,6-difluorobenzyl)amino]-2-methyl- | C19H17F2N7 | 381.1513 | 382.1556 | [M − H]⁺ |
| | 7H-pyrrolo[2,3-d]pyrimidin-5-yl}pyridine- | | | | |
| | 2,6-diamine | | | | |
| 116 | 4-{4-[(2-fluoro-6-methoxybenzyl)amino]-2- | C20H20FN7O | 393.1713 | 394.1774 | [M − H]⁺ |
| | methyl-7H-pyrrolo[2,3-d]pyrimidin-5- | | | | |
| | yl}pyridine-2,6-diamine | | | | |
| 117 | 4-(4-{[(1S}-1-(2- | C21H23N7O | 389.1964 | 390.2023 | [M − H]⁺ |
| | methoxyphenyl)ethyl]amino}-2-methyl-7H- | | | | |
| | pyrrolo[2,3-d]pyrimidin-5-yl)pyridine-2,6- | | | | |
| | diamine | | | | |
| 118 | 4-{4-[(1R)-2,3-dihydro-1H-inden-1- | C21H21N7 | 371.1858 | 372.1936 | [M − H]⁺ |
| | ylamino]-2-methyl-7H-pyrrolo[2,3- | | | | |
| | d]pyrimidin-5-yl}pyridine-2,6-diamine | | | | |
| 119 | 5-(2-aminopyridin-4-yl)-N-[(3,5- | C18H15F2N7 | 367.1357 | 368.1421 | [M − H]⁺ |
| | difluoropyridin-4-yl)methyl]-2-methyl-7H- | | | | |
| | pyrrolo[2,3-d]pyrimidin-4-amine | | | | |
| 120 | 5-(2-aminopyridin-4-yl)-N-(2,6- | C20H18F2N6 | 380.1561 | 381.1629 | [M − H]⁺ |
| | diflurobenzyl)-N,2-dimethyl-7H- | | | | |
| | pyrrolo[2,3-d]pyrimidin-4-amine | | | | |
| 121 | 5-(2-aminopyridin-4-yl)-N-[1-(3- | C20H20FN7 | 377.1764 | 378.1836 | [M − H]⁺ |
| | fluoropyridin-2-yl)propyl]-2-methyl-7H- | | | | |
| | pyrrolo[2,3-d]pyrimidin-4-amine | | | | |
| 122 | 5-(2-aminopyridin-4-yl)-N-[(1S)-1-(3- | C19H18FN7 | 363.1608 | 364.1636 | [M − H]⁺ |
| | fluoropyridin-2-yl)ethyl]-2-methyl-7H- | | | | |
| | pyrrolo[2,3-d]pyrimidin-4-amine | | | | |
| 123 | 5-(2-aminopyridin-4-yl)-N-(2,2-difluoro-2- | C20H18F2N6 | 380.1561 | 381.1635 | [M − H]⁺ |
| | phenylethyl)-2-methyl-7H-pyrrolo[2,3- | | | | |
| | d]pyrimidin-4-amine | | | | |
| 124 | 5-(2-aminopyridin-4-yl)-2-methyl-N-[2- | C19H19N7 | 345.1702 | 346.1767 | [M − H]⁺ |
| | (pyridin-2-yl)ethyl]-7H-pyrrolo[2,3- | | | | |
| | d]pyrimidin-4-amine | | | | |
| 125 | 4-{4-[(2R,6S)-2,6-dimethylmorpholin-4-yl]- | C18H22N6O | 338.1855 | 339.1931 | [M − H]⁺ |
| | 2-methyl-7H-pyrrolo[2,3-d]pyrimidin-5- | | | | |
| | yl}pyridin-2-amine + | | | | |
| | 4-{4-[(2S,6R)-2,6-dimethylmorpholin-4-yl]- | | | | |
| | 2-methyl-7H-pyrrolo[2,3-d]pyrimidin-5- | | | | |
| | yl}pyridin-2-amine | | | | |
| 126 | 4-{4-[(2R,6S)-2,6-dimethylmorpholin-4-yl]- | C18H23N7O | 353.1964 | 354.2044 | [M − H]⁺ |
| | 2-methyl-7H-pyrrolo[2,3-d]pyrimidin-5- | | | | |
| | yl}pyridine-2,6-diamine + | | | | |
| | 4-{4-[(2S,6R)-2,6-dimethylmorpholin-4-yl]- | | | | |
| | 2-methyl-7H-pyrrolo[2,3-d]pyrimidin-5- | | | | |
| | yl}pyridine-2,6-diamine | | | | |
| 127 | 5-(2-aminopyridin-4-yl)-N-(1,3- | C20H18N6O2 | 374.1491 | 375.1494 | [M − H]⁺ |
| | benzodioxol-4-ylmethyl)-2-methyl-7H- | | | | |
| | pyrrolo[2,3-d]pyrimidin-4-amine | | | | |
| 128 | 4-{4-[(1,3-benzodioxol-4-ylmethyl)amino]- | C20H19N7O2 | 389.1600 | 396.1593 | [M − H]⁺ |
| | 2-methyl-7H-pyrrolo[2,3-d]pyrimidin-5- | | | | |
| | yl}pyridine-2,6-diamine | | | | |
| 129 | 5-(2-aminopyridin-4-yl)-2-methyl-N- | C18H16N6 | 316.1436 | 317.1458 | [M − H]⁺ |
| | phenyl-7H-pyrrolo[2,3-d]pyrimidin-4- | | | | |
| | amine | | | | |
| 130 | 5-(2-aminopyridin-4-yl)-2-methyl-N-[2- | C19H15F3N6 | 384.1310 | 385.1326 | [M − H]⁺ |
| | (trifluoromethyl)phenyl]-7H-pyrrolo[2,3- | | | | |
| | d]pyrimidin-4-amine | | | | |
| 131 | 4-(2-methyl-4-{[2- | C19H16F3N7 | 399.1419 | 400.1434 | [M − H]⁺ |
| | (trifluoromethyl)phenyl]amino}-7H- | | | | |
| | pyrrolo[2,3-d]pyrimidin-5-yl)pyridine-2,6- | | | | |
| | diamine | | | | |
| 132 | 5-(2-aminopyridin-4-yl)-N-(1,3- | C19H16N6O2 | 360.1335 | 361.1332 | [M − H]⁺ |
| | benzodioxol-5-yl)-2-methyl-7H- | | | | |
| | pyrrolo[2,3-d]pyrimidin-4-amine | | | | |
| 133 | 4-[4-(4-methoxypiperidin-1-yl)-2-methyl- | C18H22N6O | 338.1855 | 339.1871 | [M − H]⁺ |
| | 7H-pyrrolo[2,3-d]pyrimidin-5-yl]pyridin-2- | | | | |
| | amine | | | | |
| 134 | 4-[2-methyl-4-(morpholin-4-yl)-7H- | C16H18N6O | 310.1542 | 311.1565 | [M − H]⁺ |
| | pyrrolo[2,3-d]pyrimidin-5-yl]pyridin-2- | | | | |
| | amine | | | | |
| 135 | 4-[4-(5,6-dihydroimidazo[1,2-a]pyrazin- | C18H18N8 | 346.1654 | 347.1663 | [M − H]⁺ |
| | 7(8H)-yl)-2-methyl-7H-pyrrolo[2,3- | | | | |
| | d]pyrimidin-5-yl]pyridin-2-amine | | | | |
| 136 | 4-[4-(7,8-dihydropyrido[3,4-b]pyrazin- | C19H18N8 | 358.1654 | 359.1659 | [M − H]⁺ |
| | 6(5H)-yl)-2-methyl-7H-pyrrolo[2,3- | | | | |
| | d]primidin-5-yl]pyridin-2-amine | | | | |
| 137 | 4-[2-methyl-4-(2-methylmorpholin-4-yl)- | C17H20N6O | 324.1699 | 323.1544 | [M − H]⁻ |
| | 7H-pyrrolo[2,3-d]pyrimidin-5-yl]pyridin-2- | | | | |
| | amine | | | | |
| 138 | 4-[4-(3-methoxypiperidin-1-yl)-2-methyl- | C18H22N6O | 338.1855 | 339.1855 | [M − H]⁺ |
| | 7H-pyrrolo[2,3-d]pyrimidin-5-yl]pyridin-2- | | | | |
| | amine | | | | |
| 139 | 4-[4-(4-methoxypiperidin-1-yl)-2-methyl- | C18H23N7O | 353.1964 | 354.1972 | [M − H]⁺ |
| | 7H-pyrrolo[2,3-d]pyrimidin-5-yl]pyridine- | | | | |
| | 2,6-diamine | | | | |
| 140 | 4-[2-methyl-4-(morpholin-4-yl)-7H- | C16H19N7O | 325.1651 | 326.1724 | [M − H]⁺ |
| | pyrrolo[2,3-d]pyrimidin-5-yl]pyridine-2,6- | | | | |
| | diamine | | | | |
| 141 | 4-[4-(5,6-dihydroimidazo[1,2-a]pyrazin- | C18H19N9 | 361.1763 | 362.1764 | [M − H]⁺ |
| | 7(8H)-yl)-2-methyl-7H-pyrrolo[2,3- | | | | |
| | d]pyrimidin-5-yl]pyridine-2,6-diamine | | | | |
| 142 | 4-[4-(7,8-dihydropyrido[3,4-b]pyrazin- | C19H19N9 | 373.1763 | 374.1760 | [M − H]⁺ |
| | 6(5H)-yl)-2-methyl-7H-pyrrolo[2,3- | | | | |
| | d]pyrimidin-5-yl]pyridine-2,6-diamine | | | | |
| 143 | 4-[2-methyl-4-(2-methylmorpholin-4-yl)- | C17H21N7O | 339.1808 | 340.1845 | [M − H]⁺ |
| | 7H-pyrrolo[2,3-d]pyrimidin-5-yl]pyridine- | | | | |
| | 2,6-diamine | | | | |
| 144 | 4-[4-(3-methoxypiperidin-1-yl)-2-methyl- | C18H23N7O | 353.1964 | 354.1978 | [M − H]⁺ |
| | 7H-pyrrolo[2,3-d]pyrimidin-5-yl]pyridine- | | | | |
| | 2,6-diamine | | | | |
| 145 | N-[2-methoxy-6-(trifluoromethyl)benzyl]-2- | C21H18F3N5O | 413.1463 | 414.1468 | [M − H]⁺ |
| | methyl-5-(pyridin-4-yl)-7H-pyrrolo[2,3- | | | | |
| | d]pyrimidin-4-amine | | | | |
| 146 | N-[2-methoxy-6-(trifluoromethyl)benzyl]-2- | C22H20F3N5O | 427.1620 | 428.1633 | [M − H]⁺ |
| | methyl-5-(2-methylpyridin-4-yl)-7H- | | | | |
| | pyrrolo[2,3-d]pyrimidin-4-amine | | | | |

### General Procedure VII

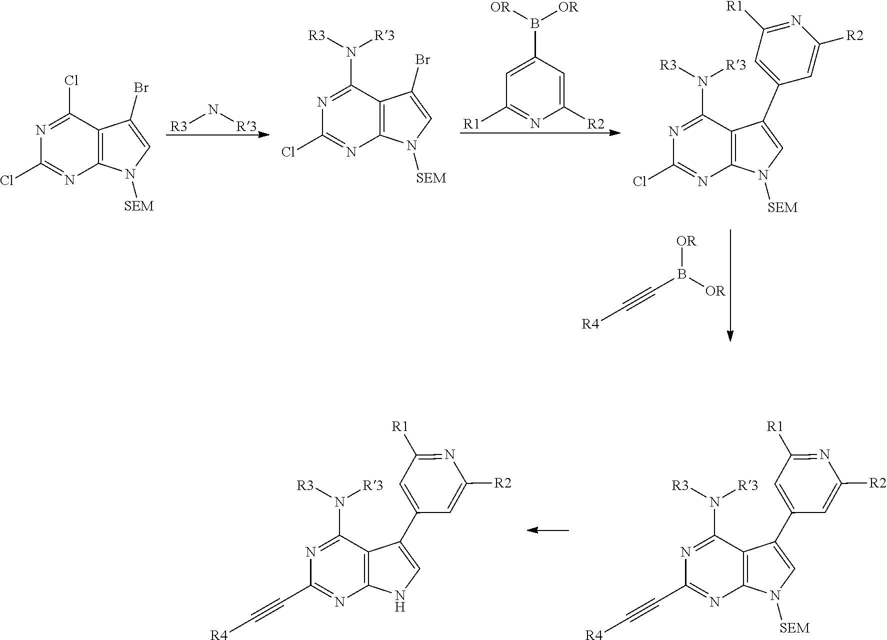

### General Procedure VIII

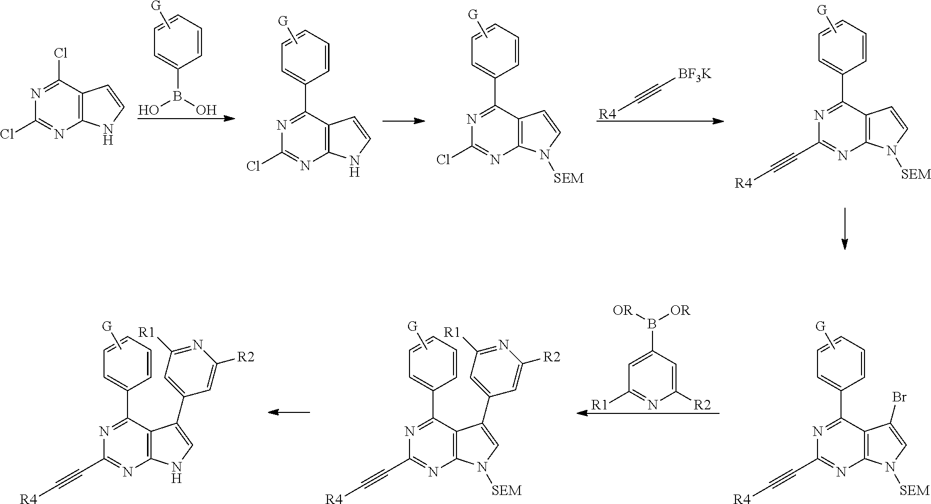

### General Procedure IX

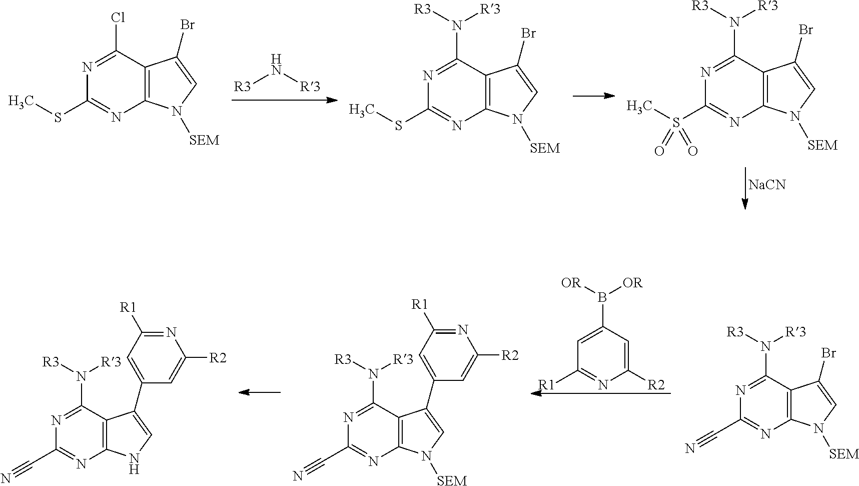

### General Procedure X

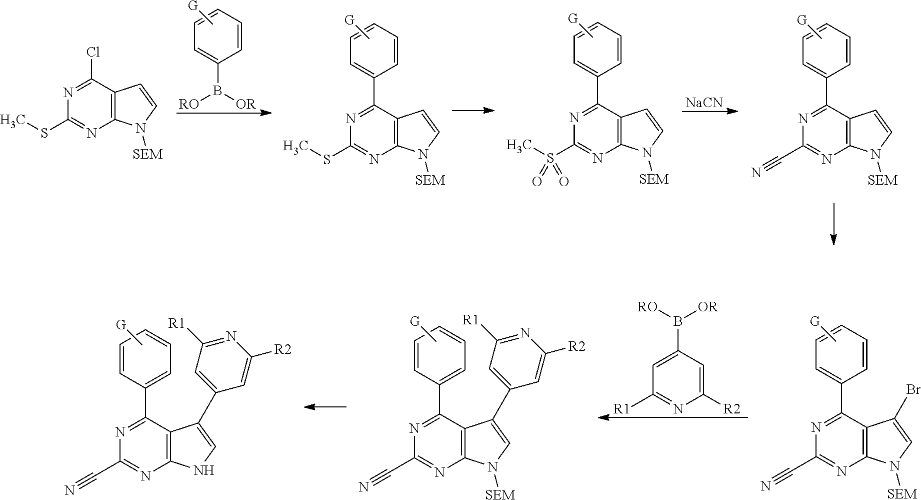

In General Procedures VII, VIII, IX and X:
R₁ and R₂ are as defined in formula (I),
R₃ represents a hydrogen atom, a linear or branched (C₁-C₆)alkyl group, —(C₀-C₆)alkylene-Cy₁, —(C₀-C₆)alkylene-Cy₁-Cy₂, —(C₀-C₆)alkylene-Cy₁-O—(C₁-C₆)alkylene-Cy₂, it being understood that Cy₁ and Cy₂, independently of one another, represent a cycloalkyl group, a heterocycloalkyl group, an aryl or an heteroaryl group,
and R′₃represents a hydrogen atom or a linear or branched (C₁-C₆)alkyl group,
or R₃ and R′₃ form with the nitrogen atom carrying them a heterocycloalkyl or an heteroaryl,
   R₄ represents a hydrogen atom, a linear or branched (C₁-C₆)alkyl group or a cycloalkyl group,
   G represents a group selected from the list of substituents defined in formula (I), it being understood that the phenyl may be substituted by from 1 to 4 independent G groups.

### EXAMPLE 14

### 5-(2-aminopyridin-4-yl)-N-(2,6-difluorobenzyl)-2-ethynyl-7H-pyrrolo[2,3-d]pyrimidin-4-amine

### Step 1: 5-bromo-2-chloro-N-[(2,6-difluorophenyl)methyl]-7-{[2-(trimethylsilyl)ethoxy]methyl}-7H-pyrrolo[2,3-d]pyrimidin-4-amine

Starting from 5-bromo-2,4-dichloro-7-{[2-(trimethylsilyl)ethoxy]methyl}-7H-pyrrolo [2,3-d]pyrimidine (prepared following procedure described in WO2007/104944) (1 g, 2.52 mmol) and (2,6-difluorophenyl)methanamine (2 eq) following procedure described in Preparation 8. The residue was purified via flash chromatography using EtOAc and isohexane as eluent to give the product (1.25 g, 2.48 mmol, 98%) as a clear oil.

LC/MS (method B): RT=3.0 min; m/z=505 [M+H]⁺

### Step 2: tert-butyl N-[4-(2-chloro-4-{[2,6-difluorophenyl)methyl]amino}-7-{[2-(trimethylsilyl)ethoxy]methyl}-7H-pyrrolo[2,3-d]pyrimidin-5-yl)pyridin-2-yl]carbamate

Starting from the compound obtained in Step 1 (1.25 g, 2.48 mmol) and tert-butyl N-[4-(tetramethyl-1,3,2-dioxaborolan-2-yl)pyridin-2-yl]carbamate (1.2 eq) following procedure described in Preparation 3, the desired product (1.063 g, 1.72 mmol, 69%) was obtained as an off-white solid.

¹H NMR (399 MHz, DMSO-d6) δ 9.93 (s, 1H), 8.30 (d, 1H), 7.95 (d, 1H), 7.76 (s, 1H), 7.44 (tt, 1H), 7.19-7.06 (m, 3H), 6.78 (t, 1H), 5.57 (s, 2H), 4.82 (d, 2H), 3.67-3.57 (m, 2H), 1.54 (s, 9H), 0.98-0.84 (m, 2H), 0.00 (s, 9H).

LC/MS (method B): RT=1.71 mm, m/z=617 [M+H]⁺

### Step 3: 4-{2-[2-(tert-butyldimethylsilyl)ethynyl]-4-{[(2,6-difluorophenyl)methyl]amino}-7-{[2-(trimethylsilyl)ethoxy]methyl}-7H-pyrrolo[2,3-d]pyrimidin-5yl}pyridin-2-amine (Preparation 10)

The compound obtained in Step 2 (0.5 g, 0.81 mmol) and tert-butyldimethyl[2-(tetramethyl-1,3,2-dioxaborolan-2-yl)ethynyl]silane (1.2 eq) were dissolved in 1,4-dioxane (10 mL) under N₂. 2M Na₂CO₃ aq. solution (1 mL) and tetrakis(triphenylphosphine)palladium (0.08 mmol) were added and the resulting mixture was degassed under N₂ for 5 minutes. The reaction mixture was heated at 160° C. on a CEM microwave reactor for 1 hour. The reaction mixture was filtered through a plug of celite. The filtrate was diluted with water (10 mL) and EtOAc (50 mL). The organic layer was separated, washed with brine, dried over MgSO₄ and concentrated in vacuo. The residue was purified via flash chromatography using EtOAc and isohexane as eluent to give the product (0.379 g) as a yellow oil. Purity estimated around 50% by LCMS. The compound was used without further purification.

LC/MS (method A): RT=2.84 min; m/z=621 [M+H]⁺

### Step 4: 5-(2-aminopyridin-4-yl)-N-(2,6-difluorobenzyl)-2-ethynyl-7H-pyrrolo[2,3-d]pyrimidin-4-amine

Starting from the compound obtained in Step 3 (0.379 g) following procedure described in Preparation 4, the desired product (13 mg, 0.003 mmol) was obtained as a white solid.

¹H NMR (400 MHz, DMSO-d6) δ 12.19 (s, 1H), 7.97 (d, 1H), 7.54-7.41 (m, 2H), 7.19 (q, 2H), 6.62-6.54 (m, 2H), 6.09 (t, 1H), 6.03 (s, 2H), 4.86 (d, 2H), 4.06 (s, 1H).

LC/MS (method B): RT=0.99 min; m/z=377 [M+H]⁺

### EXAMPLE 153

### 4-[4-(1,3-benzodioxol-5-yl)-2-(cyclopropylethynyl)-7H-pyrrolo[2,3-d]pyrimidin-5-yl]pyridin-2-amine

### Step 1: 4-(1,3-benzodioxol-5-yl)-2-chloro-7H-pyrrolo[2,3-d]pyrimidine

Starting from 2,4-dichloro-7H-pyrrolo[2,3-d]pyrimidine (1 g, 5.32 mmol) and (1,3-benzodioxol-5-yl)boronic acid (1.05 eq) following procedure described in Preparation 3, the desired product (1.45 g, 3.84 mmol) was obtained as a pale yellow solid. Purity estimated around 70% by LCMS. The compound was used without further purification.

LC/MS (method B): RT=1.2 min; m/z=274 [M+H]⁺

### Step 2: 4-(1,3-benzodioxol-5-yl)-2-chloro-7-{[2-(trimethylsilyl)ethoxy]methyl}-7H-pyrrolo[2,3-d]pyrimidine

Starting from the compound obtained in Step 1 (1.45 g, 3.84 mmol) following procedure described in Preparation 1, the desired product (1.005 g, 2.49 mmol, 65%) was obtained as a white solid.

¹H NMR (399 MHz, DMSO-d6) δ 7.92 (d, 1H), 7.85 (dd, 1H), 7.73 (d, 1H), 7.21 (d, 1H), 7.12 (d, 1H), 6.25 (s, 2H), 5.68 (s, 2H), 3.73-3.53 (m, 2H), 0.99-0.83 (m, 2H), 0.00 (s, 9H).

LC/MS (method B): RT=1.57 min; m/z=404 [M+H]⁺

Step 3: 4-(1,3-benzodioxol-5-yl)-2-(cyclopropylethynyl)-7-{[2-(trimethylsilyl)ethoxy]methyl}-7H-pyrrolo[2,3-d]pyrimidine

Starting from the compound obtained in Step 2 (0.45 g, 1.11 mmol) and potassium (2-cyclopropyl-ethyn-1-yl)-trifluoroborate (prepared from Org. Lett., 2010, 12, 3272-3275) (1.4 eq) following procedure described in Preparation 10, the desired product (0.22 g, 0.512 mmol, 46%) was obtained as a red oil.

¹H NMR (399 MHz, DMSO-d6) δ 7.95 (dd, 1H), 7.89-7.77 (m, 1H), 7.73 (dd, 1H), 7.26-7.03 (m, 2H), 6.27-6.18 (m, 2H), 5.71 (s, 2H), 3.74-3.58 (m, 2H), 1.50 (m, 1H), 1.01-0.83 (m, 6H), 0.00 (s, 9H).

LC/MS (method B): RT=1.61 min; m/z=434 [M+H]⁺

### Step 4: 4-(1,3-benzodioxol-5-yl)-5-bromo-2-(cyclopropylethynyl)-7-{[2- (trimethylsilyl)ethoxy]methyl}-7H-pyrrolo[2,3-d]pyrimidine (Preparation 11)

To solution of the compound obtained in Step 3 (0.22 g, 0.512 mmol) in DMF (10 mL) was added NBS (1.05 eq) 0° C. under N₂ and the reaction was allowed to warm to room temperature over 3 hours. The reaction mixture was diluted with water (20 mL) and EtOAc (20 mL). The organic layer was separated, washed with brine, dried over MgSO₄ and concentrated in vacuo. The residue was purified via flash chromatography using EtOAc and isohexane as eluent to give the product (0.147 g, 0.286 mmol, 56%) as a brown oil.

¹H NMR (399 MHz, DMSO-d6) δ 8.13 (s, 1H), 7.32-7.22 (m, 2H), 7.13 (d, 1H), 6.20 (s, 2H), 5.66 (s, 2H), 3.67-3.58 (m, 2H), 1.69 (tt, 1H), 1.04-0.99 (m, 2H), 0.95-0.86 (m, 4H), 0.00 (s, 9H).

LC/MS (method B): RT=1.64 min; m/z=512 [M+H]⁺

### Step 5: tert-butyl N-{4-[4-(1,3-benzodioxol-5-yl)-2-(cyclopropylethynyl)-7-{[2-(trimethylsilyl)ethyl]methyl}-7H-pyrrolo[2,3-d]pyrimidin-5-yl]pyridin-2-yl}carbamate

Starting from the compound obtained in Step 4 (0.110 g, 0.21 mmol) and tert-butyl N-[4-(tetramethyl-1,3,2-dioxaborolan-2-yl)pyridin-2-yl]carbamate (1.1 eq) following procedure described in Preparation 3, the desired product (96 mg, 0.153 mmol, 71%) was obtained as an off-white solid.

LC/MS (method B): RT=1.63 min; m/z=626 [M+H]⁺

### Step 6: 4-[4-(1,3-benzodioxol-5-yl)-2-(cyclopropylethynyl)-7H-pyrrolo[2,3-d]pyrimidin-5-yl]pyridin-2-amine

Starting from the compound obtained in Step 5 (96 mg, 0.153 mmol) following procedure described in Preparation 7, the desired product (34 mg, 0.083 mmol, 54%) was obtained as an off-white solid.

¹H NMR (399 MHz, DMSO-d6) δ 12.51 (s, 1H), 7.83 (s, 1H), 7.61 (d, 1H), 6.96 (d, 1H), 6.88-6.80 (m, 1H), 6.74 (d, 1H), 6.15 (t, 1H), 6.02 (s, 2H), 6.04-5.98 (m, 1H), 5.68 (s, 2H), 1.63 (tt, 1H), 1.07-0.90 (m, 2H), 0.91-0.79 (m, 2H).

LC/MS (method B): RT=0.99 min; m/z=396 [M+H]⁺

### EXAMPLE 157

### 5-(2-aminopyridin-4-yl)-4-[(2,6-difluorobenzyl)amino]-7H-pyrrolo[2,3-d]pyrimidine-2-carbonitrile

Step 1: 5-bromo-N-[(2,6-difluorophenyl)-methyl]-2-(methylsulfanyl)-7-{[2-(trimethylsilyl) ethoxy]methyl}-7H-pyrrolo[2,3-d]pyrimidin-4-amine

Starting from 5-bromo-4-chloro-2-(methylsulfanyl)-7-{[2-(trimethylsilyl)ethoxy]methyl}-7H-pyrrolo[2,3-d]pyrimidine (prepared following procedure described in WO2007/104944) (0.77 g, 1.88 mmol) and 2,6-difluorobenzylamine (3 eq) following procedure described in Preparation 8, the desired product (0.856 g, 1.66 mmol, 88%) was obtained as a pale yellow oil.

¹H NMR (399 MHz, DMSO-d6) δ 7.49 (m, 2H), 7.18 (t, 2H), 6.97 (s, 1H), 5.49 (s, 2H), 4.94 (d, 2H), 3.58 (m, 2H), 2.55 (s, 3H), 0.98-0.87 (m, 2H), 0.00 (s, 9H).

LC/MS (method B): RT=1.7 min; m/z=515 [M+H]⁺

### Step 2: 5-bromo-N-[(2,6-difluorophenyl)methyl]-2-methanesulfonyl-7-{[2-(trimethylsilyl) ethoxy]methyl}-7H-pyrrolo[2,3-d]pyrimidin-4-amine (Preparation 12)

To a solution of the compound obtained in Step 1 (0.856 g. 1.66 mmol) in DCM (20 mL) was added mCBPA (2.5 eq) portion wise at 0° C. under N₂. The reaction mixture was stirred at the same temperature for 1 hour before allowed to warm to room temperature over 2 hours. The reaction mixture was diluted with sat. aq. NaHCO₃ (20 mL) solution and DCM (20 mL). The organic layer was separated, washed with brine, dried over MgSO₄ and concentrated in vacuo to give the product (0.831 g, 1.51 mmol, 92%) as a yellow oil. The compound was used without further purification.

LC/MS (method B): RT=1.53 min; m/z=549 [M+H]⁺

### Step 3: 5-bromo-4-{[(2,6-difluorophenyl)methyl]amino}-7-{[2-(trimethylsilyl) ethoxy]methyl}-7H-pyrrolo[2,3-d]pyrimidine-2-carbonitrile (Preparation 13)

To a solution of the compound obtained in Step 2 (0.660 g, 1.11 mmol) in DMF (15 mL) was added sodium cyanide (2.5 eq) under N₂ at room temperature. The reaction mixture was heated at 90° C. for 2 hours. The reaction mixture was cooled to room temperature, diluted with water (20 mL) and EtOAc (20 mL). The organic layer was separated, washed with brine, dried over MgSO₄ and concentrated in vacuo. The residue was purified via flash chromatography using EtOAc and isohexane as eluent to give the product (0.453 g, 0.916 mmol, 76%) as a clear oil.

¹H NMR (399 MHz, DMSO-d6) δ 7.97 (s, 1H), 7.55-7.41 (m, 2H), 7.19 (t, 2H), 5.58 (s, 2H), 4.93 (d, 2H), 3.63-3.53 (m, 2H), 0.99-0.83 (m, 2H), 0.00 (s, 9H).

LC/MS (method A): RT=2.94 min; m/z=496 [M+H]⁺

### Step 4: tert-butyl N-[4-(2-cyano-4-{[2,6-difluorophenyl)methyl]amino}-7-{[2-(trimethylsilyl)ethoxy]methyl}-7H-pyrrolo[2,3-d]pyrimidin-5-yl)pyridin-2-yl]carbamate

Starting from the compound obtained in Step 3 (0.225 g, 0.46 mmol) an tert-butyl N-[4-(tetramethyl-1,3,2-dioxaborolan-2-yl)pyridin-2-yl]carbamate (1.1 eq) following procedure described in Preparation 3, the desired product (0.135 g, 1.51 mmol, 49%) was obtained as a white solid.

¹H NMR (399 MHz, DMSO-d6) δ 9.96 (s, 1H), 8.32 (d, 1H), 8.03 (s, 1H), 7.97 (d, 1H), 7.45 (t, 1H), 7.19-7.08 (m, 3H), 6.94 (t, 1H), 5.67 (s, 2H), 4.84 (d, 2H), 3.63 (t, 2H), 0.99-0.85 (m, 2H), 0.00 (s, 9H).

LC/MS (method A): RT=2.98 mm; m/z=608 [M+H]⁺

### Step 5: 5-(2-aminopyridin-4-yl)-4-[(2,6-difluorobenzyl)amino]-7H-pyrrolo[2,3-d]pyrimidine-2-carbonitrile

Starting from the compound obtained in Step 4 (0.135 g, 1.51 mmol) following procedure described in Preparation 7, the desired product (17 mg, 0.04 mmol) was obtained as a white solid.

¹H NMR (399 MHz, DMSO-d6) δ 12.56 (s, 1H), 7.92 (d, 1H), 7.64 (s, 1H), 7.40 (tt, 1H), 7.11 (t, 2H), 6.54-6.43 (m, 3H), 5.98 (s, 2H), 4.77 (d, 2H).

LC/MS (method B): RT=1.03 min; m/z=378 [M+H]⁺

### EXAMPLE 158

### 4-(1,3-benzodioxol-5-yl)-5-(2,6-diaminopyridin-4-yl)-7H-pyrrolo[2,3-d]pyrimidine-2-carbonitrile

### Step 1: 4-(1,3-benzodioxol-5-yl)-2-(methylsulfanyl)-7-{[2-(trimethylsilyl)ethoxy]methyl}-7H-pyrrolo[2,3-d]pyrimidine

Starting from 4-chloro-2-(methylsulfanyl)-7-{[2-(trimethylsilyl)ethoxy]methyl}-7H-pyrrolo[2,3-d]pyrimidine (prepared following procedure described in WO2007/104944) (0.411 g, 1.25 mmol) and (1,3-benzodioxol-5-yl)boronic acid (1.1 eq) following procedure described in Preparation 3, the desired product (0.462 g, 1.11 mmol, 89%) was obtained as a pale yellow oil.

¹H NMR (399 MHz, DMSO-d6) δ 7.84 (dd, 1H), 7.78-7.69 (m, 2H), 7.20 (d, 1H), 6.99 (d, 1H), 6.14 (s, 2H), 5.68 (s, 2H), 3.68 (m, 2H), 2.71 (s, 3H), 1.00-0.86 (m, 2H), 0.00 (s, 9H).

LC/MS (method B): RT=1.63 min; m/z=416 [M+H]⁺

### Step 2: 4-(1,3-benzodioxol-5-yl)-2-methanesulfonyl-7-{[2-(trimethylsilyl)ethoxy]methyl}-7H-pyrrolo[2,3-d]pyrimidine

Starting from the compound obtained in Step 1 (0.462 g, 1.11 mmol) following procedure described in Preparation 12, the desired product (0.475 g, 1.06 mmol, 95%) was obtained as a pale orange oil.

¹H NMR (399 MHz, DMSO-d6) δ 8.19 (d, 1H), 8.01-7.92 (m, 2H), 7.86 (d, 1H), 7.29 (d, 1H), 6.27 (s, 2H), 5.81 (s, 2H), 3.73-3.61 (m, 2H), 3.57 (s, 3H), 1.01-0.92 (m, 2H), 0.00 (s, 9H).

LC/MS (method A): RT=2.7 min; m/z=448 [M+H]⁺

### Step 3: 4-(1,3-benzodioxol-5-yl)-7-{[2-(trimethylsilyl)ethoxy]methyl}-7H-pyrrolo[2,3-d]pyrimidine-2-carbonitrile

Starting from the compound obtained in Step 2 (0.260 g, 0.58 mmol) following procedure described in Preparation 13, the desired product (0.200 g, 0.51 mmol, 87%) was obtained as a dark oil.

LC/MS (method A): RT=2.84 min; m/z=395 [M+H]⁺

### Step 4: 4-(1,3-benzodioxol-5-yl)-5-bromo-7-{[2-(trimethylsilyl)ethoxy]methyl}-7H-pyrrolo[2,3-d]pyrimidine-2-carbonitrile

Starting from the compound obtained in Step 3 (0.200 g, 0.51 mmol) following procedure described in Preparation 11, the desired product (0.183 g, 0.386 mmol, 76%) was obtained as a pale yellow solid.

¹H NMR (399 MHz, DMSO-d6) δ 8.45 (s, 1H), 7.0-7.28 (m, 2H), 7.17 (d, 1H), 6.22 (s, 2H), 5.74 (s, 2H), 3.71-3.57 (m, 2H), 0.97-0.89 (m, 2H), 0.00 (s, 9H).

LC/MS (method B): RT=1.59 min; m/z=473 [M+H]⁺

### Step 5: tert-butyl N-[6-(tert-butoxycarbonylamino)-4-(4,4,5,5-tetramethyl-1,3,2-dioxaborolan-2-yl)-2-pyridyl]carbamate (Preparation 14)

(4-bromo-6-tert-butoxycarbonylamino-pyridin-2-yl)carbamicacid tert-butyl ester (prepared following procedure described in J. Org. Chem. 2004, 69, 543-548) (10 g, 25.27 mmol), bis(pinacolato)diboron (1.5 eq), Pd(OAc)₂ (0.05 eq), 1,1′-bis(diphenylphosphino)ferrocene (0.05 eq) and KOAc (3 eq) were dissolved in 1,4-dioxane (160 mL) under N₂ at room temperature. The reaction mixture was stirred at 80° C. overnight under N₂. The reaction mixture was cooled to room temperature, filtered through celite and washed with warm 1,4-dioxane. Solvent was removed in vacuo. The residue was purified via flash chromatography using EtOAc and DCM as eluent to give the product (7.099 g, 16.3 mmol, 63%) as an off-white solid.

¹H NMR (399 MHz, DMSO-d6) δ 8.16 (brs, 2H), 7.92 (s, 2H), 1.54 (s, 18H), 1.34 (s, 12H).

### Step 6: 4-(1,3-benzodioxol-5-yl)-5-(2,6-diaminopyridin-4-yl)-7H-pyrrolo[2,3-d]pyrimidine-2-carbonitrile

The procedure described in Preparation 3 was applied starting from the compound obtained in Step 4 (0.183 g, 0.386 mmol) and the compound obtained in Step 5 (1.1 eq). The crude reaction mixture was concentrated in vacuo and the residue dissolved in DCM (2 mL) and TFA (1.5 mL) following procedure described in Preparation 7, the desired product (8.4 mg, 0.022 mmol, 6%) was obtained as a white solid.

¹H NMR (399 MHz, DMSO-d6) δ 13.07 (s, 1H), 8.02 (s, 1H), 7.09-6.97 (m, 2H), 6.79 (d, 1H), 6.04 (s, 2H), 5.32 (s, 2H), 5.21 (s, 4H).

LC/MS (method B): RT=0.92 min; m/z=372 [M+H]⁺

Examples 147-158 in the following Table 3 were prepared by methods outlined in General Procedure VII-X using appropriate commercially available boronate esters and amines. The compounds of Example 148, 153, 157, 158 are also included.

TABLE 3
HRMS (TOF, ESI) data

| | | | Calcd Exact | | |
|---|---|---|---|---|---|
| Example | Structure | Mol Formula | Mass | Found m/z | Adduct |
| 147 | 5-(2-aminopyridin-4-yl)-2-cyclopropyl-N- | C21H18F2N6 | 392.1561 | 391.1494 | [M − H]⁻ |
| | (2,6-difluorobenzyl)-7H-pyrrolo[2,3- | | | | |
| | d]pyrimidin-4-amine | | | | |
| 148 | 5-(2-aminopyridin-4-yl)-N-(2,6- | C20H14F2N6 | 376.1248 | 375.1193 | [M − H]⁻ |
| | difluorobenzyl)-2-ethynyl-7H-pyrrolo[2,3- | | | | |
| | d]pyrimidin-4-amine | | | | |
| 149 | 5-(2-aminopyridin-4-yl)-N-(2,6- | C21H18F2N6 | 392.1561 | 391.1479 | [M − H]⁻ |
| | difluorobenzyl)-2-(prop-1-en-2-yl)-7H- | | | | |
| | pyrrolo[2,3-d]pyrimidin-4-amine | | | | |
| 150 | 5-(2-aminopyridin-4-yl)-N-(2,6- | C21H20F2N6 | 394.1718 | 393.1650 | [M − H]⁻ |
| | difluorobenzyl)-2-(propan-2-yl)-7H- | | | | |
| | pyrrolo[2,3-d]pyrimidin-4-amine | | | | |
| 151 | 5-(2-aminopyridin-4-yl)-N-(2,6- | C20H16F2N6 | 378.1405 | 377.1342 | [M − H]⁻ |
| | difluorobenzyl)-2-ethenyl-7H-pyrrolo[2,3- | | | | |
| | d]pyrimidin-4-amine | | | | |
| 152 | 5-(2-aminopyridin-4-yl)-2- | C23H18F2N6 | 416.1561 | 417.1618 | [M − H]⁺ |
| | (cyclopropylethynyl)-N-(2,6- | | | | |
| | difluorobenzyl)-7H-pyrrolo[2,3- | | | | |
| | d]pyrimidin-4-amine | | | | |
| 153 | 4-[4-(1,3-benzodioxol-5-yl)-2- | C23H17N5O2 | 395.1382 | 396.1383 | [M − H]⁺ |
| | (cyclopropylethynyl)-7H-pyrrolo[2,3- | | | | |
| | d]pyrimidin-5-yl]pyridin-2-amine | | | | |
| 154 | 4-[4-(1,3-benzodioxol-5-yl)-2- | C23H18N6O2 | 410.1491 | 411.1546 | [M − H]⁺ |
| | (cyclopropylethynyl)-7H-pyrrolo[2,3- | | | | |
| | d]pyrimidin-5-yl]pyridine-2,6-diamine | | | | |
| 155 | 4-[4-(1,3-benzodioxol-5-yl)-2-ethynyl-7H- | C20H14N6O2 | 370.1178 | 371.1182 | [M − H]⁺ |
| | pyrrolo[2,3-d]pyrimidin-5-yl]pyridine-2,6- | | | | |
| | diamine | | | | |
| 156 | 4-[4-(1,3-benzodioxol-5-yl)-2-ethynyl-7H- | C20H13N5O2 | 355.1069 | 356.1108 | [M − H]⁺ |
| | pyrrolo[2,3-d]pyrimidin-5-yl]pyridin-2- | | | | |
| | amine | | | | |
| 157 | 5-(2-aminopyridin-4-yl)-4-[(2,6- | C19H13F2N7 | 377.1200 | 378.1242 | [M − H]⁺ |
| | difluorobenzyl)amino]-7H-pyrrolo[2,3- | | | | |
| | d]pyrimidine-2-carbonitrile | | | | |
| 158 | 4-(1,3-benzodioxol-5-yl)-5-(2,6- | C19H13N7O2 | 371.1131 | 370.1036 | [M − H]⁻ |
| | diaminopyridin-4-yl)-7H-pyrrolo[2,3- | | | | |
| | d]pyrimidine-2-carbonitrile | | | | |

### Example 150 was prepared from Example 149 using method described in Preparation 5.

### General Procedure XI

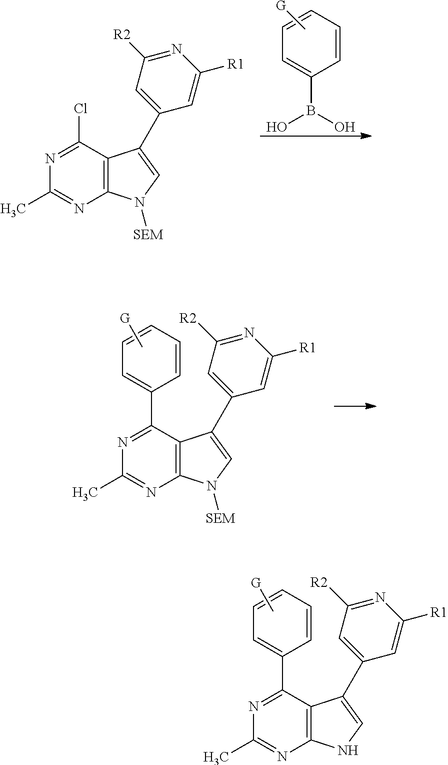

### General Procedure XII

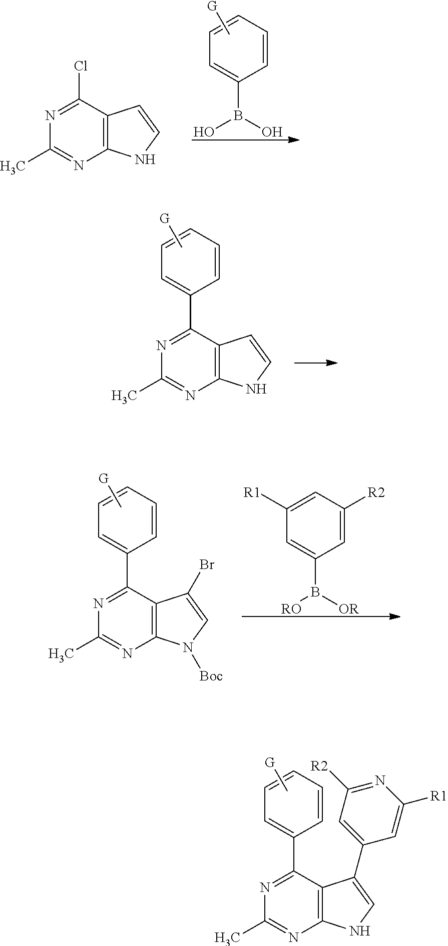

### General Procedure XIII

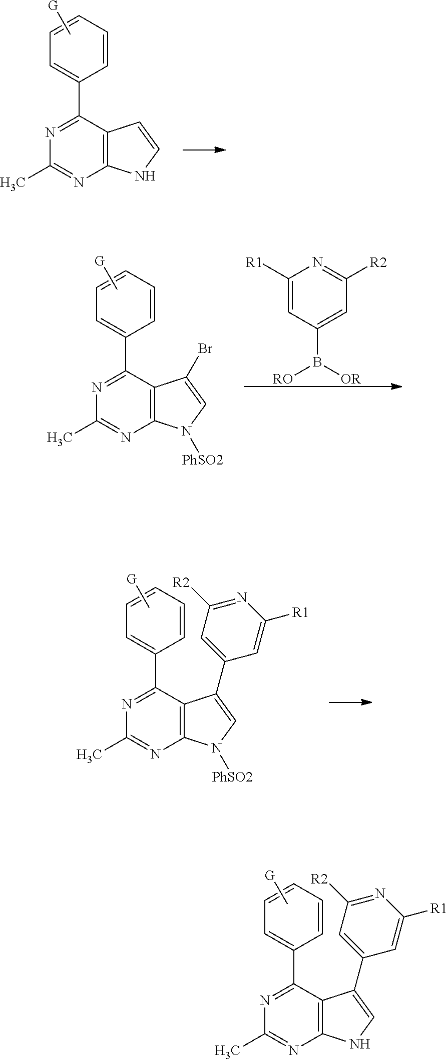

### General Procedure XIV

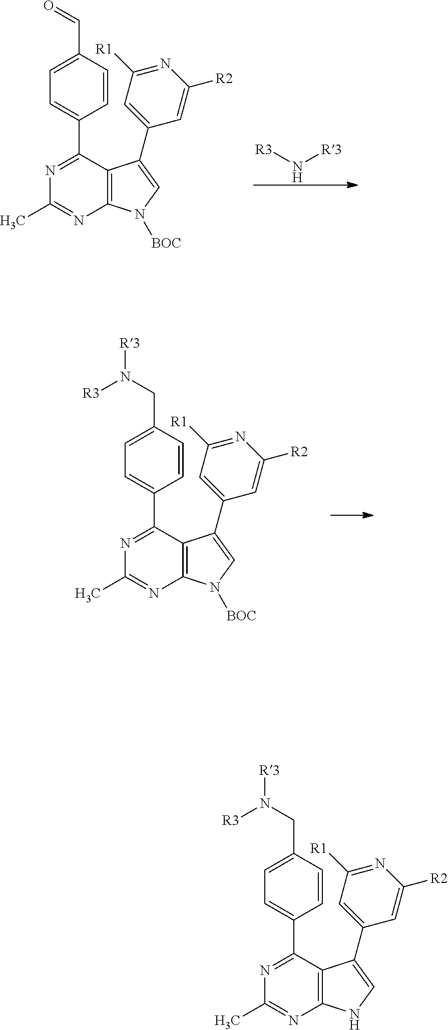

### General Procedure XV

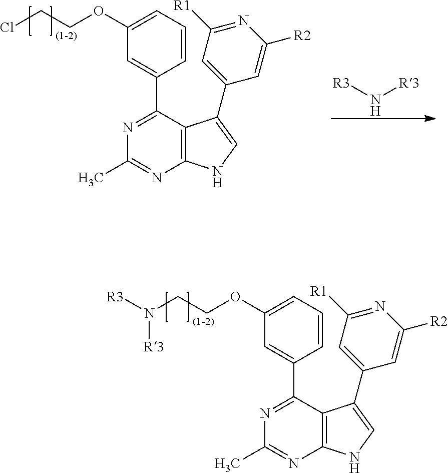

### General Procedure XVI

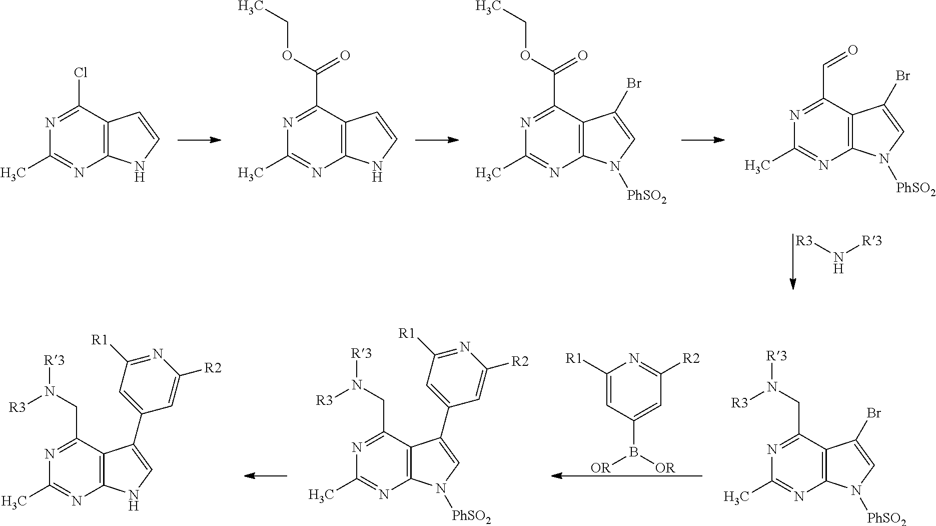

### General Procedure XI to XVII:

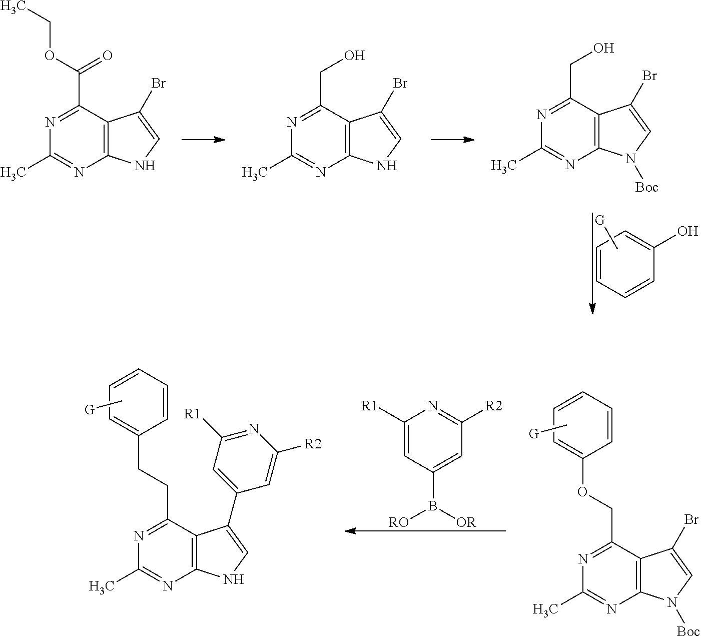

In General Procedures XI to XVII:
R₁and R₂ are as defined in formula (I),
R₃ represents a hydrogen atom, a linear or branched (C₁-C₆)alkyl group, —(C₀-C₆)alkylene-Cy₁, —(C₀-C₆)alkylene-Cy₁Cy₂, —(C₀-C₆)alkylene-Cy₁-O—(C₁-C₆)alkylene-Cy₂, it being understood that Cy₁ and Cy₂, independently of one another, represent a cycloalkyl group, a heterocycloalkyl group, an aryl or an heteroaryl group,
and R′₃ represents a hydrogen atom or a linear or branched (C₁-C₆)alkyl group,
or R₃ and R′₃ form with the nitrogen atom carrying them a heterocycloalkyl or an heteroaryl,
   G represents a group selected from the list of substituents defined in formula (I), it being understood that the phenyl may be substituted by from 1 to 4 independent G groups.

### General Procedure XVIII

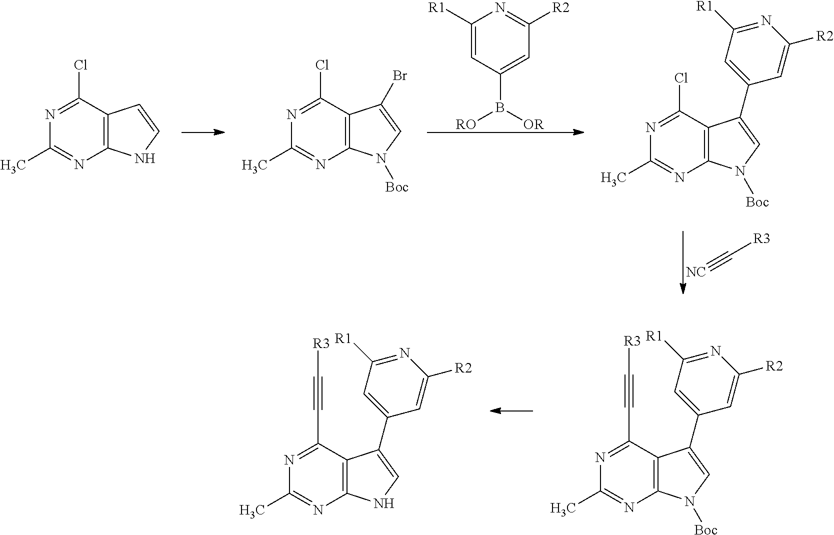

wherein R₃ represents a hydrogen, a cycloalkyl group, a heterocycloalkyl group, an aryl or an heteroaryl group.

### EXAMPLE 162

### 4-[4-(3-fluoro-5-methoxyphenyl)-2-methyl-7H-pyrrolo[2,3-d]pyrimidin-5-yl]pyridin-2-amine

### Step 1: tert-butyl N-{4-[4-3-fluoro-5-methoxyphenyl)-2-methyl-7-{[2-(trimethylsilyl)ethoxy]methyl}-7H-pyrrolo[2,3-d]pyrimidin-5-yl]pyridin-2-yl}carbamate

Starting from tert-butyl N-[4-(4-chloro-2-methyl-7-{[2-(trimethylsilyl)ethoxy]methyl}-7H-pyrrolo[2,3-d]pyrimidin-5-yl)pyridin-2-yl]carbamate (prepared following the procedure described in Example 20, Step 1 using tert-butyl N-[4-(tetramethyl-1,3,2-dioxaborolan-2-yl)pyridin-2-yl]carbamate) (100 mg, 0.2 mmol) and (3-fluoro-5-methoxyphenyl)boronic acid (1.1 eq) following procedure described in Preparation 3, the desired product (104 mg, 0.179 mmol, 88%) was obtained as an off-white solid.

¹H NMR (399 MHz, DMSO-d6) δ 9.69 (s, 1H), 8.15 (s, 1H), 8.06 (d, 1H), 7.52 (s, 1H), 6.94-6.79 (m, 2H), 6.72 (dd, 1H), 6.66 (dd, 1H), 5.77 (s, 2H), 3.70 (dd, 2H), 3.5 (s, 3H), 2.82 (s, 3H), 1.49 (s, 9H), 1.00-0.81 (m, 2H), 0.00 (s, 9H).

LC/MS (method B): RT=1.66 min; m/z=580 [M+H]⁺

### Step 2: 4-[4-(3-fluoro-5-methoxyphenyl)-2-methyl-7H-pyrrolo[2,3-d]pyrimidin-5-yl]pyridin-2-amine (Preparation 15)

To a solution of the compound obtained in Step 1 (104 mg, 0.179 mmol) in DCM (2 mL) was added boron trifluoride diethyl etherate (2 eq) drop wise at 0° C. under N₂ and the reaction mixture was allowed to warm to room temperature over 4 hours. The reaction mixture was diluted with sat. aq. NaHCO₃ (20 mL) solution and DCM (20 mL). The organic layer was separated and concentrated in vacuo. The residue was dissolved in MeCN (2 mL), ammonium hydroxide solution (28% ammonia in water, 2 mL) was added and the mixture stirred at room temperature for 2 hours. The reaction mixture was concentrated in vacuo and the residue was triturated with diethyl ether to give the product (8.7 mg, 0.024 mmol, 14%) as a pale yellow powder.

¹H NMR (399 MHz, DMSO-d6) δ 12.39 (s, 1H), 7.74 (s, 1H), 7.59 (d, 1H), 6.89 (ddd, 1H), 6.81 (dt, 1H), 6.66 (dd, 1H), 6.20-6.14 (m, 1H), 5.99 (dd, 1H), 5.68 (s, 2H), 3.51 (s, 3H), 2.72 (s, 3H).

LC/MS (method B): RT=0.9 min; m/z=350 [M+H]⁺

### EXAMPLE 164

### 4-[4-(2,2-difluoro-1,3-benzodioxol-5-yl)-2-methyl-7H-pyrrolo[2,3-d]pyrimidin-5-yl]pyridin-2-amine

### Step 1: 4 -(2,2-difluoro-1,3-benzodioxol-5-yl)-2-methyl-7H-pyrrolo[2,3-d]pyrimidine (Preparation 16)

4-chloro-2-methyl-7H-pyrrolo[2,3-d]pyrimidine (0.511 g, 3.05 mmol) and (2,2-difluoro-1,3-benzodioxol-5-yl)boronic acid (1.02 eq) were dissolved in THF/water (10:1, 10 mL) under N₂. Cesium carbonate (2 eq) and Pd(dppf)Cl₂ (10% wt) were added and the resulting mixture was degassed under N₂ for 5 minutes. The reaction mixture was heated at 140° C. on a CEM microwave reactor for 1 hour. The mixture was diluted with water (150 mL) and the resulting precipitated was collected by filtration to give the product (0.88 g, 3.04 mmol, 99%) as an off-white solid.

LC/MS (method B): RT=1.27 min; m/z=290 [M+H]⁺

### Step 2: tert-butyl 5-bromo-4-(2,2-difluoro-1,3-benzodioxol-5-yl)-2-methyl-7H-pyrrolo[2,3-d]pyrimidine-7-carboxylate (Preparation 17)

To a solution of the compound obtained in Step 1 (0.88 g, 3.04 mmol) in DMF (15 mL) was added NBS (1.1 eq) portion wise at 0° C. under N₂ and the reaction mixture was allowed to warm to room temperature over 2 hours (reaction monitored by LCMS) Di-tert-buytl dicarbonate (1.2 eq), DMAP (0.01 eq; and trimethylamine (2 eq) were added to the mixture and stirred overnight under N₂ at room temperature. The reaction mixture was diluted with water (50 mL) and EtOAc (50 mL). The organic layer was separated, washed with brine, dried over MgSO₄ and concentrated in vacuo. The residue was purified via flash chromatography using EtOAc and isohexane as eluent to give the product (0.681 g, 1.45 mmol, 48%) as a pale yellow oil.

¹H NMR (399 MHz, DMSO-d6) δ 8.05 (s, 1H), 7.72 (d, 1H), 7.59 (d, 1H), 7.50 (dd, 1H), 2.75 (s, 3H), 1.64 (s, 9H).

LC/MS (method B): RT=1.6 min; m/z=470 [M+H]⁺

Step 3: 4-[4-(2,2-difluoro-1,3-benzodioxol-5-yl)-2-methyl-7H-pyrrolo[2,3-d]pyrimidin-5 yl]pyridin-2-amine (Preparation 18)

The compound obtained in Step 2 (0.681 g, 1.45 mmol) and tert-butyl N-[4-(tetramethyl-1,3,2-dioxaborolan-2-yl)pyridin-2-yl]carbamate (1.1 eq) were dissolved in THF/water (3:1, 20 ml) under N₂. Potassium carbonate (3 eq) and Pd(dtbpf)Cl₂ (10% wt) were added and the resulting mixture was degassed under N₂ for 5 minutes. The reaction mixture was heated at 65° C. overnight under N₂, cooled to room temperature and diluted with water (10 mL) and DCM (50 mL). The organic layer was separated, washed with brine, dried over MgSO₄ and concentrated in vacuo. The residue was purified via flash chromatography using EtOAc and isohexane as eluent to give the desired coupled compound. The compound was dissolved in 2 M HCl solution in MeOH (4 mL) and heated at 90° C. on a CEM microwave reactor for 1 hour. The reaction mixture was concentrated in vacuo and diluted with 10% IPA in DCM (20 ml), washed with sat. aq. NaHCO₃, dried over MgSO₄ and concentrated in vacuo. The residue was purified via flash chromatography using MeOH and DCM as eluent to give, after trituration with diethyl ether, the product (99 mg, 0.26 mmol, 26%) as a white solid.

¹H NMR (399 MHz, DMSO-d6) δ 12.39 (s, 1H), 7.74 (s, 1H), 7.58 (d, 1H), 7.36 (d, 1H), 7.27 (d, 1H), 7.19 (dd, 1H), 6.07 (t, 1H), 6.00 (dd, 1H), 5.68 (s, 2H), 2.72 (s, 3H).

LC/MS (method B): RT=0.96 min; m/z=382 [M+H]⁺

### EXAMPLE 168

### 4-{2-methyl-4-[3-(trifluoromethyl)phenyl]-7H-pyrrolo[2,3-d]pyrimidin-5-yl}pyridin-2-amine

Step 1: 7-(benzenesulfonyl)-5-bromo-2-methyl-4-[3-(trifluoromethyl)phenyl]-7H-pyrrolo[2,3-d]pyrimidine (Preparation 19)

To a solution of 2-methyl-4-[3-(trifluoromethyl)phenyl]-7H-pyrrolo[2,3-d]pyrimidine (prepared following the procedure described in Example 164, Step 1 using 3-(trifluoromethyl)phenyl]boronic acid) (186 mg, 0.67 mmol) in DMF (5 mL) was added NBS (1.1 eq) at 0° C. under N₂ and the reaction was allowed to warm to room temperature over 2 hours. The reaction mixture was cooled to 0° C., NaH (60% in mineral oil, 1.4 eq) was added and stirred for 5 minutes before adding benzenesulfonyl chloride (1.1 eq) under N₂. The reaction mixture was allowed to warm to room temperature overnight, diluted with water (20 mL) and EtOAc (20 mL). The organic layer was separated, washed with brine, dried over MgSO₄ and concentrated in vacuo. The residue was purified via flash chromatography using EtOAc and isohexane as eluent to give the product (201 mg) as a brown oil. Purity estimated around 70% by LCMS. The compound was used without further purification.

LC/MS (method B): RT=1.57 min; m/z=496 [M+H]⁺

### Step 2: 4-[7-(benzenesulfonyl)-2-methyl-4-[3-(trifluoromethyl)phenyl]-7H-pyrrolo[2,3-d]pyrimidin-5-yl]pyridin-2-amine

Starting from the compound obtained in Step 1 (201 mg) and tert-butyl N-[4-(tetramethyl-1,3,2-dioxaborolan-2-yl)pyridin-2-yl]carbamate (1.1 eq) following procedure described in Preparation 3, the desired product (106 mg, 0.177 mmol, 26% over two steps) was obtained as a yellow oil.

LC/MS (method B): RT=1.22 min; m/z=510 [M+H]⁺

Step 3: 4-{2-methyl-4-[3-(trifluoromethyl)phenyl]-7H-pyrrolo[2,3-d]pyrimidin-5-yl}pyridin-2-amine (Preparation 20)

To a solution of the compound obtained in Step 2 (106 mg, 0.177 mmol) in MeOH (5 mL) was added K₂CO₃ (5 eq) and the resulting suspension was stirred at room temperature overnight. The suspension was filtered, concentrated in vacuo and the residue was purified via flash chromatography using MeOH and DCM as eluent to give the product (10 mg, 0.027 mmol, 15%) as a white solid.

¹H NMR (399 MHz, DMSO-d6) δ 12.42 (s, 1H), 7.87-7.79 (m, 1H), 7.74 (d, 2H), 7.56 (s, 2H), 7.61-7.47 (m, 1H), 6.17-6.11 (m, 1H), 5.90 (dd, 1H), 5.64 (s, 2H), 2.74 (s, 3H).

LC/MS (method B): RT=0.94 min; m/z=370 [M+H]⁺

### EXAMPLE 169

### 4-(2-methyl-4-{4-[(4-methylpiperazin-1-yl)methyl]}-7H-pyrrolo[2,3-d]pyrimidin-2-amine

Step 1: tert-butyl 5-(2-{[(tert-butoxy)carbonyl]amino}pyridin-4-yl)-2-methyl-4-{4-[(4-methylpiperazin-1-yl)methyl]phenyl}-7H-pyrrolo[2,3-d]pyrimidine-7-carboxylate (Preparation 21)

To a solution of tert-butyl 5-(2-{[(tert-butoxy)carbonyl]amino}pyridin-4-yl)-4-(4-formylphenyl)-2-methyl-7H-pyrrolo[2,3-d]pyrimidine-7-carboxylate (prepared following the procedure described in Example 164 using (4-formylphenyl)boronic acid) (200 mg, 0.38 mmol) in MeOH (5 mL) was added 1-methylpiperazine (2 eq) followed by sodium cyanoborohydride (1.5 eq) at room temperature under N₂. The reaction mixture was stirred overnight. Then, it was diluted with sat aq. NaHCO₃ solution (10 mL) and DCM (10 mL). The organic layer was separated, washed with brine, dried over MgSO₄ and concentrated in vacuo. The residue was purified via flash chromatography using MeOH and DCM as eluent to give the product (86 mg, 0.14 mmol, 37%) as a white solid.

¹H NMR (399 MHz, DMSO-d6) δ 9.67 (s, 1H), 8.02-7.93 (m, 2H), 7.34 (s, 1H), 7.23 (d, 2H), 7.06 (d, 2H), 6.75 (dd, 1H), 3.44 (s, 2H), 2.78 (s, 3H), 2.5-2.2 (m, 8H), 2.18 (s, 3H), 1.67 (s, 9H), 1.44 (s, 9H).

LC/MS (method B): RT=1.26 min; m/z=614 [M+H]⁺

Step 2: 4-(2-methyl-4-{4-[(4-methylpiperazin-1-yl)methyl]phenyl}-7H-pyrrolo[2,3-d]pyrimidin-5-yl)pyridin-2-amine (Preparation 22)

The compound obtained in Step 1 (86 mg, 0.14 mmol) was dissolved in 2 M HCl in MeOH solution (4 mL) and heated at 80° C. on a CEM microwave reactor for 1 hour. The mixture was concentrated in vacuo and the residue was triturated with diethyl ether to give the product (58 mg, 0.119 mmol) as an HCl salt.

¹H NMR (399 MHz, DMSO-d6) δ 13.71 (brs, 1H), 13.23 (brs, 1H), 11.91 (brs, 1H), 8.25 (d, 1H), 7.77 (m, 4H), 7.56 (d, 2H), 6.48 (dd, 1H), 6.39 (d, 1H), 4.7-3.2 (m, 13H), 2.81 (s, 3H).

LC/MS (method B): RT=0.7 min; m/z=414 [M+H]⁺

### EXAMPLE 174

### 4-(2-methyl-4-{3-[3-(morpholin-4-yl)propoxy]phenyl}-7H-pyrrolo[2,3-d]pyrimidin-5-yl)pyridin-2-amine (Preparation 23)

To a solution of 4-{4-[3-(3-chloropropoxy)phenyl]-2-methyl-7H-pyrrolo[2,3-d]pyrimidin-5-yl}pyridin-2-amine (prepared following the procedure described in Example 168 using 2-[3-(3-chloropropoxy)phenyl]-4,4,5,5-tetramethyl-1,3,2-dioxaborolane (US2007/0004675)) (50 mg, 0.13 mmol) in MeCN (2 mL) was added NaI (4 eq), K₂CO₃ (6 eq) and morpholine (4 eq). The reaction mixture was heated at 150° C. on a CEM microwave reactor for 30 minutes. The reaction mixture was diluted with 10% MeOH in DCM (5 ml), filtered through a phase separator column and concentrated in vacuo. The residue was purified via flash chromatography using MeOH and DCM as eluent to give, after trituration with MeCN, the product (30 mg, 0.067 mmol, 53%) as an off-white solid.

¹H NMR (399 MHz, DMSO-d6) δ 12.33 (s, 1H), 7.70 (s, 1H), 7.51 (d, 1H), 7.21 (t, 1H), 7.12 (dt, 1H), 6.95-6.87 (m, 1H), 6.85-6.79 (m, 1H), 6.19 (d, 1H), 5.91 (dd, 1H), 5.63 (s, 2H), 3.67 (t, 2H), 3.55 (t, 4H), 2.71 (s, 3H), 2.36 (s, 6H), 1.81-1.72 (m, 2H).

LC/MS (method B): RT=0.617 min; m/z=445 [M+H]⁺

### EXAMPLE 178

### 4-[4-(2,3-dihydro-1H-indol-1-ylmethyl)-2-methyl-7H-pyrrolo[2,3-d]pyrimidin-5-yl]pyridin-2-amine

### Step 1: ethyl 2-methyl-7H-pyrrolo[2,3-d]pyrimidine-4-carboxylate (Preparation 24)

4-chloro-2-methyl-7H-pyrrolo[2,3-d]pyrimidine (4 g, 23.87 mmol), sodium acetate (2 eq), Pd(OAc)₂ (0.07 eq) and 1,1′-bis(diphenylphosphino)ferrocene (0.07 eq) in ethanol (140 mL) were combined in a Parr reaction bottle under N₂. The system was purged three times with carbon monoxide and pressurized to 28 psi. The reactor was warmed to 70° C. and shaken overnight in a Parr shaker hydrogenator apparatus. The reactor was cooled to room temperature, carbon monoxide removed by vacuum and the reaction mixture was filtered through a plug of celite. The filtrate was concentrated in vacuo and the residue was triturated with water and diethyl ether to give the product (3.811 g, 18.58 mmol, 78%) as a pale brown solid.

¹H NMR (399 MHz, DMSO-d6) δ 12.24 (s, 1H), 7.69 (d, 1H), 6.81 (d, 1H), 4.43 (q, 2H), 2.71 (s, 3H), 1.39 (t, 3H).

LC/MS (method B): RT=0.92 min; m/z=206 [M+H]⁺

### Step 2: ethyl 7-(benzenesulfonyl)-5-bromo-2-methyl-7H-pyrrolo[2,3-d]pyrimidine-4-carboxylate

Starting from the compound obtained in Step 1 (1.83 g, 3.8 mmol) following procedure described in Preparation 19, the desired product (1.63 g, 3.8 mmol, 60%) was obtained as a white solid.

¹H NMR (399 MHz, DMSO-d6) δ 8.36 (s, 1H), 8.25-8.17 (m, 2H), 7.85-7.74 (m, 1H), 7.74-7.64 (m, 2H), 4.44 (q, 2H), 2.75 (s, 3H), 1.34 (t, 3H).

LC/MS (method B): RT=1.41 min; m/z=423 [M+H]⁺

Step 3: 7-(benzenesulfonyl)-5-bromo-2-methyl-7H-pyrrolo[2,3-d]pyrimidine-4-carbaldehyde (Preparation 25)

To a solution of the compound obtained in Step 2 (0.5 g, 1.18 mmol) in THF (13 mL) was added DIBAL (1M in THF solution. 3 eq) at −78° C. under N₂. The reaction mixture was stirred at the same temperature for 1 hour and allowed to warm to room temperature over 2 hours. Cooled to −78° C., the mixture was quenched with water (1 mL) and 2N NaOH solution (0.5 mL) and allowed to warm to room temperature. MgSO₄ was added to the mixture, filtered through a plug of celite and concentrated in vacuo to give the product (1.2 g, >100%). The compound was used without further purification.

LC/MS (method B): RT=1.31 min; m/z=413, [M+H]⁺ not found

### Step 4: 1-{[7-(benzenesulfonyl)-5-bromo-2-methyl-7H-pyrrolo[2,3-d]pyrimidin-4-yl]methyl}-2,3-dihydro-1H-indole

Starting from the compound obtained in Step 3 (1.2 g) and indoline (1.2 eq) following procedure described in Preparation 21, the desired product (0.193 g, 0.399 mmol, 34% over two steps) was obtained as a white solid.

LC/MS (method B): RT=1.57 mm; m/z=482 [M+H]⁺

### Step 5: 4-[7-(benzenesulfonyl)-4-(2,3-dihydro-1H-indol-1-ylmethyl)-2-methyl-7H-pyrrolo [2,3-d]pyrimidin-5-yl]pyridin-2-amine

Starting from the compound obtained in Step 4 (0.193 g, 0.399 mmol) and tert-butyl N-[4-(tetramethyl-1,3,2-dioxaborolan-2-yl)pyridin-2-yl]carbamate (1.1 eq) following procedure described in Preparation 3, the desired product (0.133 g, 0.267 mmol, 67%) was obtained as a white solid.

¹H NMR (399 MHz, DMSO-d6) δ 8.28-8.20 (m, 2H), 7.96-7.88 (m, 2H), 7.83-7.74 (m, 1H), 7.75-7.64 (m, 2H), 6.93 (dd, 1H), 6.79 (td, 1H), 6.70 (dd, 1H), 6.56 (dd, 1H), 6.50 (td, 1H), 6.09-5.99 (m, 3H), 4.36 (s, 2H), 3.03 (t, 2H), 2.69 (d 5H).

LC/MS (method B): RT=1.16 min; m/z=497 [M+H]⁺

Step 6: 4-[4-(2,3-dihydro-1H-indol-1-ylmethyl)-2-methyl-7H-pyrrolo[2,3-d]pyrimidin-5-yl]pyridin-2-amine

Starting from the compound obtained in Step 5 (0.133 g, 0.267 mmol) following procedure described in Preparation 20, the product (41 mg, 0.114 mmol, 43%) was obtained as an off-white solid.

¹H NMR (399 MHz, DMSO-d6) δ 12.18 (d, 1H), 7.89 (d, 1H), 7.54 (d, 1H), 6.94 (dd, 1H), 6.81 (td, 1H), 6.65 (dd, 1H), 6.57-6.45 (m, 2H), 6.17 (d, 1H), 5.92 (s, 2H), 4.45 (s, 2H), 3.10 (t, 2H), 2.71 (t, 2H), 2.64 (s, 3H).

LC/MS (method B): RT=0.89 min; m/z=357 [M+H]⁺

### EXAMPLE 193

### 4-(2-methyl-4-{[2-(trifluoromethyl)phenoxy]methyl}-7H-pyrrolo[2,3-d]pyrimidin-5-yl)pyridin-2-amine

### Step 1: {5-bromo-2-methyl-7H-pyrrolo[2,3-d]pyrimidin4-yl}methanol (Preparation 26)

To a solution of ethyl 5-bromo-2-methyl-7H-pyrrolo[2,3-d]pyrimidine-4-carboxylate (prepared following the procedure described in Example 153, Step 4 starting from ethyl 2-methyl-7H-pyrrolo[2,3-d]pyrimidine-4-carboxylate (Preparation 24)) (0.500 g, 1.76 mmol) in THF (10 mL) was added LiBH₄ (2 eq) portion wise at 0° C. under N₂. The reaction mixture was allowed to warm to room temperature overnight. The reaction mixture was diluted with sat aq. NaHCO₃ (10 mL) solution and EtOAc (10 mL). The organic layer was separated, washed with brine, dried over MgSO₄ and concentrated in vacuo. The residue was purified via flash chromatography using MeOH and DCM as eluent to give the product (0.237 g, 0.98 mmol, 56%) as a white solid.

¹H NMR (399 MHz, DMSO-d6) δ 12.29 (s, 1H), 7.67 (s, 1H), 5.23 (t, 1H), 4.96 (d, 2H), 2.65 (s, 3H).

LC/MS (method B): RT=0.51 min; m/z=243 [M+H]⁺

### Step 2: tert-butyl 5-bromo-4-(hydroxymethyl)-2-methyl-7H-pyrrolo[2,3-d]pyrimidine-7-carboxylate

To a solution of the compound obtained in Step 1 (0.237 g, 0.98 mmol) was added di-tert-butyl dicarbonate (1.2 eq), DMAP (0.01 eq) and trimethylamine (2 eq) following procedure described in Preparation 17. The desired product (0.345 g, >100%) was obtained as a white solid. Purity estimated around 70% by LC-MS. The compound was used without further purification.

LC/MS (method B): RT=1.23 min; m/z=342 [M+H]⁺

### Step 3: tert-butyl 5-bromo-2-methyl-4-{[2-(trifluoromethyl)phenoxy]methyl}-7H-pyrrolo[2,3-d]pyrimidin-7-carboxylate

Starting from the compound obtained in Step 2 (0.345 g) and 2-(trifluoromethyl)phenol (1.1 eq) following procedure described in Preparation 6, the desired product (0.63 g, >100%) was obtained as a yellow oil. Purity estimated around 45% by LC-MS. The compound was used without further purification.

LC/MS (method B): RT=1.58 min; m/z=485 [M+H]⁺

### Step 4: tert-butyl 5-(2-{[(tert-butoxy)carbonyl]amino}pyridin-4-yl)-2-methyl-4-{[2-4trifluoromethyl)phenoxy]methyl}-7H-pyrrolo[2,3-d]pyrimidin-7-carboxylate

Starting from the compound obtained in Step 3 (0.63 g) and tert-butyl N-[4-(tetramethyl-1,3,2-dioxaborolan-2-yl)pyridin-2-yl]carbamate (1.1 eq) following procedure described in Preparation 18, the desired product (62 mg, 0.155 mmol) was obtained as a white solid.

¹H NMR (399 MHz, DMSO-d6) δ 12.34 (s, 1H), 7.67 (s, 1H), 7.62-7.53 (m, 3H), 7.22 (d, 1H), 7.09 (t, 1H), 6.63 (dd, 1H), 6.51 (t, 1H), 5.75 (d, 2H), 5.31 (s, 2H), 2.66 (s, 3H).

LC/MS (method B): RT=0.99 min; m/z=400 [M+H]⁺

### EXAMPLE 198

### 4-[4-(cyclopropylethynyl)-2-methyl-7H-pyrrolo[2,3-d]pyrimidin-5-yl]pyridin-2-amine

### Step 1: tert-butyl 5-bromo-4-chloro-2-methyl-7H-pyrrolo[2,3-d]pyrimidine-7-carboxylae

Starting from 4-chloro-2-methyl-7H-pyrrolo[2,3-d]pyrimidine (10.53 g, 59.67 mmol) following procedure described in Preparation 17, the product (14.43 g, 41.63 mmol, 93%) was obtained as a white solid.

¹H NMR (399 MHz, DMSO-d6) δ 8.11 (s, 1H), 2.69 (s, 3H), 1.62 (s, 9H).

Step 2: tert-butyl 5-(2-{[(tert-butoxy)carbonyl]amino}pyridin-4-yl)-4-chloro-2-methyl-7H-pyrrolo[2,3-d]pyrimidin-7-carboxylate

Starting from the compound obtained in Step 1 (1 g, 2.89 mmol) and tert-butyl N-[4-(tetramethyl-1,3,2-dioxaborolan-2-yl)pyridin-2-yl]carbamate (1.1 eq) following procedure described in Preparation 3, the desired product (1.059 g, 2.3 mmol, 80%) was obtained as a pale yellow solid.

¹H NMR (399 MHz, DMSO-d6) δ 9.89 (s, 1H), 8.31 (dd, 1H), 8.02 (s, 1H), 7.97 (t, 1H), 7.20 (dd, 1H), 2.71 (s, 3H), 1.64 (s, 9H), 1.48 (s, 9H).

LC/MS (method B): RT=1.49 min; m/z=460 [M+H]⁺

Step 3: tert-butyl 5-(2-{[(tert-butyoxy)carbonyl]amino}pyridin-4-yl)-4-(cyclopropyl ethynyl)-2-methyl-7H-pyrrolo[2,3-d]pyrimidine-7-carboxylate (Preparation 27)

To a solution of the compound obtained in Step 2 (100 mg, 0.22 mmol) in Et₃N (4 ml) and THF (1 mL) was added ethynylcyclopropane (3 eq) and CuI (0.3 eq) at room temperature. The solution was purged with N₂ for 5 minutes before adding Pd(PPh₃)₂Cl₂ (0.3 eq) and the reaction mixture was stirred at 80° C. for 5 hours on a CEM microwave reactor. The reaction mixture cooled to room temperature and concentrated in vacuo. The residue was purified via flash chromatography using MeOH and DCM as ducat to give the product (70 mg, 0.143 mmol, 66%) as a white solid.

LC/MS (method B): RT=1.51 min; m/z=490 [M+H]⁺

### Step 4: 4-[4-(cyclopropylethynyl)-2-methyl-7H-pyrrolo[2,3-d]pyrimidin-5-yl]pyridine-2-amine

Starting from the compound obtained in Step 3 (70 mg, 0.14.3 mmol) following procedure described in Preparation 7, the desired product (32 mg, 0.11 mmol, 77%) was obtained as a white solid.

¹H NMR (399 MHz, DMSO-d6) δ 12.27 (s, 1H), 7.91 (d, 1H), 7.67 (d, 1H), 6.67 (dd, 1H), 6.59 (t, 1H), 5.91 (s, 2H), 2.60 (s, 3H), 1.50 (tt, 1H), 0.85 (m, 2H), 0.66 (m, 2H).

LC/MS (method B): RT=0.76 min; m/z=290 [M+H]⁺

Examples 159-204 in the following Table 4 were prepared by methods outlined in General Procedure XI-XVIII using appropriate commercially available boronate ester, alcohol, amines and ethynyl. The compounds of Example 162, 164, 168, 169, 174, 178, 193, 198 are also included.

TABLE 4
HRMS (TOF, ESI) data

| | | | Calcd Exact | | |
|---|---|---|---|---|---|
| Example | Structure | Mol Formula | Mass | Found m/z | Adduct |
| 159 | 4-{2-methyl-4-[(E)-2-phenylethenyl]-7H- | C20H17N5 | 327.1484 | 328.1564 | [M − H]⁺ |
| | pyrrolo[2,3-d]pyrimidin-5-yl}pyridin-2- | | | | |
| | amine | | | | |
| 160 | 4-[2-methyl-4-(2-phenylethyl)-7H- | C20H19N5 | 329.1640 | 328.1574 | [M − H]⁻ |
| | pyrrolo[2,3-d]pyrimidin-5-yl]pyridin-2-amine | | | | |
| 161 | 4-[4-(1H-indol-2-yl)-2-methyl-7H- | C20H16N6 | 340.1436 | 341.1519 | [M − H]⁺ |
| | pyrrolo[2,3-d]pyrimidin-5-yl]pyridin-2-amine | | | | |
| 162 | 4-[4-(3-fluoro-5-methoxyphenyl)-2-methyl- | C19H16FN5O | 349.1339 | 348.1269 | [M − H]⁻ |
| | 7H-pyrrolo[2,3-d]pyrimidin-5-yl]pyridin-2- | | | | |
| | amine | | | | |
| 163 | 4-(2-methyl-4-phenyl-7H-pyrrolo[2,3- | C18H15N5 | 301.1327 | 302.1396 | [M − H]⁺ |
| | d]pyrimidin-5-yl)pyridin-2-amine | | | | |
| 164 | 4-[4-(2,2-difluoro-1,3-benzodioxol-5-yl)-2- | C19H13F2N5O2 | 381.1037 | 380.0972 | [M − H]⁻ |
| | methyl-7H-pyrrolo[2,3-d]pyrimidin-5- | | | | |
| | yl]pyridin-2-amine | | | | |
| 165 | 4-{2-methyl-4-[4-(pyrrolidin-1-ylmethyl)phenyl]- | C23H24N6 | 384.2062 | 385.2135 | [M − H]⁺ |
| | 7H-pyrrolo[2,3-d]pyrimidin-5- | | | | |
| | yl}pyridin-2-amine | | | | |
| 166 | 4-{4-[(2,6-difluorophenoxy)methyl]-2- | C19H15F2N5O | 367.1245 | 366.1172 | [M − H]⁻ |
| | methyl-7H-pyrrolo[2,3-d]pyrimidin-5- | | | | |
| | yl}pyridin-2-amine | | | | |
| 167 | 4-[4-(3-methoxyphenyl)-2-methyl-7H- | C19H17N5O | 331.1433 | 330.1369 | [M − H]⁻ |
| | pyrrolo[2,3-d]pyrimidin-5-yl]pyridin-2-amine | | | | |
| 168 | 4-{2-methyl-4-[3-(trifluoromethyl)phenyl]- | C19H14F3N5 | 369.1201 | 368.1140 | [M − H]⁻ |
| | 7H-pyrrolo[2,3-d]pyrimidin-5-yl}pyridin-2- | | | | |
| | amine | | | | |
| 169 | 4-(2-methyl-4-{4-[(4-methylpiperazin-1- | C24H27N7 | 413.2328 | 412.2268 | [M − H]⁻ |
| | yl)methyl]phenyl}-7H-pyrrolo[2,3- | | | | |
| | d]pyrimidin-5-yl)pyridin-2-amine | | | | |
| 170 | 4-[4-(5-fluoropyridin-3-yl)-2-methyl-7H- | C17H13FN6 | 320.1186 | 319.1112 | [M − H]⁻ |
| | pyrrolo[2,3-d]pyrimidin-5-yl]pyridin-2-amine | | | | |
| 171 | 4-{2-methyl-4-[3-(pyrrolidin-1-yl)phenyl]- | C22H22N6 | 370.1906 | 369.1839 | [M − H]⁻ |
| | 7H-pyrrolo[2,3-d]pyrimidin-5-yl}pyridin-2- | | | | |
| | amine | | | | |
| 172 | 4-[4-(4-ethoxyphenyl)-2-methyl-7H- | C20H19N5O | 345.1590 | 346.1656 | [M + H]⁺ |
| | pyrrolo[2,3-d]pyrimidin-5-yl]pyridin-2-amine | | | | |
| 173 | 4-[4-(2,3-dihydro-1,4-benzodioxin-6-yl)-2- | C20H17N5O2 | 359.1382 | 360.1440 | [M + H]⁺ |
| | methyl-7H-pyrrolo[2,3-d]pyrimidin-5- | | | | |
| | yl]pyridin-2-amine | | | | |
| 174 | 4-(2-methyl-4-{3-[3-(morpholin-4- | C25H28N6O2 | 444.2274 | 445.2250 | [M + H]⁺ |
| | yl)propoxy]phenyl}-7H-pyrrolo[2,3- | | | | |
| | d]pyrimidin-5-yl)pyridin-2-amine | | | | |
| 175 | 4-[5-(2-aminopyridin-4-yl)-2-methyl-7H- | C19H13FN6 | 344.1186 | 343.1119 | [M + H]⁻ |
| | pyrrolo[2,3-d]pyrimidin-4-yl]-2- | | | | |
| | fluorobenzonitrile | | | | |
| 176 | 4-{4-[(3,3-difluoropyrrolidin-1-yl)methyl]-2- | C17H18F2N6 | 344.1561 | 343.1486 | [M + H]⁻ |
| | methyl-7H-pyrrolo[2,3-d]pyrimidin-5- | | | | |
| | yl}pyridin-2-amine | | | | |
| 177 | 4-{4-[(3,3-difluoropiperidin-1-yl)methyl]-2- | C18H20F2N6 | 358.1718 | 357.1622 | [M + H]⁻ |
| | methyl-7H-pyrrolo[2,3-d]pyrimidin-5- | | | | |
| | yl}pyridin-2-amine | | | | |
| 178 | 4-[4-(2,3-dihydro-1H-indol-1-ylmethyl)-2- | C21H20N6 | 356.1749 | 355.1683 | [M + H]⁻ |
| | methyl-7H-pyrrolo[2,3-d]pyrimidin-5- | | | | |
| | yl]pyridin-2-amine | | | | |
| 179 | 4-[4-(1,3-benzodioxol-5-yl)-2-methyl-7H- | C19H15N5O2 | 345.1226 | 344.1127 | [M + H]⁻ |
| | pyrrolo[2,3-d]pyrimidin-5-yl]pyridin-2-amine | | | | |
| 180 | 4-[4-(3,5-difluorophenyl)-2-methyl-7H- | C18H13F2N5 | 337.1139 | 336.1057 | [M + H]⁻ |
| | pyrrolo[2,3-d]pyrimidin-5-yl]pyridin-2-amine | | | | |
| 181 | 4-{2-methyl-4-[3-(trifluoromethoxy)phenyl]- | C19H14F3N5O | 385.1150 | 384.1086 | [M + H]⁻ |
| | 7H-pyrrolo[2,3-d]pyrimidin-5-yl}pyridin-2- | | | | |
| | amine | | | | |
| 182 | 4-[4-(1-benzothiophen-2-yl)-2-methyl-7H- | C20H15N5S | 357.1048 | 356.0969 | [M + H]⁻ |
| | pyrrolo[2,3-d]pyrimidin-5-yl]pyridin-2-amine | | | | |
| 183 | 4-[4-(1-benzofuran-2-yl)-2-methyl-7H- | C20H15N5O | 341.1277 | 340.1217 | [M + H]⁻ |
| | pyrrolo[2,3-d]pyrimidin-5-yl]pyridin-2-amine | | | | |
| 184 | 4-[2-methyl-4-(5-methyl-1-benzothiophen-2- | C21H17N5S | 371.1205 | 372.1210 | [M + H]⁺ |
| | yl)-7H-pyrrolo[2,3-d]pyrimidin-5-yl]pyridin- | | | | |
| | 2-amine | | | | |
| 185 | 4-[4-(7-chloro-1-benzothiophen-2-yl)-2- | C20H14ClN5S | 391.0658 | 392.0712 | [M + H]⁺ |
| | methyl-7H-pyrrolo[2,3-d]pyrimidin-5- | | | | |
| | yl]pyridin-2-amine | | | | |
| 186 | 4-[2-methyl-4-(1-methyl-1H-indol-2-yl)-7H- | C21H18N6 | 354.1593 | 353.1534 | [M + H]⁻ |
| | pyrrolo[2,3-d]pyrimidin-5-yl]pyridin-2-amine | | | | |
| 187 | 4-[4-(3,4-dihydronaphthalen-2-yl)-2-methyl- | C22H19N5 | 353.1640 | 352.1583 | [M + H]⁻ |
| | 7H-pyrrolo[2,3-d]pyrimidin-5-yl]pyridin-2- | | | | |
| | amine | | | | |
| 188 | 4-[2-methyl-4-(1,2,3,4-tetrahydronaphthalen- | C22H21N5 | 355.1797 | 354.1732 | [M + H]⁻ |
| | 2-yl)-7H-pyrrolo[2,3-d]pyrimidin-5- | | | | |
| | yl]pyridin-2-amine | | | | |
| 189 | 4-{2-methyl-4-[(2S)-1,2,3,4- | C22H21N5 | 355.1797 | 354.1716 | [M + H]⁻ |
| | tetrahydronaphthalen-2-yl]-7H-pyrrolo[2,3- | | | | |
| | d]pyrimidin-5-yl}pyridin-2-amine | | | | |
| 190 | 4-{2-methyl-4-[(2R)-1,2,3,4- | C22H21N5 | 355.1797 | 354.1728 | [M + H]⁻ |
| | tetrahydronaphthalen-2-yl]-7H-pyrrolo[2,3- | | | | |
| | d]pyrimidin-5-yl}pyridin-2-amine | | | | |
| 191 | 4-[4-(7-fluoro-1,3-benzodioxol-5-yl)-2- | C19H14FN5O2 | 363.1132 | 362.1022 | [M + H]⁻ |
| | methyl-7H-pyrrolo[2,3-d]pyrimidin-5- | | | | |
| | yl]pyridin-2-amine | | | | |
| 192 | 4-[4-(1,3-benzodioxol-5-yl)-2-methyl-7H- | C19H16N6O2 | 360.1335 | 361.1420 | [M + H]⁺ |
| | pyrrolo[2,3-d]pyrimidin-5-yl]pyridine-2,6- | | | | |
| | diamine | | | | |
| 193 | 4-(2-methyl-4-{[2-(trifluoromethyl)phenoxy]methyl}- | C20H16F3N5O | 399.1307 | 398.1246 | [M + H]⁻ |
| | 7H-pyrrolo[2,3- | | | | |
| | d]pyrimidin-5-yl)pyridin-2-amine | | | | |
| 194 | 4-{4-[(2-fluorophenyl)ethynyl]-2-methyl-7H- | C20H14FN5 | 343.1233 | 342.1116 | [M + H]⁻ |
| | pyrrolo[2,3-d]pyrimidin-5-yl}pyridin-2- | | | | |
| | amine | | | | |
| 195 | 4-[2-methyl-4-(5,6,7,8-tetrahydronaphthalen- | C22H21N5 | 355.1797 | 356.1805 | [M + H]⁺ |
| | 2-yl)-7H-pyrrolo[2,3-d]pyrimidin-5- | | | | |
| | yl]pyridin-2-amine | | | | |
| 196 | 4-[4-(cyclopropylethynyl)-2-methyl-7H- | C17H16N6 | 304.1436 | 305.1458 | [M + H]⁺ |
| | pyrrolo[2,3-d]pyrimidin-5-yl]pyridine-2,6- | | | | |
| | diamine | | | | |
| 197 | 4-{4-[(2-methoxyphenyl)ethynyl]-2-methyl- | C21H17N5O | 355.1433 | 356.1442 | [M + H]⁺ |
| | 7H-pyrrolo[2,3-d]pyrimidin-5-yl}pyridin-2- | | | | |
| | amine | | | | |
| 198 | 4-[4-(cyclopropylethynyl)-2-methyl-7H- | C17H15N5 | 289.1327 | 288.1228 | [M + H]⁻ |
| | pyrrolo[2,3-d]pyrimidin-5-yl]pyridin-2-amine | | | | |
| 199 | 4-(2-methyl-4-{3-[3-(piperidin-1- | C26H30N6O | 442.2481 | 443.2474 | [M + H]⁺ |
| | yl)propoxy]phenyl}-7H-pyrrolo[2,3- | | | | |
| | d]pyrimidin-5-yl)pyridin-2-amine | | | | |
| 200 | 4-(2-methyl-4-{3-[3-(4-methylpiperazin-1- | C26H31N7O | 457.2590 | 456.2477 | [M + H]⁻ |
| | yl)propoxy]phenyl}-7H-pyrrolo[2,3- | | | | |
| | d]pyrimidin-5-yl)pyridin-2-amine | | | | |
| 201 | 4-{4-[3-(2-chloroethoxy)phenyl]-2-methyl- | C20H18ClN5O | 379.1200 | 378.1140 | [M + H]⁻ |
| | 7H-pyrrolo[2,3-d]pyrimidin-5-yl}pyridin-2- | | | | |
| | amine | | | | |
| 202 | 4-(2-methyl-4-{3-[2-(pyrrolidin-1- | C24H26N6O | 414.2168 | 415.2165 | [M + H]⁺ |
| | yl)ethoxy]phenyl}-7H-pyrrolo[2,3- | | | | |
| | d]pyrimidin-5-yl)pyridin-2-amine | | | | |
| 203 | 4-(4-{3-[2-(dimethylamino)ethoxy]phenyl}- | C22H24N6O | 388.2012 | 389.1996 | [M + H]⁺ |
| | 2-methyl-7H-pyrrolo[2,3-d]pyrimidin-5- | | | | |
| | yl)pyridin-2-amine | | | | |
| 204 | 4-(2-methyl-4-{3-[2-(morpholin-4- | C24H26N6O2 | 430.2117 | 431.2096 | [M + H]⁺ |
| | yl)ethoxy]phenyl}-7H-pyrrolo[2,3- | | | | |
| | d]pyrimidin-5-yl)pyridin-2-amine | | | | |

Example 160 was prepared from Example 159 using method described in Preparation 5. Example 188 was prepared from Example 187 using method described in Preparation 5. Example 189 and 190 were prepared from Example 188 by preparative HPLC with a chiral stationary phase. Example 191 was prepared from 2-(7-fluoro-1,3-benzodioxol-5-yl)-4,4,5,5-tetramethyl-1,3,2-dioxaborolane prepared from 6-bromo-4-fluoro-1,3-benzodioxole following the procedure described in Preparation 14.

¹H NMR (399 MHz, Chloroform-d) δ 7.18 (d, 1H), 7.08 (s, 1H), 6.05 (s, 2H), 1.35 (s, 12H).

### General Procedure XIX

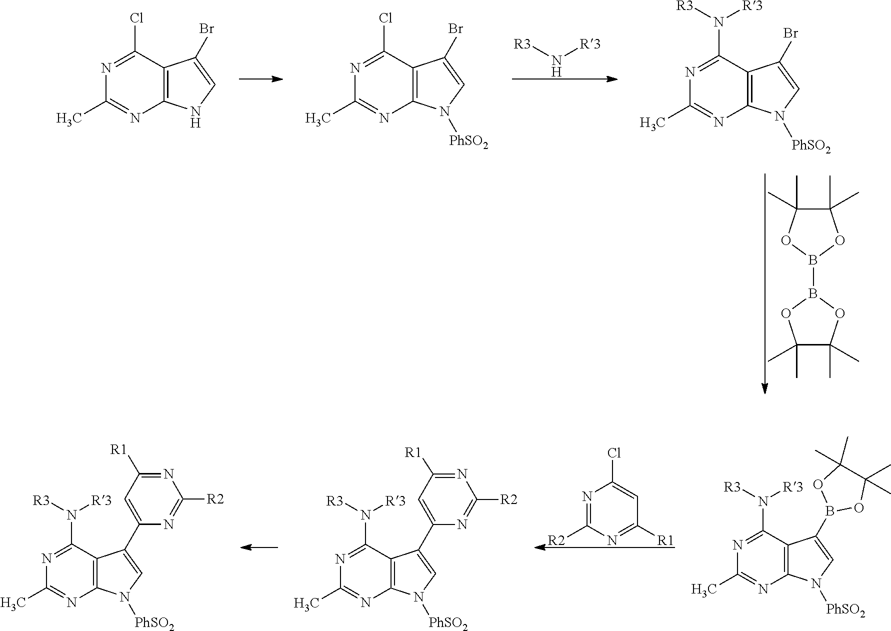

### General Procedure XX

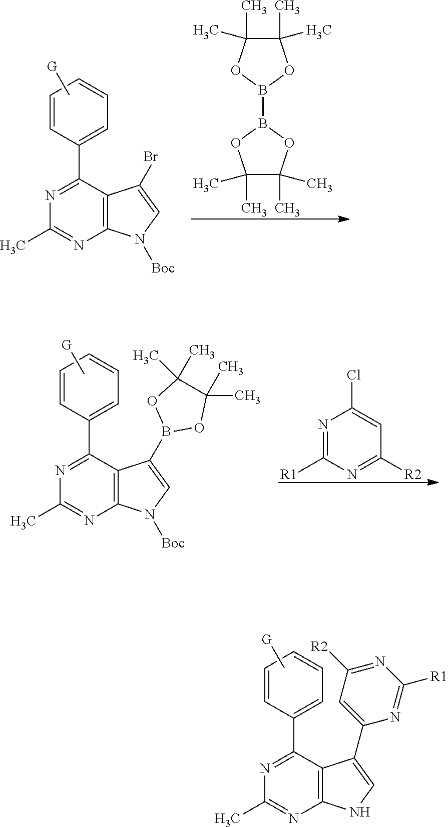

### General Procedure XXI

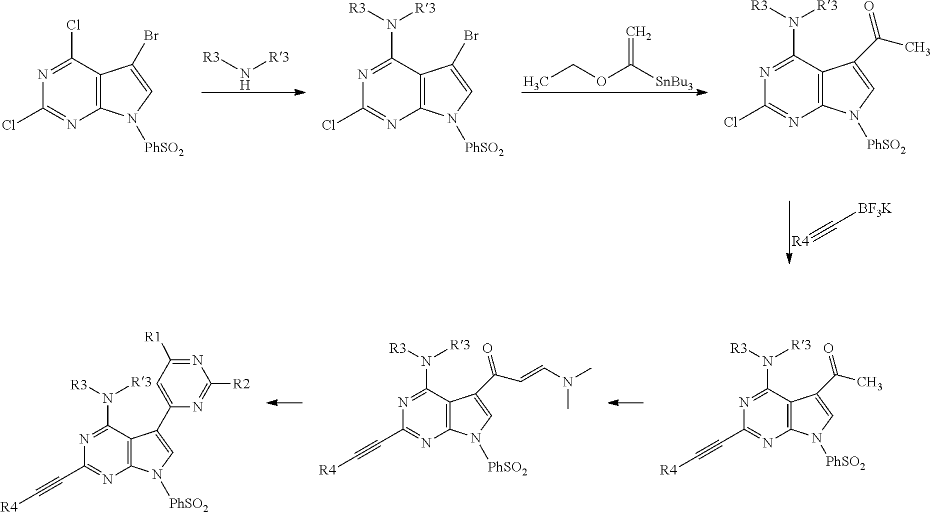

In General Procedures XIX, XX and XXI:
R₁ and R₂ are as defined in formula (I),
R₃ represents a hydrogen atom, a linear or branched (C₁-C₆)alkyl group, —(C₀-C₆)alkylene-Cy₁, —(C₀-C₆)alkylene-Cy₁-Cy₂, —(C₀-C₆)alkylene-Cy₁-O—(C₁-C₆)alkylene-Cy₂, it being understood that Cy₁ and Cy₂, independently of one another, represent a cycloalkyl group, a heterocycloalkyl group, an aryl or an heteroaryl group,
and R′₃ represents a hydrogen atom or a linear or branched (C₁-C₆)alkyl group,
or R₃ and R′₃ with the nitrogen atom carrying them a heterocycloalkyl or an heteroaryl,
   R₄ represents a hydrogen atom, a linear or branched (C₁-C₆)alkyl group or a cycloalkyl group,
   G represents a group selected from the list of substituents defined in formula (I), it being understood that the phenyl may be substituted by from 1 to 4 independent G groups.

### EXAMPLE 206

### 5-(2-aminopyrimidin-4-yl)-N-(2,6-difluorobenzyl)-2-methyl-7H-pyrrolo-[2,3-d]pyrimidin-4-amine

### Step 1 : 7-(benzenesulfonyl)-5-bromo-4-chloro-2-methyl-7H-pyrrolo-[2,3-d]pyrimidine

Starting from 4-chloro-2-methyl-7H-pyrrolo-[2,3-d]pyrimidine (1 g, 4.06 mmol) following procedure described in Preparation 19, the desired product (1.264 g, 3.27 mmol, 81%) was obtained as a white solid.

¹H NMR (399 MHz, DMSO-d) δ 8.31 (s, 1H), 8.24-8.16 (m, 2H), 7.85-7.78 (m, 1H), 7.73-7.65 (m, 2H), 2.69 (s, 3H).

LC/MS (method B): RT=1.46 min; m/z=387 [M+H]⁺

### Step 2: 7-(benzenesulfonyl)-5-bromo-N-[(2,6-difluorophenyl)methyl]-2-methyl-7H-pyrrolo [2,3]-pyrimidin-4-amine

Starting from the compound obtained in Step 1 (1.2 g, 3.10 mmol) and (2,6-difluorophenyl)methanamine (2 eq) following procedure described in Preparation 8, the desired product (1.410 g, 2.86 mmol, 92%) was obtained as a white solid.

LC/MS (method B): RT=1.52 min; m/z=493 [M+H]⁺

### Step 3: 7-(benzenesulfonyl)-N-[(2,6-difluorophenyl)methyl]-2-methyl-5-(tetramethyl-1,3,2-dioxaborolan-2-yl)-7H-pyrrolo-[2,3-d]pyrimidin-4-amine (Preparation 28)

To a solution of the compound obtained in Step 2 (1 g, 2.03 mmol) in THF (5 mL) was added bis(pinacolato)diboron (1.2 eq), KOAc (3 eq) and PdCl₂(PPh₃)₂ (10% wt). The resulting mixture was degassed under N₂ for 5 minutes before heated at 140° C. on a CEM microwave reactor for 1 hour. The reaction mixture was filtered through a plug of celite, washed with EtOAc. The organic layer was washed with brine, dried over MgSO₄ and conc. in vacuo. The residue was purified via flash chromatography using EtOAc and isohexane as eluent to give the desired product (0.675 g, 1.25 mmol, 62%) as a white solid.

LC/MS (method B): RT=1.63 min; m/z=541 [M+H]⁺

Step 4: 5-(2-aminopyrimidin-4-yl)-N-(2,6-difluorobenzyl)-2-methyl-7-(benzenesulfonyl)-7H-pyrrolo-[2,3-d]pyrimidin-4-amine

Starting from the compound obtained in Step 3 (0.915 g, 1.69 mmol) and 4-chloropyrimidin-2-amine (1.5 eq) following procedure described in Preparation 3, the product (0.551 g, 1.08 mmol, 64%) was obtained as a pale brown solid.

¹H NMR (399 MHz, DMSO-d) δ 10.79 (t, 1H), 8.44 (s, 1H), 8.29 (d, 1H), 8.20-8.13 (m, 2H), 7.80 (m, 1H), 7.65 (t, 1H), 7.40-7.24 (m, 2H), 7.01 (t, 2H), 6.70 (s, 2H), 4.90 (d, 2H), 2.38 (s, 3H).

LC/MS (method B): RT=1.41 min; m/z=508 [M+H]⁺

### Step 5: 5-(2-aminopyrimidin-4-yl)-N-(2,6-difluorobenzyl)-2-methyl-7H-pyrrolo-[2,3-d]pyrimidin-4-amine

Starting from the compound obtained in Step 4 (0.551 g, 1.08 mmol) following procedure described in Preparation 20, the desired product (0.159 g, 0.432 mmol, 40%) was obtained as a pale orange solid.

¹H NMR (399 MHz, DMSO-d) δ 11.97 (s, 1H), 10.63 (s, 1H), 8.14 (d, 1H), 8.04 (s, 1H), 7.33 (m, 1H), 7.12 (d, 1H), 7.06 (q, 2H), 6.35 (s, 2H), 4.91 (d, 2H), 2.36 (s, 3H),

LC/MS (method B): RT=0.96 min; m/z=368 [M+H]⁺

### EXAMPLE 208

### 5-(2-aminopyrimidin-4-yl)-N-(1,3-benzodioxol-4-ylmethyl)-2-methyl-7H-pyrrolo-[2,3-d]pyrimidin-4-amine

### Step 1: 7-(benzenesulfonyl)-5-bromo-4-chloro-2-methyl-7H-pyrrolo-[2,3-d]pyrimidine

Starting from 4-chloro-2-methyl-7H-pyrrolo-[2,3-d]pyrimidine (1 g, 4.06 mmol) following procedure described in Preparation 19, the desired product (1.264 g, 3.27 mmol, 81%) was obtained as a white solid.

¹H NMR (399 MHz, DMSO-d) δ 8.31 (s, 1H), 8.24-8.16 (m, 2H), 7.85-7.78 (m, 1H), 7.73-7.65 (m, 2H), 2.69 (s, 3H).

LC/MS (method B): RT=1.46 min; m/z=387 [M+H]⁺

Step 2: 7-(benzenesulfonyl)-N-(1,3-benzodioxol-4-ylmethyl)-5-bromo-2-methyl-7H-pyrrolo-[2,3-d]pyrimidin-4-amine

Starting from the compound obtained in Step 1 (0.5 g, 1.29 mmol) and 1,3-benzodioxol-4-ylmethanamine (2 eq) following procedure described in Preparation 8, the desired product (0.562 g, 112 mmol, 87%) was obtained as a white solid

¹H NMR (399 MHz, DMSO-d) δ 8.19-8.11 (m, 2H), 7.82-7.72 (m, 2H), 7.66 (dd, 2H), 7.10 (t, 1H), 6.86-6.71 (m, 3H), 6.03 (s, 2H), 4.69 (d, 2H), 2.41 (s, 3H).

LC/MS (method B): RT=1.52 min; m/z=501 [M+H]⁺

### Step 3: 7-(benzenesulfonyl)-N-3-benzodioxol-4-ylmethyl)-2-methyl-5-(tetramethyl-1,3,2-dioxaborolan-2-yl)-7H-pyrrolo-[2,3-d]pyrimidin-4-amine (Preparation 28)

To a solution of the compound obtained in Step 2 (0.25 g, 0.5 mmol) in THF (5 mL) was added bis(pinacolato)diboron (1.2 eq), KOAc (3 eq) and PdCl₂(PPh₃)₂ (10% wt). The resulting mixture was degassed under N₂ for 5 minutes before heated at 140° C. on a CEM microwave reactor for 1 hour. The reaction mixture was filtered through a plug of celite, washed with EtOAc. The organic layer was washed with brine, dried over MgSO₄ and concentrated in vacuo. The residue was purified via flash chromatography using EtOAc and isohexane as eluent to give the product (0.227 g, 0.414 mmol, 83%) as a white solid.

LC/MS (method B): RT=1.61 min; m/z=549 [M+H]⁺

### Step 4: 4-[7-(benzenesulfonyl)-4-[(1,3-benzodioxol-4-ylmethyl)amino]-2-methyl-7H-pyrrolo-[2,3-d]pyrimidin-5-yl]pyrimidin-2-amine

Starting from the compound obtained in Step 3 (227 mg, 0.414 mmol) and 4-chloropyrimidin-2-amine (1.5 eq) following procedure described in Preparation 3, the desired product (85 mg, 0.165 mmol, 40%) was obtained as a pale brow solid.

¹H NMR (399 MHz, DMSO-d) δ 9.54 (s, 2H), 8.26-8.17 (m, 2H), 7.82-7.72 (m, 1H), 7.72-7.64 (m, 3H), 7.54 (s, 2H), 6.80-6.63 (m, 3H), 6.51 (t, 1H), 5.93 (s, 2H), 4.60 (d, 2H), 2.44 (s, 3H).

LC/MS (method B): RT=1.44 min; m/z=549 [M+H]⁺

Step 5: 5-(2-aminopyrimidin-4-yl)-N-(1,3-benzodioxol-4-ylmethyl)-2-methyl-7H-pyrrolo-[2,3-d]pyrimidin-4-amine

Starting from the compound obtained in Step 4 (85 mg, 0.165 mmol) following procedure described in Preparation 20, the desired product (25 mg. 0.066 mmol, 40%) was obtained as a pale orange solid.

¹H NMR (399 MHz, DMSO-d) δ 12.00 (s, 1H), 10.55 (t, 1H), 8.14 (d, 1H), 8.06 (d, 1H), 7.13 (d, 1H), 6.91-6.72 (m, 3H), 6.22 (s, 2H), 6.03 (s, 2H), 4.81 (d, 2H), 2.37 (s, 3H).

LC/MS (method B): RT=0.935 min; m/z=376 [M+H]⁺

### EXAMPLE 210

### 4-[4-(2,2-(difluoro-1,3-benzodioxol-5-yl)-2-methyl-7H-pyrrolo-[2,3-d]pyrimidin-5-yl]pyrimidin-2-amine

### Step 1: tert-butyl 4-(2,2-difluoro-1,3-benzodioxol-5-yl)-2-methyl-5-(4,4,5,5-tetramethyl-1,3,2-dioxaborolan-2-yl)-7H-pyrrolo[2,3-d]pyrimidine-7-carboxylate

Starting from tert-butyl 5-bromo-4-(2,2-difluoro-1,3-benzodioxol-5-yl)-2-methyl-7H-pyrrolo-[2,3-d]pyrimidine-7-carboxylate (see Example 164, Step 2) (240 mg, 0.51 mmol) following procedure described in Preparation 28, the desired product (75 mg, 0.145 mmol, 28%) was obtained as a white solid.

LC/MS (method B): RT=1.62 min; m/z=516 [M+H]⁺

### Step 2: 4-[4-(2,2 difluoro-1,3-benzodioxol-5-yl)-2-methyl-7H-pyrrolo-[2,3-d]pyrimidin- 5-yl]pyrimidin-2-amine

Starting from the compound obtained in Step 1 (75 mg, 0.145 mmol) and 4-chloropyrimidin-2-amine (1.5 eq) following procedure described in Preparation 18, the desired product (7 mg, 0.018 mmol, 13%) was obtained as a white solid.

¹H NMR (399 MHz, DMSO-d) δ 12.52 (s, 1H), 8.01-7.92 (m, 2H), 7.40 (d, 1H), 7.32 (m, 1H), 7.22 (dd, 1H), 6.21 (d, 1H), 6.10 (s, 2H), 2.72 (s, 3H).

LC/MS (method B): RT=1.02 min; m/z=383 [M+H]⁺

### EXAMPLE 211

### 4-[4-(3,4-dihydroisoquinolin-2(1H)-yl)-2-ethynyl-7H-pyrrolo-[2,3-d]pyrimidin-5-yl]pyrimidin-1-amine

### Step 1: 2-[7-(benzenesulfonyl)-5-bromo-2-chloro-7H-pyrrolo-[2,3-d]pyrimidin-4-yl]-1,2,3,4-tetrahydroisoquinoline

Starting from 7-(benzenesulfonyl)-5-bromo-2,4-dichloro-7H-pyrrolo-[2,3-d]pyrimidine (prepared following procedure described in WO2007/042299) (0.875 g, 2.15 mmol) and 1,2,3,4-tetrahydroisoquinoline (2.5 eq) following procedure described in Preparation 8, the desired product (1.044 g) was obtained as a pale yellow solid (purity around 80% by LC-MS). The compound was used without further purification.

LC/MS (method B): RT=1.69 min; m/z=505 [M+H]⁺

### Step 2: 1-[7-(benzenesulfonyl)-2-chloro-4-(1,2,3,4-tetrahydroisoquinolin-2-yl)-7H-pyrrolo [2,3-d]pyrimidin-5-yl]ethan-1-one (Preparation 29)

The compound obtained in Step 1 (0.52 g, 1.03 mmol), LiCl (2.5 eq), tetrakis(triphenylphosphine)palladium (0.1 eq) and tributyl(1-ethoxyvinyl)tin (1.2 eq) were dissolved in 1,4-dioxane (10 mL) under N₂ at room temperature. The reaction mixture was stirred at 100° C. overnight under N₂. The reaction mixture was cooled to room temperature, 2N HCl (5 mL) solution was added and the reaction mixture stirred for 1 hour. The reaction mixture was diluted with sat. aq. NaHCO₃ (20 mL) solution and EtOAc (20 mL). The organic layer was separated, washed with brine, dried over MgSO₄ and concentrated in vacuo. The residue was purified via flash chromatography using EtOAc and isohexane as eluent to give the product (0.448 g). Purity around 70% by LC-MS. The compound was used without further purification.

LC/MS (method B): RT=1.55 min; m/z=467 [M+H]⁺

### Step 3: Potassium tert-butyldimethyl[2-(trifluoroboranyl)ethynyl]silane (Preparation 30)

To a solution of tert-butyldimethyl[2-(tetramethyl-1,3,2-dioxaborolan-2-yl)ethynyl]silane (0.973 g, 3.65 mmol) in acetone (15 mL) was added a solution of potassium biflouride (4 eq) in water (5 mL) at 0° C. and the suspension was allowed to warm to room temperature overnight. The reaction mixture was concentrated in vacuo and the residue was triturated with warm acetone to give the product (0.705 g, 2.86 mmol) as a white solid which was used without further purification.

¹H NMR (399 MHz, DMSO-d) δ 0.89 (s, 9H), 0.00 (s, 6H).

### Step 4: 1-[7-(benzenesulfonyl)-2-[2-(tert-butyldimethylsilyl)ethynyl]-4-(1,2,3,4-tetra hydroisoquinolin-2-yl)-7H-pyrrolo-[2,3-d]pyrimidin-5-yl]ethan-1-one

Starting from the compound obtained in Step 2 (0.400 g, 0.86 mmol) and potassium tert-butyldimethyl[2-(trifluoroboranyl)ethynyl]silane (1.78 eq) following procedure described in Preparation 10, the desired product (0.220 g, 0.35 mmol, 45%) was obtained as yellow oil.

LC/MS (method B): RT=1.75 min; m/z=571 [M+H]⁺

### Step 5: 1-[7-(benzenesulfonyl)-2-[2-(tert-butyldimethylsilyl)ethynyl]-4-(1,2,3,4-tetrahydro isoquinolin-2-yl)-7H-pyrrolo-[2,3-d]pyrimidin-5-yl]-3-(dimethylamino)prop-2-en -1-one (Preparation 31)

To a solution of the compound obtained in Step 4 (0.220 g, 0.35 mmol) in DMF (5 mL) was added N,N-dimethylformamide dimethyl acetal (6 eq) at room temperature under N₂. The reaction mixture was stirred at 90° C. for 3 hours. The mixture was cooled to room temperature, diluted with water (20 mL) and EtOAc (20 mL). The organic layer was separated, washed with brine, dried over MgSO₄ and concentrated in vacuo. The residue was purified via flash chromatography using EtOAc and isohexane as eluent to give the product (84 mg, 0.134 mmol, 35%) as a yellow oil.

LC/MS (method B): RT=1.69 min; m/z=626 [M+H]⁺

### Step 6: 4-[4-(3,4-dihydroisoquinolin-2(1H)-yl-2-ethynyl-7H-pyrrolo-[2,3-d]pyrimidin-5-yl]pyrimidin-2-amine (Preparation 32)

To a solution of the compound obtained in Step 5 (84 mg, 0.134 mmol) in THF (3 mL) was added TBAF (1M in THF solution, 1.1 eq) at 0° C. under N₂. The reaction mixture was allowed to warm to room temperature over 1 hour. The mixture was diluted with DCM (10 mL), washed with sat. aq. NaHCO₃ solution, dried over MgSO₄ and concentrated in vacuo. The residue was dissolved in butan-1-ol (3 mL), guanidine carbonate (1.5 eq) and sodium methoxide (4 eq) were added and the reaction mixture was stirred at 130° C. on a CEM microwave reactor for 30 minutes. The mixture was poured into water (10 mL) and DCM (10 mL). The organic layer was separated, washed with brine, dried over MgSO₄ and concentrated in vacuo. The crude product was purified by flash column chromatography on silica gel, during with 10% MeOH in DCM followed by preparative HPLC at pH=4 to afford the product (1.4 mg, 0.004 mmol, 3%) as a yellow solid.

¹H NMR (399 MHz, DMSO-d₆) δ 12.39 (s, 1H), 8.13 (d, 1H), 7.77 (s, 1H), 7.18-7.06 (m, 3H), 7.02-6.94 (m, 1H), 6.75 (d, 1H), 6.54 (s, 2H), 4.56 (s, 2H), 4.05 (s, 1H), 3.64 (t, 2H), 2.76 (t, 2H).

LC/MS (method B): RT=1.13 min; m/z=368 [M+H]⁺

Examples 205-212 in the following Table 5 were prepared by methods outlined in General Procedure XIX, XXI using appropriate commercially available boronate ester, amines and ethynyl. The compounds of Example 208, 210, 211 are also included.

TABLE 5
HRMS (TOF, ESI) data

| | | | Calcd Exact | | |
|---|---|---|---|---|---|
| Example | Structure | Mol Formula | Mass | Found m/z | Adduct |
| 205 | 5-(2-amino-6-methylpyrimidin-4-yl)-N-(2,6- | C19H17F2N7 | 381.1513 | 382.1569 | [M + H]⁺ |
| | difluorobenzyl)-2-methyl-7H-pyrrolo[2,3- | | | | |
| | d]pyrimidin-4-amine | | | | |
| 206 | 5-(2-aminopyrimidin-4-yl)-N-(2,6- | C18H15F2N7 | 367.1357 | 368.1413 | [M + H]⁺ |
| | difluorobenzyl)-2-methyl-7H-pyrrolo[2,3- | | | | |
| | d]pyrimidin-4-amine | | | | |
| 207 | 4-[4-(1-benzothiophen-2-yl)-2-methyl-7H- | C19H14N6S | 358.1001 | 359.1020 | [M + H]⁺ |
| | pyrrolo[2,3-d]pyrimidin-5-yl]pyrimidin-2- | | | | |
| | amine | | | | |
| 208 | 5-(2-aminopyrimidin-4-yl)-N-(1,3- | C19H17N7O2 | 375.1444 | 374.1375 | [M + H]⁻ |
| | benzodioxol-4-ylmethyl)-2-methyl-7H- | | | | |
| | pyrrolo[2,3-d]pyrimidin-4-amine | | | | |
| 209 | 4-[4-(1,3-benzodioxol-5-yl)-2-methyl-7H- | C18H14N6O2 | 346.1178 | 347.1190 | [M + H]⁺ |
| | pyrrolo[2,3-d]pyrimidin-5-yl]pyrimidin-2- | | | | |
| | amine | | | | |
| 210 | 4-[4-(2,2-difluoro-1,3-benzodioxol-5-yl)-2- | C18H12F2N6O2 | 382.0990 | 381.0912 | [M + H]⁻ |
| | methyl-7H-pyrrolo[2,3-d]pyrimidin-5- | | | | |
| | yl]pyrimidin-2-amine | | | | |
| 211 | 4-[4-(3,4-dihydroisoquinolin-2(1H)-yl)-2- | C21H17N7 | 367.1545 | 366.1442 | [M + H]⁻ |
| | ethynyl-7H-pyrrolo[2,3-d]pyrimidin-5- | | | | |
| | yl]pyrimidin-2-amine | | | | |
| 212 | 5-(6-aminopyrimidin-4-yl)-N-(2,6- | C18H15F2N7 | 367.1357 | 368.1376 | [M + H]⁺ |
| | difluorobenzyl)-2-methyl-7H-pyrrolo[2,3- | | | | |
| | d]pyrimidin-4-amine | | | | |

### General Procedure XXII

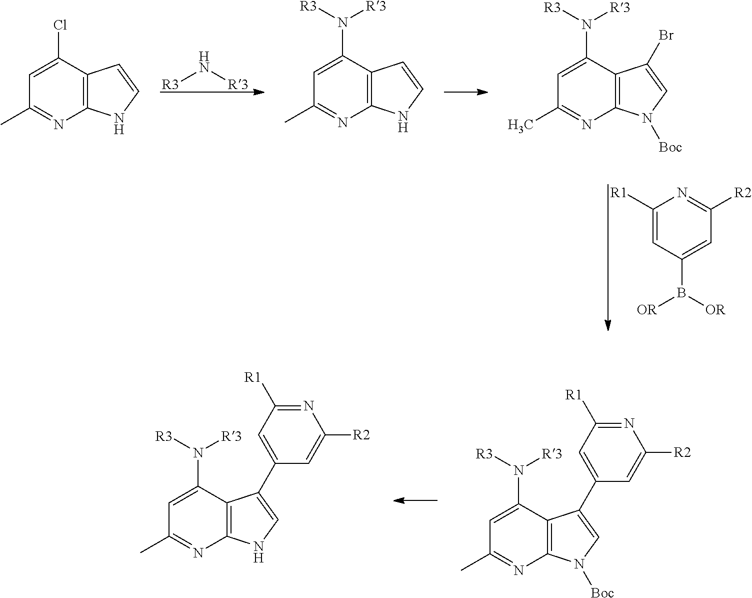

### General Procedure XXIII

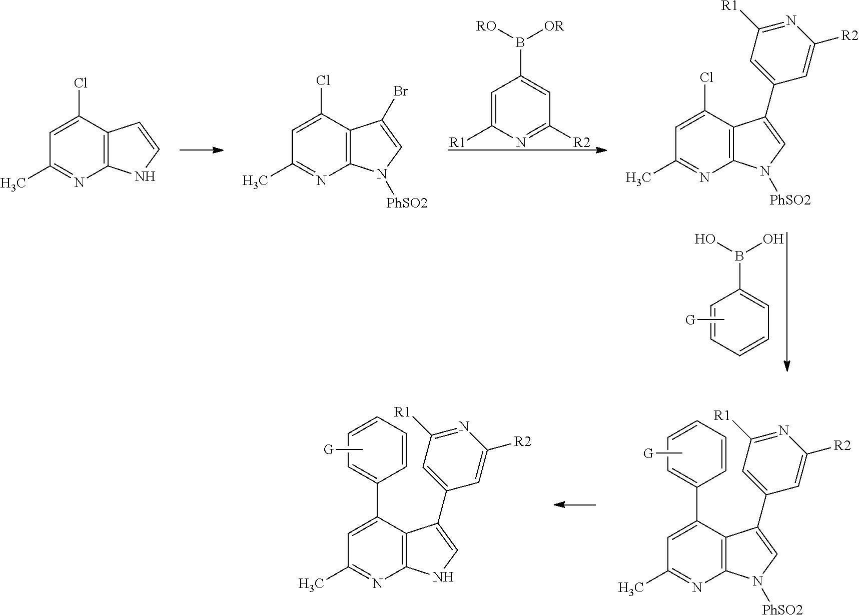

General Procedure XXIV

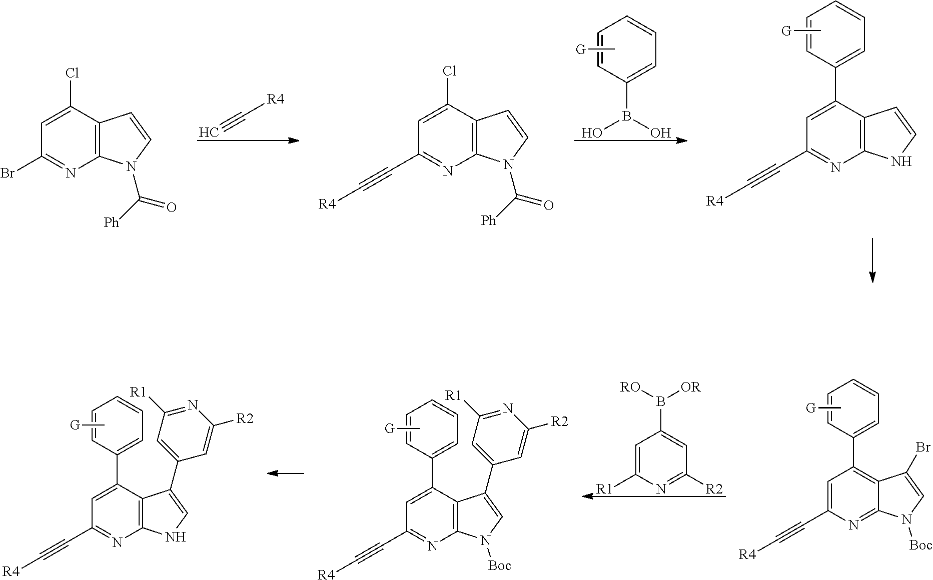

### General Procedure XXV

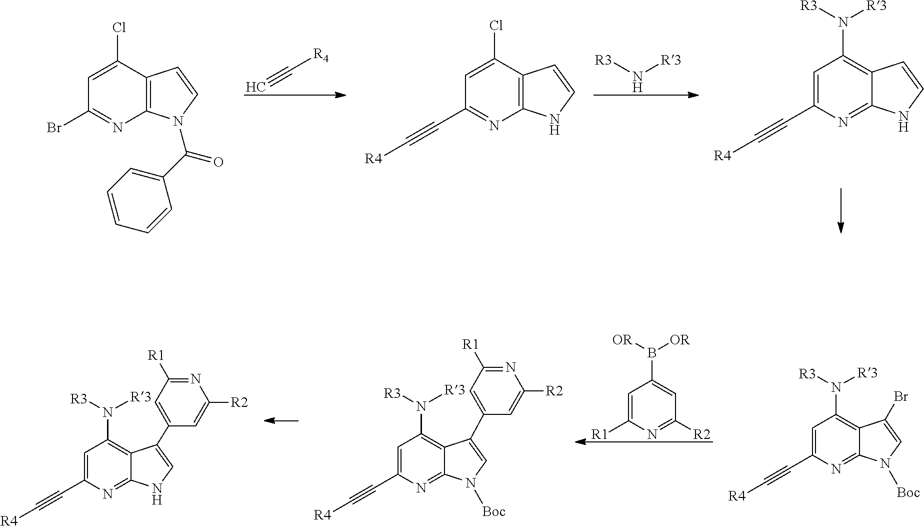

General procedure XXVI

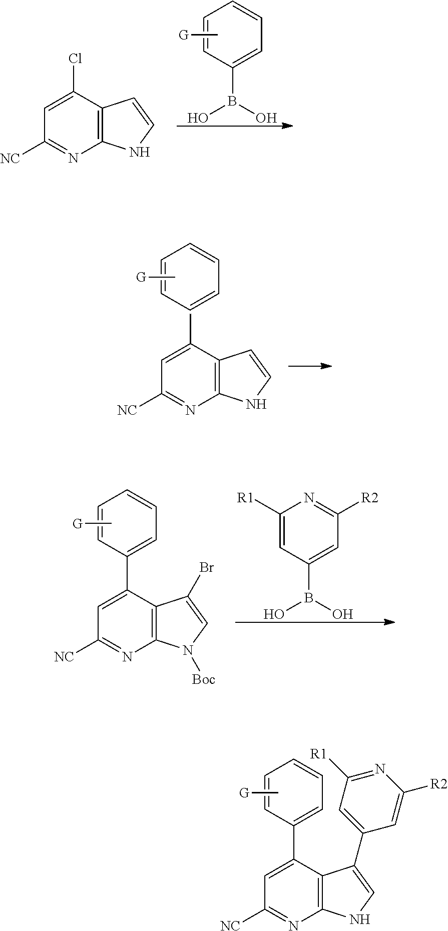

In General Procedures XXII to XXIV:
R₁ and R₂ are as defined in formula (I),
R₃ represents a hydrogen atom, a linear or branched (C₁-C₆)alkyl group, —(C₀-C₆)alkylene-Cy₁, —(C₀-C₆)alkylene-Cy₁-Cy₂, —(C₀-C₆)alkylene-Cy₁-O—(C₁-C₆)alkylene-Cy₂, it being understood that Cy₁ and Cy₂, independently of one another, represent a cycloalkyl group, a heterocycloalkyl group, an aryl or an heteroaryl group,
and R′₃ represents a hydrogen atom or a linear or branched (C₁-C₆)alkyl group,
or R₃ and R′₃ with the nitrogen atom carrying them a heterocycloalkyl or an heteroaryl,
   R₄ represents a hydrogen atom, a linear or branched (C₁-C₆)alkyl group or a cycloalkyl group,
   G represents a group selected from the list of substituents defined in formula (I), it being understood that the phenyl may be substituted by from 1 to 4 independent G groups.

### EXAMPLE 213

### 3-(2-aminopyridin-4-yl)-N-(2,6-difluorobenzyl)-6-methyl-1H-pyrrolo-[2,3-b]pyrimidin-4-amine

### Step 1: N-[(2,6-difluorophenyl)methyl]-6-methyl-1H-pyrrolo-[2,3-b]pyrimidin-4-amine (Preparation 33)

To a solution of 4-chloro-6-methyl-1H-pyrrolo-[2,3-b]pyridine (0.5 g, 3 mmol) in MeCN (15 mL) was added 2,6-difluorobenzylamine (2 eq) and pTSA.H₂O (2 eq) under N₂ at room temperature. The reaction mixture was heated at 150° C. in a CEM microwave reactor for 4 hours. The mixture was diluted with sat. aq. NaHCO₃ (20 mL) solution and EtOAc (20 mL). The organic layer was separated, washed with brine, dried over MgSO₄ and concentrated in vacuo. The residue was purified via flash chromatography using MeOH and DCM as eluent to give the product (0.521 g, 1.90 mmol, 63%) as yellow solid.

¹H NMR (399 MHz, DMSO-d6) δ 10.96 (s, 1H), 7.43 (tt, 1H), 7.20-7.08 (m, 2H), 6.95 (d, 1H), 6.77 (t, 1H), 6.53 (d, 1H), 6.15 (s, 1H), 4.44 (d, 2H), 2.35 (s, 3H).

LC/MS (method A): RT=1.82 mm; m/z=274 [M+H]⁺

### Step 2: tert-butyl 3-bromo-4-{[(2,6′-difluorophenyl)methyl]amino}-6-methyl-1H-pyrrolo [2,3-b]pyridine-1-carboxylate

Starting from the compound obtained in Step 1 (0.415 g, 1.51 mmol) following procedure described in Preparation 17, the desired product (0.280 g, 0.61 mmol, 40%) was obtained as a solid.

¹H NMR (399 MHz, Chloroform-d) δ 7.36 (s, 1H), 7.33-7.23 (m, 1H), 6.95 (t, 2H), 6.46 (s, 1H), 6.20 (d, 1H), 4.57 (d, 2H), 2.59 (s, 3H), 1.65 (s, 10 H).

LC/MS (method A): RT=2.53 min; m/z=452 [M+H]⁺

### Step 3: tert-butyl 3-(2-aminopyridin-4-yl)-4-{[(2,6-difluorophenyl)methyl]amino}-6-methyl -1H-pyrrolo-[2,3-b]pyridine-1-carboxylate

Starting from the compound obtained in Step 2 (0.280 g, 0.61 mmol) and tert-butyl N-[4-(tetramethyl-1,3,2-dioxaborolan-2-yl)pyridin-2-yl]carbamate (1.4 eq) following procedure described in Preparation 3, the desired product (0.154 g, 0.33 mmol, 53%) was obtained as an off-white solid.

LC/MS (method B): RT=0.99 min; m/z=466 [M+H]⁺

### Step 4: 3-(2-aminopyridin-4-yl)-N-(2,6-difluorobenzyl)-6-methyl-1H-pyrrolo-[2,3-b]pyridin-4-amine

Starting from the compound obtained in Step 3 (0.154 g. 0.33 mmol) following procedure described in Preparation 7, the product (0.110 g, 0.30 mmol, 91%) was obtained as a white solid.

¹H NMR (399 MHz, DMSO-d6) δ 11.43 (s, 1H), 7.85 (d, 1H), 7.42 (tt, 1H), 7.20-7.07 (m, 3H), 6.51-6.43 (m, 2H), 6.29 (s, 1H), 5.89 (s, 2H), 5.23 (t, 1H), 4.49 (4 2H), 2.39 (s, 3H).

LC/MS (method A): RT=1.58 min; m/z 366 [M+H]⁺

### EXAMPLE 214

### 4-[4-(5-fluoropyridin-3-yl)-6-methyl-1H-pyrrolo-[2,3-b]pyridin-3-yl]pyridin-2-amine

### Step 1: 1-(benzenesulfonyl)-3-bromo-4-chloro-6-methyl-1H-pyrrolo-[2,3-b]pyridine

Starting from 4-chloro-6-methyl-1H-pyrrolo-[2,3-b]pyridine (0.713 g, 4.27 mmol) following procedure described in Preparation 19, the desired product (0.493 g, 1.28 mmol, 30%) was obtained as a white solid.

¹H NMR (399 MHz, DMSO-d6) δ 8.21-8.13 (m, 3H), 7.81-7.72 (m, 1H), 7.70-7.62 (m, 2H), 7.41 (s, 1H), 2.56 (s, 3H).

LC/MS (method B): RT=1.52 min; m/z=386 [M+H]⁺

### Step 2: 4-[1-(benzenesulfonyl)-4-chloro-6-methyl-1H-pyrrolo-[2,3-b]pyridin-3-yl]pyridin-2-amine

Starting from the compound obtained in Step 1 (0.493 g, 1.28 mmol) and 4-(tetramethyl-1,3,2-dioxaborolan-2-yl)pyridin-2-amine (1.4 eq) following procedure described in Preparation 3, the desired product (0.200 g, 0.501 mmol, 39%) was obtained as a pale yellow solid.

¹H NMR (399 MHz, DMSO-d6) δ 8.27-8.17 (m, 2H), 8.00-7.91 (m, 2H), 7.81-7.63 (m, 3H), 7.38 (s, 1H), 6.64 (dd, 1H), 6.57 (d, 1H), 5.99 (s, 2H), 2.57 (s, 3H).

LC/MS (method B): RT=1.13 min; m/z=399 [M+H]⁺

### Step 3: 4-[1-(benzenesulfonyl)-4-(5-fluoropyridin-3-yl)-6-methyl-1H-pyrrolo-[2,3-b]pyridin-3-yl]pyridin-2-amine

Starting from the compound obtained in Step 2 (0.133 g. 0.33 mmol) and (5-fluoropyridin-3-yl)boronic acid (1.1 eq) following procedure described in Preparation 3, the product (97 mg, 0.211 mmol, 63%) was obtained as a pale brown solid.

¹H NMR (399 MHz, DMSO-d6) δ 8.48 (d, 1H), 8.31-8.23 (m, 2H), 8.19 (t, 1H), 7.97 (s, 1H), 7.82-7.73 (m, 1H), 7.73-7.63 (m, 2H), 7.62-7.44 (m, 4H), 7.33 (s, 1H), 6.16 (m, 1H), 5.89 (dd, 1H), 5.77 (s, 2H), 2.65 (s, 3H).

LC/MS (method B): RT=1.1 mm; m/z=460 [M+H]⁺

### Step 4: 4-[4-(5-fluoropyridin-3-yl)-6-methyl-1H-pyrrolo-[2,3-b]pyridin-3-yl]pyridin-2-amine

Starting from the compound obtained in Step 3 (97 mg, 0.211 mmol) following procedure described in Preparation 20, the desired product (20 mg, 0.06 mmol, 30%) was obtained as a white solid.

¹H NMR (399 MHz, DMSO-d6) δ 12.06 (s, 1H), 8.49 (d, 1H), 8.26 (d, 1H), 7.63 (s, 1H), 7.56-7.46 (m, 2H), 7.10 (s, 1H), 6.08 (d, 1H), 5.87 (dd, 1H), 5.62 (s, 2H), 2.61 (s, 3H).

LC/MS (method A): RT=1.67 min; m/z=320 [M+H]⁺

### EXAMPLE 215

### 4-[6-(cyclopropylethynyl)-4-(2,3-dihydro-1,4-benzodioxin-6-yl)-1H-pyrrolo-[2,3-b]pyridin-3-yl]pyridin-2-amine

### Step 1: 1-benzoyl-4-chloro-6-(cyclopropylethynyl)-1H-pyrrolo-[2,3-b]pyridine

Starting from 1-benzoyl-6-bromo-4-chloro-1H-pyrrolo-[2,3-b]pyridine (prepared following procedure described on WO2009/087225) (1.12 g, 3.72 mmol) and ethynylcyclopropane (3 eq) following procedure described in Preparation 27, the desired product (1.053 g, 3.28 mmol, 88%) was obtained as a pale brown solid.

LC/MS (method B): RT=1.52 min; m/z=321 [M+H]⁺

### Step 2: 6-6-(cyclopropylethynyl)-4-(2,3-dihydro-1,4-benzodioxin-6-yl)-1H-pyrrolo-[2,3-b]pyridine

Starting from the compound obtained in Step 1 (0.5 g, 1.56 mmol) and (2,3-dihydro-1,4-benzodioxin-6-yl)boronic acid (1.2 eq) following procedure described in Preparation 3, the desired product (0.234 g, 0.74 mmol, 47%) was obtained as a brown solid.

LC/MS (method B): RT=1.35 min; m/z=316 [M+H]⁺

### Step 3: tert-butyl 3-bromo-6-(cyclopropylethynyl)-4-(2,3-dihydro-1,4-benzodioxin-6-yl)-1H-pyrrolo-[2,3-b]pyridine-1-carboxylate

Starting from the compound obtained in Step 2 (0.234 g, 0.74 mmol) following procedure described in Preparation 17, the desired product (0.326 g, 0.658 mmol, 89%) was obtained as a pale yellow solid.

LC/MS (method B): RT=1.7 min; m/z=497 [M+H]⁺

### Step 4: tert-butyl 3-(2-{[(tert-butoxy)carbonyl]amino}pyridin-4-yl)-6-(cyclopropyl ethynyl)-4-(2,3-dihydro -1,4-benzodioxin -6-yl)-1H-pyrrolo[2,3-b]pyridine -1-carboxylate

Starting from the compound obtained in Step 3 (0.326 g, 0.658 mmol) and tert-butyl N-[4-(tetramethyl-1,3,2-dioxaborolan-2-yl)pyridin-2-yl]carbamate (1.1 eq) following procedure described in Preparation 3, the desired product (0.211 g, 0.347 mmol, 53%) was obtained as a pale yellow solid.

LC/MS (method A): RT=3.05 min; m/z=609 [M+H]⁺

### Step 5: 4-[6-(cyclopropylethynyl)-4-(2,3-dihydro-1,4-benzodioxin-6-yl)-1H-pyrrolo [2,3-b]pyridin-3-yl]pyridin-2-amine

Starting from the compound obtained in Step 4 (0.211 g, 0.347 mmol) following procedure described in Preparation 7, the desired product (54 mg, 0,132 mmol, 38%) was obtained as a white solid.

¹H NMR (399 MHz, DMSO-d6) δ 12.08 (s, 1H), 7.72 (s, 1H), 7.50 (d, 1H), 7.07 (s, 1H), 6.73-6.60 (m, 3H), 6.05 (m, 1H), 5.89 (dd, 1H), 5.52 (s, 2H), 4.20 (ddd, 4H), 1.60 (tt, 1H), 0.98-0.87 (m, 2H), 0.87-0.76 (m, 2H).

LC/MS (method A): RT=2.16; m/z=409 [M+H]⁺

### EXAMPLE 216

### 3-(2-aminopyridin-4-yl)-6-(cyclopropylethynyl-N-(2,6-difluorobenzyl)-1H-pyrrolo-[2,3-b]pyridin-4-amine

### Step 1: 4-chloro -6-(cyclopropylethynyl)-1H-pyrrolo-[2,3-b]pyridine (Preparation 34)

To a solution of 1-benzyol-6-bromo-4-chloro-1H-pyrrolo-[2,3-b]pyridine (prepared following procedure described in WO2009/087225) (1.52 g, 4.54 mmol) in Et₃N (15 ml) and THF (3 mL) was added ethynylcyclopropane (3 eq) and CuI (0.3 eq) at room temperature. The solution was purged with N₂ for 5 minutes before adding Pd(PPh₃)₂Cl₂ (0.3 eq) and the reaction mixture was stirred at room temperature overnight. Water (1 mL) was added to the reaction mixture and heated at 80° C. on CEM microwave reactor for 1 hour. The mixture was diluted with water (20 mL) and DCM (20 mL). The organic layer was separated, washed with brine, dried over MgSO₄ and concentrated in vacuo. The residue was purified via flash chromatography using MeOH and DCM as eluent followed by trituration with isohexane to give the product (0.652 g, 3 mmol, 66%) as an off-white solid.

¹H NMR (399 MHz, DMSO-d6) δ 12.04 (s, 1H), 7.65 (d, 1H), 7.24 (s, 1H), 6.50 (d, 1H), 1.59 (tt, 1H), 1.01-0.85 (m, 2H) 0.89-0.72 (m, 2H).

LC/MS (method B): RT=1.31 min; m/z=217 [M+H]⁺

### Step 2: 6-(cyclopropylethynyl)-N-[(2,6-difluorophenyl)methyl]-1H-pyrrolo-[2,3-b]pyridin-4-amine ( Preparation 35)

The compound obtained in Step 1 (0.3 g, 1.38 mmol), 2,6-difluorobenzylamine (1.2 eq), BrettPhos (0.01 eq) and BrettPhos precatalyst (0.01 eq) were added into a microwave vial. The vial was sealed with a teflon screw-cap, then evacuated and backfilled with N₂. LiHMDS (1M solution in THF, 2 eq) was added at room temperature under N₂. The reaction mixture was heated at 65° C. in a CEM microwave reactor for 4 hours. The reaction mixture was quenched with 1N HCl (2 mL) solution and diluted with DCM (50 mL). The organic layer was separated, washed with brine, dried over MgSO₄ and concentrated in vacuo. The residue was purified via flash chromatography using MeOH and DCM as eluent to give the product (0.429 g, 1.32 mmol, 96%) as a pale brown solid.

¹H NMR (399 MHz, DMSO-d6) δ 11.13 (t, 1H), 7.43 (tt, 1H), 7.20-7.06 (m, 3H), 6.94 (t, 1H), 6.59 (dd, 1H), 6.33 (s, 1H), 4.44 (d, 2H), 1.53 (tt, 1H), 0.96-0.81 (m, 2H), 0.80-0.66 (m, 2H).

LC/MS (method B): RT=1.12 min; m/z=324 [M+H]⁺

### Step 3: tert-butyl 3-bromo-6-(cyclopropylethynyl)-4-{[(2,6difluorophenyl)methyl]amino}-1H-pyrrolo-[2,3-b]pyridine-1-carboxylate

Starting from the compound obtained in Step 2 (0.429 g, 1.32 mmol) following procedure described in Preparation 17, the desired product (0.463 g, 0.921 mmol, 69%) was obtained as an off-white solid.

¹H NMR (399 MHz, Chloroform-d) δ 7.42 (s, 1H), 7.29 (m, 1H), 7.01-6.92 (m, 2H), 6.69 (s, 1H), 6.19 (t, 1H), 4.56 (d, 2H), 1.64 (s, 9H), 1.50 (m, 1H), 1.00-0.86 (m, 4H).

LC/MS (method B): RT=1.61 min; m/z=502 [M+H]⁺

### Step 4: tert-butyl 3-(2-{[(tert-butoxy)carbonyl]amino}pyridin-4-yl)-6-(cyclopropyl ethynyl)-4-{[(2,6-difluorophenyl)methyl]amino}-1H-pyrrolo[2,3-b]pyridine-1-carboxylate

Starting from the compound obtained in Step 3 (0.463 g, 0.921 mmol) and tert-butyl N-[4-(tetramethyl-1,3,2-dioxaborolan-2-yl)pyridin-2-yl]carbamate (1.1 eq) following procedure described in Preparation 3, the desired product (0.233 g, 0.378 mmol, 41%) was obtained as a pale yellow solid.

¹H NMR (399 MHz, Chloroform-d) δ 8.25-8.19 (m, 1H), 8.06 (d, 1H), 7.48 (s, 1H), 7.42 (s, 1H), 7.27-7.21 (m, 1H), 7.02 (dd, 1H), 6.95-6.85 (m, 2H), 6.72 (s, 1H), 4.86 (t, 1H), 4.45 (d, 2H), 1.67 (s, 9H), 1.55 (s, 9H), 1.53-1.48 (m, 1H), 0.97-0.82 (m, 4H).

LC/MS (method B): RT=1.64 min; m/z=616 [M+H]⁺

### Step 5: 3-(2-aminopyridin-4-yl)-6-(cyclopropylethynyl)-N-(2,6-difluorobenzyl)-1H-pyrrolo-[2,3-b]pyridin-4-amine

Starting from the compound obtained in Step 4 (0.233 g, 0.378 mmol) following procedure described in Preparation 7, the desired product (88 mg, 0.211 mmol, 56%) was obtained as a white solid.

¹H NMR (399 MHz, DMSO-d6) δ 11.61 (s, 1H), 7.85 (d, 1H), 7.18-7.36 (m, 1H), 7.32 (s, 1H), 7.14 (t, 2H) 6.50-6.42 (m, 3H), 5.91 (s, 2H), 5.31 (t, 1H), 4.48 (d, 2H), 1.56 (tt, 1H), 0.91 (m, 2H), 0.80-0.71 (m, 2H).

LC/MS (method B): RT=1.09 mm, m/z=416 [M+H]⁺

### EXAMPLE 223

### 3-(2-aminopyridin-4-yl)-4-(1,3-benzodioxol-5-yl)-1H-pyrrolo-[2,3-b]pyridin-6-carbonitrile

### Step 2: 4-(1,3-benzodioxol-5-yl)-1H-pyrrolo[2,3-b]pyridine-6-carbonitrile

Starting from 4-chloro-1H-pyrrolo-[2,3-b]pyridine-6-carbonitrile (prepared from Synthesis, 2008, (2), 201-204) (100 mg, 0.56 mmol) and (1,3-benzodioxol-5-yl)boronic acid (1.1 eq) following procedure described in Preparation 3, the desired product (84 mg, 0.32 mmol, 57%) was obtained as a yellow solid.

¹H NMR (399 MHz, DMSO-d6) δ 12.38 (s, 1H), 7.93-7.82 (m, 1H), 7.75 (s, 1H), 7.43-7.31 (m, 2H), 7.12 (d, 1H), 6.78 (dd, 1H), 6.14 (s, 2H).

LC/MS (method B): RT=1.23 min; m/z=264 [M+H]⁺

### Step 2: tert-butyl 4-(1,3-benzodioxol-5-yl)-3-bromo-6-cyano-1H-pyrrolo-[2,3-b]pyridin-1-carboxylate

Starting from the compound obtained in Step 1 (0.289 g, 1.1 mmol) following procedure described in Preparation 17, the desired product (0.373 g, 0.84 mmol, 77%) was obtained as a yellow solid.

¹H NMR (399 MHz, DMSO-d6) δ 8.33 (s, 1H), 7.87 (s, 1H), 7.14-7.04 (m, 2H), 6.98 (dd, 1H), 6.14 (s, 2H), 1.64 (s, 9H).

### Step 3: 3-(2-aminopyridin-4-yl)-4-(1,3-benzodioxol-5-yl)-1H-pyrrolo[2,3-b]pyridine -6-carbonitrile

Starting from the compound obtained in Step 2 (0.180 g, 0.41 mmol) and tert-butyl N-[4-(tetramethyl-1,3,2-dioxaborolan-2-yl)pyridin-2-yl]carbamate (1.1 eq) following procedure described in Preparation 3. The crude reaction mixture was concentrated in vacuo and the residue dissolved in DCM (2mL) and TFA (1.5 mL) following procedure described in Preparation 7. The crude reaction mixture was concentrated in vacuo and the residue was triturated with MeOH to give the product (49 mg, 0.137 mmol, 34%) as a TFA salt.

¹H NMR (399 MHz, DMSO-d6) δ 13.10 (d, 2H), 8.38 (d, 1H), 7.79 (s, 1H), 7.67 (t, 3H), 6.98 (d, 1H), 6.85 (d, 1H), 6.71 (dd, 1H), 6.49-6.30 (m, 2H), 6.05 (s, 2H).

LC/MS (method B): RT=0.97 min; m/z=356 [M+H]⁺

Examples 213-225 in the following Table 6 were prepared by methods outlined in General Procedure XXII-XXVI using appropriate commercially available boronate ester, amines and ethynyl. The compounds of Example 213, 214, 215, 216, 223 are also included.

TABLE 6
HRMS (TOF, ESI) data

| | | | Calcd Exact | | |
|---|---|---|---|---|---|
| Example | Structure | Mol Formula | Mass | Found m/z | Adduct |
| 213 | 3-(2-aminopyridin-4-yl)-N-(2,6-difluoro | C20H17F2N5 | 365.1452 | 366.1514 | [M + H]⁺ |
| | benzyl)-6-methyl-1H-pyrrolo[2,3-b]pyridin- | | | | |
| | 4-amine | | | | |
| 214 | 4-[4-(5-fluoropyridin-3-yl)-6-methyl-1H- | C18H14FN5 | 319.1233 | 320.1299 | [M + H]⁺ |
| | pyrrolo[2,3-b]pyridin-3-yl]pyridin-2-amine | | | | |
| 215 | 4-[6-(cyclopropylethynyl)-4-(2,3-dihydro- | C25H20N4O2 | 408.1586 | 409.1618 | [M + H]⁺ |
| | 1,4-benzodioxin-6-yl)-1H-pyrrolo[2,3- | | | | |
| | b]pyridin-3-yl]pyridin-2-amine | | | | |
| 216 | 3-(2-aminopyridin-4-yl)-6-(cyclopropylethynyl)- | C24H19F2N5 | 415.1609 | 416.1638 | [M + H]⁺ |
| | N-(2,6-difluorobenzyl)-1H- | | | | |
| | pyrrolo[2,3-b]pyridin-4-amine | | | | |
| 217 | 4-[4-(2,3-dihydro-1,4-benzodioxin-6-yl)-6- | C21H18N4O2 | 358.1430 | 359.1428 | [M + H]⁺ |
| | methyl-1H-pyrrolo[2,3-b]pyridin-3- | | | | |
| | yl]pyridin-2-amine | | | | |
| 218 | 4-[4-(1,3-benzodioxol-5-yl)-6- | C24H19N5O2 | 409.1539 | 410.1570 | [M + H]⁺ |
| | (cyclopropylethynyl)-1H-pyrrolo[2,3- | | | | |
| | b]pyridin-3-yl]pyridine-2,6-diamine | | | | |
| 219 | 4-[4-(1,3-benzodioxol-5-yl)-6- | C24H18N4O2 | 394.1430 | 395.1430 | [[M + H]⁺ |
| | (cyclopropylethynyl)-1H-pyrrolo[2,3- | | | | |
| | b]pyridin-3-yl]pyridin-2-amine | | | | |
| 220 | 4-[4-(1,3-benzodioxol-5-yl)-6-ethynyl-1H- | C21H14N4O2 | 354.1117 | 355.1120 | [M + H]⁺ |
| | pyrrolo[2,3-b]pyridin-3-yl]pyridin-2-amine | | | | |
| 221 | 4-[4-(1,3-benzodioxol-5-yl)-6-ethynyl-1H- | C21H15N5O2 | 369.1226 | 368.1146 | [M + H]⁻ |
| | pyrrolo[2,3-b]pyridin-3-yl]pyridine-2,6- | | | | |
| | diamine | | | | |
| 222 | 4-[4-(1,3-benzodioxol-5-yl)-6-methyl-1H- | C20H16N4O2 | 344.1273 | 343.1191 | [M + H]⁻ |
| | pyrrolo[2,3-b]pyridin-3-yl]pyridin-2-amine | | | | |
| 223 | 3-(2-aminopyridin-4-yl)-4-(1,3-benzodioxol- | C20H13N5O2 | 355.1069 | 354.1014 | [M + H]⁻ |
| | 5-yl)-1H-pyrrolo[2,3-b]pyridine-6- | | | | |
| | carbonitrile | | | | |
| 224 | 4-(1,3-benzodioxol-5-yl)-3-(2,6-diamino | C20H14N6O2 | 370.1178 | 371.1170 | [M + H]⁺ |
| | pyridin-4-yl)-1H-pyrrolo[2,3-b]pyridine-6- | | | | |
| | carbonitrile | | | | |
| 225 | 4-[6-methyl-4-(4-methyl-3,4-dihydro-2H- | C22H21N5O | 371.1746 | 372.1738 | [M + H]⁺ |
| | 1,4-benzoxazin-6-yl)-1H-pyrrolo[2,3- | | | | |
| | b]pyridin-3-yl]pyridin-2-amine | | | | |

### PHARMACOLOGICAL STUDY

### EXAMPLE A

### Kinase TR-FRET Assays

Inhibition of the enzymatic activity of human kinases was evaluated in a Time-Resolved Fluorescence Resonance Energy Transfer (TR-FRET) assay in 384-well reaction plates. In this assay, full-length human kinases from Carna Biosciences—DYRK1A (NM_001396, ref. 04-130; 2.0 ng/μl), DYRK1B (NM_004714, ref. 04-131; 1.2 ng/μl), CLK1 (NM_001162407, ref. 04-126; 0.7 ng/μl), CDK9 (NM001261, ref. 04-110; 0.9 ng/μl), or GSK3β (NM_001146156, ref. 04-141; 2.0 ng/μl)—were incubated for 40 minutes (DYRK1A and DYRK1B) or 100 minutes (CLK1, CDK9 and GSK3β) at room temperature with ATP (Sigma A_{2383, 10} μM) and a ULight™-labelled human Myelin Basic Protein (MBP) peptide substrate (Perkin Elmer TRF0109, 100 nM) in a reaction buffer composed of 50 mM HEPES pH7.4, 1 mM EGTA, 10 mM MgCl₂, 2 mM DTT and 0.01% Tween20. Test compounds of the invention were added in reaction buffer at a range of concentrations from 0.1 nM to 30 μM. Following addition of EDTA (Sigma E7889, 10 mM) to stop the reaction. Europium-labelled mouse monoclonal antibody recognizing phospho-Thr232 in MBP (Perkin Elmer TRF0201, 1 nM) was added. After one hour, the reaction plates were read using a fluorescence reader (EnVision®, Perkin Elmer) at 620 nm and 665 nm (excitation at 340 mn): when the Europium donor fluorophore is excited by light at 340 mn, an energy transfer (620 mn) to the acceptor occurs, which will then emit light at 665 nm. The activity, and hence inhibition, of DYRK1A kinase activity is thus measured by the relative intensity of the emitted light. The IC₅₀ was calculated from the concentration-activity curve as the concentration of the test compound required for 50% inhibition of kinase activity. The results are presented in Table 1.

### EXAMPLE B

### Kinase ADP Assays

The activity of His-TEV-DYRK1A Kinase domain (aa127-485) was measured using the accumulation of ADP produced during the the phosphorylation of the peptide substrate Woodtide (Zinnsser Analytic) using ATP (Sigma Aldrich A7699). The enzyme reaction was conducted in assay buffer (pH 7.4), containing 15 mM Hepes; 20 mM NaCl; 1 mM EGTA; 10 mM MgCl₂; 0.02% Tween20 and 0.1 mg/ml Bovine-y-globulin. Test compounds of the invention were added in reaction buffer in a range of concentrations for 10 minutes at 30° C. in the presence of 20 nM DYRK1A enzyme, 40 μM peptide substrate and 20 μM ATP. Detection reagents (DiscoveRx 90-0083), ADP Hunter Plus Reagent A and then ADP Hunter Plus Reagent B were added. After a following 20 minutes incubation at 30° C., ADP Hunter Plus Stop Solution was added. The fluorescence intensity was measured at 590 nm. The IC₅₀ was calculated from the concentration-activity curve as the concentration of the test compound required for 50% inhibition of kinase activity. The results are presented in Table 1.

### EXAMPLE C

### Cellular DYRK1A Autophosphorylation Assay

On day 0, human U2-OS osteosarcoma cells were seeded in 12-well culture plates (100,000 cells per well) and incubated at 37° C. in the presence of 5% CO₂ in 1 ml McCoy's 5A (Modified) medium containing GlutaMAX™ (Gibco 36600), supplemented with 50 units/ml penicillin, 50 μg/ml streptomycin, 10 mM Hepes buffer, pH=7.4, and 10% foetal calf serum (FCS, Sigma F7524). On day 1, medium was replaced with 500 μl Optimem medium containing GlutaMAX™ (Gibco 51985), 150 ng of a pcDNA3.1 plasmid (Invitrogen) containing a sequence coding for full-length, wild-type human DYRK1A (NM_001396) with an HA tag, 0.3% lipofectamine (Invitrogen 18324-020), and 0.6% Plus reagent (Invitrogen Cat No 11514-015). After 5 hours, medium was replaced with 900 μl McCoy's 5A (Modified) medium containing GlutaMAX™ (Gibco 36600). On day 2, cells were exposed to a range of concentrations of the test compounds of the invention for 5 hours. Cells were then washed in phosphate-buffered saline solution and cell lysed in lysis buffer comprised of 150 mM NaCl, 20 mM Tris-HCl pH 7.4, 1% triton X-100, 1 mM EGTA, 1 mM EDTA and protease (1% v/v; 539134; Calbiochem) and phosphatase (1% v/v; 524625; Calbiochem) inhibitor cocktails (50 μl lysis buffer/well). The relative levels of phospho-Ser520-DYRK1A were assayed using either western blotting or the Mesoscale ELISA platform. For analysis by western blot, lysates were diluted into Laemmli sample buffer (Bio-Rad) containing 5% v/v β-mecaptoethanol, heated for 5 min at 95° C., and resolved on Tris-glycine gels or NuPage Bis-Tris gels (Novex; Invitrogen). Biotinylated molecular weight, standards (Cell Signaling Technology) were included in all gels. Proteins were transferred to nitrocellulose membranes (Hybond, ECL; Amersham), which were blocked in Tris-buffered saline/0.1% tween 20 (TBST) containing 5% milk, and probed at 4° C. overnight with anti-phospho-Ser520-DYRK1A antibody (Eurogentec SE6974-75; 0.23 μg/ml 5% BSA) or anti DYRK1A antibody (Abnova H00001859; 0.5 μg/ml in 5% milk). Peroxidase-conjugated secondary antibodies were diluted into 5% milk and applied to membranes for 1 h at 20° C. Chemiluminescence detection was performed using the ECL plus western blotting dejection kit (Amersham) and was recorded on ECL plus hyperfilm (Amersham). Blots were scanned using the Bio-Rad GS-800 calibrated densitometer and quantitative analysis of western blots was performed using TotalLab software (Amersham). IC₅₀ values for inhibition of phospho-Ser520-DYRK1A were calculated from dose-response curves plotting the ratio between phospho-Ser520-DYRK1A and total DYRK1A signals at each concentration. For analysis by Mesoscale ELISA, lysates were transferred to BSA-blocked ELISA plates with pre-bound anti-HA capture antibodies (Novus biological NB600-364; 15 μg/ml) for 1 hour with shaking at RT. Anti-phospho-Ser520-DYRK1A antibody (Eurogentec SE6974-75; 2.3-3.0 mg/ml) and anti DYRK1A antibody (Abnova H00001859; 3 μg/ml) was then added for 1 hour at RT, followed by addition of Sulfa-TAG anti-rabbit detection antibody (ref MSD R₃₂AB; 1 μg/ml) and Sulfa-TAG anti-mouse detection antibody (ref MSD R₃₂-AC-1; 1 μg/ml). After a further 1hour, Read Buffer was added and plates were read on the Sector Imager 2400 (Mesoscale). IC₅₀ values for inhibition of phospho-Ser520-DYRK1A were calculated from dose-response curves. The results showed that the compounds of the invention are powerful inhibitors of cellular DYRK1A Ser520 autophosphorylation. The results are presented in Table 1.

### EXAMPLE D

### Pharmacodynamic Assay in Tumor Xenografts for Inhibition of DYRK1A Autophosphorylation

For pharmacodynamics studies of inhibition of DYRK1A autophosphorylation, female SCID mice were injected subcutaneously with RS4;11 human acute lymphoblastic leukemia cells. When tumors reached a size of 200-300 mm³, mice were randomized into homogeneous groups of 3 and given a single oral administration of the compounds of the invention at doses of up to 100 mg/kg. At various times after treatment, typically 2 hours and 6 hours, treated and control mice were sacrificed, tumors were excised and proteins were extracted in tissue lysis buffer comprised of 150 mM NaCl, 20 mM Tris-HCl pH 7.4, 1% triton X-100, 1 mM EGTA, 1 mM EDTA and protease (1% v/v; 539134; Calbiochem) and phosphatase (1% v/v; 524625; Calbiochem) inhibitor cocktails. The relative levels of phospho-Ser520-DYRK1A were assayed using western blotting. For this, lysates were diluted into Laemmli sample buffer (Bio-Rad) containing 5% v/v β-mecaptoethanol, heated for 5 min at 95° C., and resolved on Tris-glycine gels or NuPage Bis-Tris gels (Novex; Invitrogen). Biotinylated-molecular weight standards (Cell Signaling Technology) were included in all gels. Proteins were transferred to nitrocellulose membranes (Hybond, ECL; Amersham), which were blocked in Tris-buffered saline/0.1% tween 20 (TBST) containing 5% milk, and probed at 4° C. overnight with anti-phospho-Ser520-DYRK1A antibody (Eurogentec SE6974-75: 0.23 μg/ml in 5% BSA) or anti DYRK1A antibody (Abnova H00001859; 0.5 μg/ml in 5% milk). Peroxidase-conjugated secondary antibodies were diluted into 5% milk and applied to membranes for 1 h at 20° C. Chemiluminescence detection was performed using the ECL plus western blotting detection kit (Amersham) and was recorded on ECL plus hyperfilm (Amersham). Blots were scanned using the Bio-Rad GS-800 calibrated densitometer and quantitative analysis of western blots was performed using TotalLab software (Amersham). The percentage inhibition of phospho-Ser520-DYRK1A as compared to the control tumors was calculated using the ratio between phospho-Ser520-DYRK1A and total DYRK1A signals at each dose. The results showed that the compounds of the invention are powerful inhibitors of tumor DYRK1A Ser520 autophosphorylation.

### EXAMPLE E

### Efficacy Studies in Tumor Xenografts

For anti-tumor efficacy studies, female nude NCr nu/nu mice were injected subcutaneously with U87-MG human glioblastoma cells. When tumors reached a size of approximately 150 mm³, mice were randomized into homogeneous groups of 8 and treated orally with the compounds of the invention at doses of at doses of up to 200 mg/kg once daily for up to 3 weeks. Anti-tumor efficacy was monitored by at least twice-weekly measurement of tumor sizes using calipers, and body weights were recorded in order to document potential general toxicity. Percentage tumor growth inhibition (TGI) on a given day was calculated using the formula: (1-[RTV(treated)/RTV(untreated)])×100, where RTV=relative tumor volume on the given day versus start of treatment. The results showed that the compounds of the invention are powerful inhibitors of tumor growth.

TABLE 1
IC₅₀ of Dvrk1/ClK1 inhibitor

| | IC₅₀ (μM) Dyrk1A | IC₅₀ (μM) Dyrk1A | IC₅₀ (μM) Dyrk1B | IC₅₀ (μM) Clk1 | IC₅₀ (μM) CDK9 | IC₅₀ (μM) P-Ser520- |
|---|---|---|---|---|---|---|
| | TR-FRET assay | ADP assay | TR-FRET assay | TR-FRET assay | TR-FRET assay | Dyrk1A-Cell assay |
| Example 1 | | 0.047 | | | | |
| Example 2 | 0.018 | 0.023 | 0.0222 | | 4.41 | 0.48 |
| Example 3 | | 0.241 | | | | |
| Example 4 | 0.0253 | 0.044 | 0.044 | | 10 | |
| Example 5 | 0.0094 | 0.015 | 0.0005 | | 10 | |
| Example 6 | | 0.07 | | | | |
| Example 7 | | 0.039 | | | | |
| Example 8 | | 0.038 | | | | |
| Example 9 | | 0.06 | | | | |
| Example 10 | | 0.085 | | | | |
| Example 11 | 0.0173 | 0.012 | 0.0132 | | 10 | |
| Example 12 | | 2.041 | | | | |
| Example 13 | | 1.373 | | | | |
| Example 14 | | 0.043 | | | | |
| Example 15 | 0.0355 | 0.032 | 0.0143 | | 10 | |
| Example 16 | 0.0149 | 0.011 | 0.0178 | 0.0328 | 10 | 0.1402 |
| Example 17 | 0.009 | 0.006 | 0.0013 | 0.0166 | 1.8543 | 0.0093 |
| Example 18 | 0.0151 | 0.012 | 0.0003 | 0.024 | 10 | 0.0663 |
| Example 19 | | 0.025 | | | | |
| Example 20 | 0.0197 | 0.013 | | | | |
| Example 21 | 0.0102 | 0.023 | 0.0091 | | 3.7762 | |
| Example 22 | | 0.018 | | | | |
| Example 23 | | 0.015 | | | | |
| Example 24 | | 0.066 | | | | |
| Example 25 | 0.0031 | 0.012 | 0.0079 | 0.0177 | 10 | 0.036 |
| Example 26 | | 0.029 | | | | |
| Example 27 | 0.0444 | 0.04 | 0.0522 | | 10 | |
| Example 28 | | 0.011 | | | | |
| Example 29 | | 0.062 | | | | |
| Example 30 | | 0.827 | | | | |
| Example 31 | | 1.068 | | | | |
| Example 32 | 0.0056 | 0.015 | 0.0012 | | 10 | 0.323 |
| Example 33 | | 0.165 | | | | |
| Example 34 | | 0.278 | | | | |
| Example 35 | 0.0248 | 0.043 | 0.0094 | | 10 | 0.8865 |
| Example 36 | 0.0091 | 0.027 | 0.0062 | | 5.5232 | 0.4857 |
| Example 37 | 0.007 | 0.025 | 0.0005 | | 10 | 0.358 |
| Example 38 | | 0.149 | | | | |
| Example 39 | | 0.084 | | | | |
| Example 40 | | 0.051 | | | | |
| Example 41 | | 0.158 | | | | |
| Example 42 | | 0.233 | | | | |
| Example 43 | | 0.278 | | | | |
| Example 44 | | 0.249 | | | | |
| Example 45 | 0.2005 | 0.496 | | | 30 | 0.6864 |
| Example 46 | | 0.369 | | | | |
| Example 47 | | 0.372 | | | | |
| Example 48 | 0.043 | 0.044 | | | 10 | 0.208 |
| Example 49 | | 0.127 | | | | |
| Example 50 | | 0.045 | | | | |
| Example 51 | 0.0029 | 0.013 | | | 10 | 0.126 |
| Example 52 | 0.0043 | 0.007 | 0.0027 | 0.0167 | 10 | 0.0232 |
| Example 53 | 0.0233 | 0.021 | | | 10 | 0.2375 |
| Example 54 | 0.0129 | 0.032 | | | 10 | 0.5105 |
| Example 55 | 0.0102 | 0.009 | 0.0043 | 0.0157 | 1.3025 | 0.0058 |
| Example 56 | 0.0114 | 0.012 | | | 2.5354 | 0.0117 |
| Example 57 | 0.0026 | 0.015 | 0.0098 | 0.0233 | 8.0604 | 0.0497 |
| Example 58 | 0.0215 | 0.01 | 0.0175 | 0.0245 | 10 | 0.0337 |
| Example 59 | 0.0102 | 0.042 | 0.0191 | | 10 | 0.2587 |
| Example 60 | 0.003 | 0.011 | | | 10 | 0.0206 |
| Example 61 | 0.0062 | 0.01 | 0.0029 | 0.0129 | 10 | 0.0115 |
| Example 62 | 0.0186 | 0.008 | 0.0002 | 0.0162 | 10 | 0.021 |
| Example 63 | 0.0107 | 0.014 | | | 10 | 0.0408 |
| Example 64 | 0.0059 | 0.015 | 0.0093 | | 10 | 0.2335 |
| Example 65 | 0.0709 | 0.069 | | | 30 | 0.8984 |
| Example 66 | 0.0107 | 0.045 | | | 10 | 0.3 |
| Example 67 | | 0.094 | | | | |
| Example 68 | | 0.059 | | | | |
| Example 69 | 0.0016 | 0.006 | 0.0011 | | 0.6478 | 0.0036 |
| Example 70 | 0.0025 | 0.009 | 0.0015 | 0.0152 | 1.5031 | 0.027 |
| Example 71 | 0.0051 | 0.008 | 0.0074 | 0.0237 | 10 | 0.031 |
| Example 72 | 0.021 | 0.013 | | | 10 | 0.3 |
| Example 73 | 0.0059 | 0.038 | | | 10 | 0.3 |
| Example 74 | 0.0012 | 0.014 | | 0.0184 | 10 | 0.1115 |
| Example 75 | 0.0143 | 0.037 | | | 10 | 0.3 |
| Example 76 | 0.0063 | 0.01 | 0.0005 | | 10 | 0.0672 |
| Example 77 | | 0.057 | | | | |
| Example 78 | 0.0013 | 0.01 | 0.0145 | 0.0293 | 10 | 0.0721 |
| Example 79 | 0.0021 | 0.008 | 0.008 | | 10 | 0.105 |
| Example 80 | 0.0059 | 0.004 | 0.0106 | | 10 | 0.0156 |
| Example 81 | 0.0085 | 0.014 | 0.0141 | | 10 | 0.1659 |
| Example 82 | 0.001 | 0.045 | 0.0199 | | 10 | |
| Example 83 | 0.0006 | 0.081 | 0.0404 | | 10 | |
| Example 84 | | 0.006 | | | | 0.0097 |
| Example 86 | | 0.121 | | | | |
| Example 87 | | 1.939 | | | | |
| Example 88 | | 2.091 | | | | |
| Example 89 | 0.0492 | 0.077 | | | 30 | |
| Example 90 | | 10 | | | | |
| Example 91 | | 0.038 | | | | |
| Example 92 | | 0.087 | | | | |
| Example 93 | | 0.176 | | | | |
| Example 94 | 0.0077 | 0.019 | 0.0112 | 0.0378 | 3 | 0.1549 |
| Example 95 | 0.0979 | 0.066 | | | 30 | 0.5344 |
| Example 96 | 0.0023 | 0.009 | 0.0315 | 0.0151 | 3 | 0.0119 |
| Example 97 | | 0.063 | | | | |
| Example 98 | | 0.022 | | 0.0241 | | 0.1923 |
| Example 99 | 0.0086 | 0.029 | 0.0293 | 0.0549 | 3 | 0.1921 |
| Example 100 | | 0.161 | | | | |
| Example 101 | | 0.034 | | | | 0.3 |
| Example 102 | | 0.293 | | | | |
| Example 103 | | 0.694 | | | | |
| Example 104 | 0.0081 | 0.015 | 0.0167 | 0.0225 | 3 | 0.1055 |
| Example 105 | | 0.121 | | | | |
| Example 106 | | 0.018 | | 0.0171 | | 0.1769 |
| Example 107 | | 0.666 | | | | |
| Example 108 | 0.0027 | 0.009 | 0.0092 | 0.0283 | 3 | 0.0491 |
| Example 109 | | 0.524 | | | | |
| Example 110 | | 0.048 | | | | |
| Example 111 | | 0.013 | | | | |
| Example 112 | | 0.234 | | | | |
| Example 113 | | 0.114 | | | | |
| Example 114 | | 0.009 | | 0.0162 | | 0.006 |
| Example 115 | 0.0031 | 0.005 | 0.0094 | 0.0172 | 3 | 0.0185 |
| Example 116 | | 0.005 | | 0.0136 | | 0.0009 |
| Example 117 | 0.0059 | 0.01 | 0.0093 | 0.0195 | | 0.0377 |
| Example 118 | | 0.011 | | | | |
| Example 119 | 0.0066 | 0.02 | 0.0192 | 0.0828 | 3 | 0.2317 |
| Example 120 | | 0.115 | | | | |
| Example 121 | | 0.066 | | | | |
| Example 122 | | 0.05 | | | | |
| Example 123 | | 0.071 | | 0.0615 | | 0.3 |
| Example 124 | | 0.296 | | | | |
| Example 125 | 0.053 | 0.073 | | | 3.72 | |
| Example 126 | | 0.418 | | | | |
| Example 127 | | 0.011 | | | | 0.0169 |
| Example 128 | | 0.009 | | | | 0.0093 |
| Example 129 | | 0.072 | | | | |
| Example 130 | | 0.26 | | | | |
| Example 131 | | 0.6 | | | | |
| Example 132 | 0.0338 | 0.122 | | | 30 | |
| Example 133 | | 0.269 | | | | |
| Example 134 | | 0.848 | | | | |
| Example 135 | | 0.091 | | | | |
| Example 136 | | 0.169 | | | | |
| Example 137 | | 0.336 | | | | |
| Example 138 | | 0.407 | | | | |
| Example 139 | | 0.883 | | | | |
| Example 140 | | 1.223 | | | | |
| Example 141 | | 0.417 | | | | |
| Example 142 | | 0.512 | | | | |
| Example 143 | | 1.057 | | | | |
| Example 144 | | 0.545 | | | | |
| Example 145 | | 0.042 | | | | 0.4706 |
| Example 146 | | 0.172 | | | | |
| Example 147 | | 0.17 | | | | |
| Example 148 | 0.0042 | 0.007 | 0.0144 | 0.0303 | 10 | 0.0335 |
| Example 149 | | 0.734 | | | | |
| Example 150 | 0.0034 | 0.74 | | | 1.1651 | |
| Example 151 | | 0.028 | | | | |
| Example 152 | | 0.012 | | | | 0.0101 |
| Example 153 | | 0.011 | | | | 0.0146 |
| Example 154 | | 0.013 | | | | 0.053 |
| Example 155 | | 0.024 | | | | 0.3 |
| Example 156 | | 0.029 | | | | |
| Example 157 | | 0.26 | | | | |
| Example 158 | 0.0655 | 0.15 | | | 30 | |
| Example 159 | | 0.012 | | | | 0.0187 |
| Example 160 | | 0.184 | | | | |
| Example 161 | 0.0091 | 0.028 | 0.0252 | 0.1222 | 3 | 0.1501 |
| Example 162 | | 0.014 | | | | |
| Example 163 | | 0.026 | | 0.1041 | | 0.1974 |
| Example 164 | | 0.015 | | | | 0.0883 |
| Example 165 | | 0.301 | | | | |
| Example 166 | | 0.025 | | 0.2476 | | 0.1179 |
| Example 167 | | 0.015 | | 0.042 | | 0.0444 |
| Example 168 | | 0.01 | | | | |
| Example 169 | | 0.216 | | | | |
| Example 170 | | 1.824 | | | | 0.3 |
| Example 171 | | 0.033 | | | | |
| Example 172 | | 0.037 | | | | |
| Example 173 | 0.0045 | 0.013 | 0.0051 | 0.0334 | 3 | 0.0497 |
| Example 174 | | 0.07 | | | | |
| Example 175 | | 0.146 | | | | |
| Example 176 | | 0.196 | | | | |
| Example 177 | | 0.532 | | | | |
| Example 178 | 0.0052 | 0.013 | 0.0141 | 0.1795 | 3 | 0.0782 |
| Example 179 | 0.0031 | 0.014 | 0.0115 | 0.0425 | 10 | 0.0365 |
| Example 180 | | 0.079 | | | | |
| Example 181 | | 0.019 | | | | 0.15 |
| Example 182 | | 0.013 | | | | 0.0142 |
| Example 183 | | 0.006 | | | | 0.029 |
| Example 184 | | 0.012 | | | | 0.0319 |
| Example 185 | 0.0048 | 0.011 | 0.0158 | 0.0631 | 10 | 0.012 |
| Example 186 | 0.0053 | 0.017 | 0.0211 | 0.0927 | 10 | 0.0855 |
| Example 187 | 0.003 | 0.013 | 0.0081 | 0.0649 | 11.639 | 0.0342 |
| Example 188 | | 0.07 | | | | |
| Example 189 | | 0.062 | | | | 0.3 |
| Example 190 | | 0.419 | | | | |
| Example 191 | | 0.006 | | | | 0.0443 |
| Example 192 | | 0.008 | | | | 0.048 |
| Example 193 | | 0.116 | | | | |
| Example 194 | | 0.007 | | | | 0.017 |
| Example 195 | | 0.008 | | | | 0.0071 |
| Example 196 | | 0.023 | | | | 0.1715 |
| Example 197 | | 0.009 | | | | |
| Example 198 | | 0.017 | | | | 0.1193 |
| Example 199 | | 0.148 | | | | |
| Example 200 | | 0.027 | | | | |
| Example 201 | | 0.012 | | | | |
| Example 202 | | 0.144 | | | | |
| Example 203 | | 0.155 | | | | |
| Example 204 | | 0.089 | | | | |
| Example 205 | | 0.055 | | | | |
| Example 206 | 0.0071 | 0.008 | 0.0133 | 0.0238 | 10 | 0.0015 |
| Example 207 | 0.0049 | 0.01 | 0.0181 | 0.0545 | 7.8293 | 0.0694 |
| Example 208 | | 0.01 | | | | 0.0041 |
| Example 209 | | 0.032 | | | | 0.1571 |
| Example 210 | | 0.05 | | | | 0.3 |
| Example 211 | | 0.027 | | | | |
| Example 212 | | 0.009 | | | | |
| Example 213 | 0.0026 | 0.008 | 0.0092 | 0.0172 | 3 | 0.057 |
| Example 214 | | 0.242 | | | | |
| Example 215 | | 0.019 | | | | 0.1032 |
| Example 216 | 0.0145 | 0.021 | 0.0219 | 0.1212 | 10 | 0.0807 |
| Example 217 | 0.0027 | 0.01 | 0.0077 | 0.0261 | 10 | 0.0385 |
| Example 218 | | 0.015 | | | | 0.0289 |
| Example 219 | | 0.019 | | | | 0.0325 |
| Example 220 | | 0.034 | | | | 0.1933 |
| Example 221 | | 0.016 | | | | 0.1323 |
| Example 222 | | 0.008 | | | | 0.0442 |
| Example 223 | | 0.043 | | | | 0.3 |
| Example 224 | | 0.03 | | | | 0.2249 |
| Example 225 | | 0.037 | | | | |

### EXAMPLE F

### Pharmaceutical Composition: Tablets

| | |
|---|---|
| 1000 tablets containing a dose of 5 mg of a | 5 g |
| compound selected from Examples 1 to 225 | |
| Wheat starch | 20 g |
| Maize starch | 20 g |
| Lactose | 30 g |
| Magnesium stearate | 2 g |
| Silica | 1 g |
| Hydroxypropylcellulose | 2 g |

1-37. (canceled)

38. A compound of formula (I): 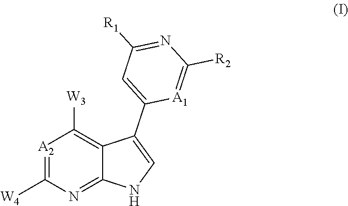
wherein:
R₁ and R₂, each independently of the other, represent a hydrogen atom, a halogen atom, —NR₅R₅′ or a linear or branched (C₁-C₆)alkyl group,
W₃ represents a linear or branched (C₁-C₆)alkoxy, —O—(C₁-C₆)alkylene-Cy₁, —O—(C₀-C₆)-Cy₁-Cy₂, —NRₐR_{b}, —NRₐ—(C₀-C₆)alkylene-Cy₁, —NRₐ-(C₀-C₆)alkylene-Cy₁-Cy₂, —NRₐ—(C₀-C₆)alkylene-Cy₁-O—(C₁-C₆)alkylene-Cy₂, -Cy₁, -Cy₁-(C₀-C₆)alkylene-Cy₂, -Cy₁-O—(C₀-C₆)alkylene-Cy₂, —(C₁-C₆)alkylene-Cy₁, —(C₂-C₆)alkenylene-Cy₁, —(C₂-C₆)alkynylene-Cy₁, —(C₁-C₆)alkylene-O-Cy₁, it being understood that the alkylene moieties defined hereinbefore may be linear or branched,
W₄ represents a cyano group, a cycloalkyl group, a linear or branched (C₁-C₆)alkyl group, a linear or branched (C₂-C₆)alkenyl group, a linear or branched (C₂-C₆)alkynyl group optionally substituted by a cycloalkyl group,
R₅ and R₅′ each independently of the others, represent a hydrogen atom or a linear or branched (C₁-C₆)alkyl group,
Rₐ and R_{b}, each independently of the other, represent a hydrogen atom or a linear or branched (C₁-C₆)alkyl group,
A₁ and A₂, each independently of the other, represent CH or a nitrogen atom,
Cy₁, Cy₂and Cy₃, independently of one another, represent a cycloalkyl group, a heterocycloalkyl group, an aryl or an heteroaryl group,
wherein:
“aryl” means a phenyl, naphthyl, biphenyl or indenyl group,
“heteroaryl” means any mono- or bi-cyclic group composed of from 5 to 10 ring members, having at least one aromatic moiety and containing from 1 to 4 hetero atoms selected from oxygen, sulphur and nitrogen.
“cycloalkyl” means any mono- or bi-cyclic, non-aromatic, carbocyclic group containing from 3 to 11 ring members, which may include fused, bridged or spiro ring systems,
“heterocycloalkyl” means any mono- or bi-cyclic, non-aromatic, condensed or spiro group composed of from 3 to 10 ring members and containing from 1 to 3 hetero atoms or groups selected from oxygen, sulphur, SO, SO₂ and nitrogen, which may include fused, bridged or spiro ring systems,
“—(C₀-C₆)alkylene-” refers either to a covalent bond (—C₀alkylene-) or to an alkylene group containing 1, 2, 3, 4, 5 or 6 carbon atoms,
wherein the aryl, heteroaryl, cycloalkyl and heterocycloalkyl groups so defined and the alkyl, alkenyl, alkynyl, alkylene, alkenylene, alkynylene to be substituted by from 1 to 4 groups selected from linear or branched (C₁-C₆)alkyl, linear or branched (C₂-C₆)alkenyl group, linear or branched (C₂-C₆)alkynyl group, linear or branched (C₁-C₆)alkoxy optionally substituted by —NR_{c}R_{d} or by from 1 to 3 halogen atoms, linear or branched (C₁-C₆)alkyl-S—, hydroxy, oxo (or N-oxide where appropriate), nitro, cyano, —C(O)—OR_{c}, —C(O)—R_{c}, —O—C(O)—R_{d}, —C(O)—NR_{c}R_{d}, —NR_{c}—C(O)—R_{d}, —NR_{c}R_{d}, linear or branched (C₁-C₆)polyhaloalkyl, or halogen, it being understood that R_{c} and R_{d} independently of one another represent a hydrogen atom or a linear or branched (C₁-C₆)alkyl group,
its enantiomers and diastereoisomers, and addition salts thereof with a pharmaceutically acceptable acid or base.

39. The compound according to claim 38, wherein R₁ represents a hydrogen and R₂ represents a —NH₂ group.

40. The compound according to claim 38, wherein A₁ represents a CH group.

41. The compound according to claim 38, wherein A₁ represents a nitrogen atom.

42. The compound according to claim 38, wherein A₂ represents a nitrogen atom.

43. The compound according to claim 38, wherein A₂ represents a CH group.

44. The compound according to claim 43, wherein A₂ represents a CH group and A₁ represents a CH group.

45. The compound according to claim 38, wherein W₃ represents a linear or branched (C₁-C₆)alkoxy, —O—(C₀-C₆)alkylene-Cy₁, —O—(C₀-C₆)alkylene-Cy₁-Cy₂, —NRₐ—(C₁-C₆)alkylene-Cy₁Cy₂, —NRₐ—(C₀-C₆)alkylene-Cy₁-O—(C₁-C₆)alkylene-Cy₂, —Cy₁-O—(C₀-C₆)alkylene-Cy₂, —(C₁-C₆)alkenylene-Cy₁, —(C₂-C₆)alkynylene-Cy₁, —(C₂-C₆)alkynylene-Cy₁, —(C₁-C₆)alkylene-O-Cy₁, wherein the alkylene moieties defined hereinbefore may be linear or branched.

46. The compound according to claim 38, wherein W₃ represents a Cy₁ group selected from: 1,3-benzodioxolyl, 1H-indolyl, phenyl, pyridinyl, 2,3-dihydro-1,4-benzodioxinyl, 1-benzothiophenyl, 1-benzofuranyl, 3,4-dihydronaphthalenyl, 1,2,3,4-tetrahydronaphthalenyl, 3,4-dihydro-2H-1,4-benzoxazinyl, wherein the preceding groups are optionally substituted by from 1 to 4 groups selected from linear or branched (C₁-C₆)alkyl, linear or branched (C₂-C₆)alkenyl, linear or branched (C₂-C₆)alkynyl, linear or branched (C₁-C₆)alkoxy optionally substituted by —NR_{c}R_{d} or by from 1 to 3 halogen atoms, linear or branched (C₁-C₆)alkyl-S—, hydroxy, oxo (or N-oxide where appropriate), nitro, cyano, —C(O)—OR_{c}, —C(O)—R_{c}, —O—C(O)—R_{d}, —C(O)—NR_{c}R_{d}, —NR_{c}—C(O)—R_{d}, —NR_{c}R_{d}, linear or branched (C₁-C₆)polyhaloalkyl, or halogen, wherein R_{c} and R_{d} independently of one another represent a hydrogen atom or a linear or branched (C₁-C₆)alkyl group.

47. The compound according to claim 38, wherein W₃ represents:
(i) a —NRₐ-Cy₁ group, wherein Cy₁ represents a group selected from: phenyl, 2,3-dihydro-1H-indene and 1,2,3,4-tetrahydronaphthalene, wherein the preceding groups are optionally substituted by from 1 to 4 groups selected from linear or branched (C₁-C₆)alkyl, linear or branched (C₂-C₆)alkenyl, linear or branched (C₂-C₆)alkynyl, linear or branched (C₁-C₆)alkoxy optionally substituted by —NR_{c}R_{d} or by from 1 to 3 halogen atoms, linear or branched (C₁-C₆)alkyl-S—, hydroxy, oxo (or N-oxide where appropriate), nitro, cyano, —C(O)—OR_{c}, —C(O)—R_{c}, —O—C(O)—R_{d}, —C(O)—NR_{c}R_{d}, —NR_{c}—C(O)—R_{d}, —NR_{c}R_{d}, linear or branched (C₁-C₆)polyhaloalkyl, or halogen, wherein R_{c} and R_{d} independently of one another represent a hydrogen atom or a linear or branched (C₁-C₆)alkyl group; or
(ii) a —NRₐ—(C₁-C₆)alkylene-Cy₁ group, wherein Cy₁ represents a group selected from: phenyl, pyridinyl, furanyl, thiophenyl, 1H-pyrazolyl, 1,3-thiazolyl, 1,2-oxazolyl, cyclohexyl, cyclopropyl and 1H-indolyl, wherein the preceding groups are optionally substituted by from 1 to 4 groups selected from linear or branched (C₁-C₆)alkyl, linear or branched (C₂-C₆)alkenyl, linear or branched (C2-C6)alkynyl, linear or branched (C₁-C₆)alkoxy optionally substituted by —NR_{c}R_{d} or by from 1 to 3 halogen atoms, linear or branched (C₁-C₆)alkyl-S—, hydroxy, oxo (or N-oxide where appropriate), nitro, cyano, —C(O)—OR_{c}, —C(O)—R_{c}, —O—C(O)—R_{d}, —C(O)—NR_{c}R_{d}, —NR_{c}—C(O)—R_{d}, —NR_{c}R_{d}, linear or branched (C₁-C₆)polyhaloalkyl, or halogen, wherein R_{c} and R_{d} independently of one another represent a hydrogen atom or a linear or branched (C₁-C₆)alkyl group.

48. The compound according to claim 38, wherein W₃ represents a -phenylene-(C₀-C₆)alkylene-Cy₂.

49. The compound according to claim 38, wherein W₃ represents —O—(C₁-C₆)alkylene-Cy₁ or —NRₐ—(C₁-C₆)alkylene-Cy₁, wherein Cy₁ is a phenyl or a pyridinyl group, these latter groups being optionally substituted by one or two groups selected from methoxy, methyl and halogen.

50. The compound according to claim 38, wherein W₄ is methyl, propan-2-yl, prop-1-en-2-yl, ethenyl, cyano, ethynyl, cyclopropyl or cyclopropylethynyl.

51. The compound according to claim 50, wherein W₄ is methyl.

52. The compound according to claim 38, which is selected from the group consisting of:
5-(2-aminopyridin-4-yl)-N-(2-methoxybenzyl)-2-methyl-7H-pyrrolo[2,3-d]pyrimidin-4-amine,
4-[2-methyl-4-(thiophen-3-ylmethoxy)-7H-pyrrolo[2,3-d]pyrimidin-5-yl]pyridin-2-amine,
5-(2-aminopyridin-4-yl)-N-(2,6-dichlorobenzyl)-2-methyl-7H-pyrrolo[2,3-d]pyrimidin-4-amine,
5-(2-aminopyridin-4-yl)-N-(2,6-difluorobenzyl)-2-methyl-7H-pyrrolo[2,3-d]pyrimidin-4-amine,
5-(2-aminopyridin-4-yl)-2-methyl-N-(2-methylbenzyl)-7H-pyrrolo[2,3-d]pyrimidin-4-amine,
5-(2-aminopyridin-4-yl)-N-(2-chloro-6-fluorobenzyl)-2-methyl-7H-pyrrolo[2,3-d]pyrimidin-4-amine,
5-(2-aminopyridin-4-yl)-2-methyl-N-[(3-methylpyridin-2-yl)methyl]-7H-pyrrolo[2,3-d]pyrimidin-4-amine,
5-(2-aminopyridin-4-yl)-N-[(3-fluoropyridin-2-yl)methyl]-2-methyl-7H-pyrrolo[2,3-d]pyrimidin-4-amine,
5-(2-aminopyridin-4-yl)-N-(2,6-difluorobenzyl)-2-methyl-7H-pyrrolo[2,3-d]pyrimidin-4-amine, and
its enantiomers and diastereoisomers, and addition salts thereof with a pharmaceutically acceptable acid or base.

53. The compound according to claim 38, which is 5-(2-aminopyridin-4-yl)-N-(2,6-dichlorobenzyl)-2-methyl-7H-pyrrolo[2,3-d]pyrimidin-4-amine.

54. The compound according to claim 38, which is 5-(2-aminopyridin-4-yl)-N-(2,6-difluorobenzyl)-2-methyl-7H-pyrrolo-[2,3-d]pyrimidin-4-amine.

55. The compound according to claim 38, which is 5-(2-aminopyridin-4-yl)-N-(2-chloro-6-fluorobenzyl)-2-methyl-7H-pyrrolo-[2,3-d]pyrimidin-4-amine.

56. The compound according to claim 38, which is 5-(2-aminopyridin-4-yl)-2-methyl-N-[(3-methylpyridin-2-yl)methyl]-7H-pyrrolo-[2,3-d]pyrimidin-4-amine.

57. The compound according to claim 38, which is 5-(2-aminopyridin-4-yl)-N-[(3-fluoropyridin-2-yl)methyl]-2-methyl-7H-pyrrolo-[2,3-d]pyrimidin-4-amine.

58. The compound according to claim 38, which is 5-(2-aminopyrimidin-4-yl)-N-(2,6-difluorobenzyl)-2-methyl-7H-pyrrolo-[2,3-d]pyrimidin-4-amine.

59. A pharmaceutical composition comprising the compound according to claim 38, or an addition salt thereof with a pharmaceutically acceptable acid or base, in combination with one or more pharmaceutically acceptable excipients.

60. A method of treating a condition selected from cancer, neurodegenerative disorders and metabolic disorders in a subject in need thereof, comprising administration of an effective amount of the compound according to claim 38, alone or in combination with one or more pharmaceutically acceptable excipients.

61. The method according to claim 60, wherein the cancer is selected from acute megakaryoblastic leukaemia (AMKL), acute lymphoblastic leukaemia (ALL), ovarian cancer, pancreatic cancer, gastrointestinal stromal tumours (GIST), osteosarcoma (OS), colorectal carcinoma (CRC), neuroblastoma and glioblastoma.

62. The method according to claim 60, wherein the neurodegenerative disorders are selected from Alzheimer's, Parkinson's and Huntington's diseases, Down's syndrome, mental retardation and motor defects.

63. A combination of the compound according to claim 38 with an anticancer agent selected from genotoxic agents, mitotic poisons, anti-metabolites, proteasome inhibitors, kinase inhibitors, signaling pathway inhibitors, phosphatase inhibitors, apoptosis inducers and antibodies.

64. A pharmaceutical composition comprising the combination according to claim 63 in combination with one or more pharmaceutically acceptable excipients.

65. A method of treating cancer in a subject in need thereof comprising administration of the combination according to claim 63, alone or in combination with one or more pharmaceutically acceptable excipients.

66. A method of treating cancer necessitating radiotherapy in a subject in need thereof, comprising administration of the compound according to claim 38, alone or in combination with one or more pharmaceutically acceptable excipients.
